(19) United States
(12) Patent Application Publication
(10) Pub. No.: US 2024/0366717 A1
(43) Pub. Date: Nov. 7, 2024

(54) ANTIMICROBIAL MATRIX FORMED FROM PEPTIDE HYDROGELS

(71) Applicant: GEL4MED, INC., Brighton, MA (US)

(72) Inventors: Kushee-Nidhi Kush Kumar, Medford, MA (US); Ravi Mekala, Boston, MA (US); Manav Mehta, Newton, MA (US)

(21) Appl. No.: 18/040,388

(22) PCT Filed: Aug. 9, 2021
(86) PCT No.: PCT/US2021/045270

§ 371 Date: Feb. 2, 2023

Related U.S. Application Data
(60) Provisional application No. 63/063,764, filed on Aug. 10, 2020.

(51) Int. Cl. A61K 38/17; A01N 63/50; A01P 1/00; A61K 9/06; A61L 2/00; A61L 2/18; A61L 101/46; A61P 31/02

(52) U.S. Cl. CPC A61K 38/1729; A01N 63/50; A01P 1/00; A61K 9/06; A61L 2/0088; A61L 2/18; A61P 31/02; A61L 2101/46; A61L 2202/22; A61L 2202/24

(57) ABSTRACT

Methods of treating a microbial contamination are disclosed. Methods of eliminating or inhibiting proliferation of a target microorganism at a target site are also disclosed. The methods include administering a thermally stable preparation comprising a purified amphiphilic peptide in an aqueous biocompatible solution and administering a buffer to a target site. The peptide has a folding group having a plurality of charged amino acid residues and hydrophobic amino acid residues arranged in a substantially alternating pattern and a turn sequence. The peptide is configured to self-assemble into a hydrogel.

### CROSS-REFERENCE TO RELATED APPLICATIONS

This application claims priority under 35 U.S.C. § 119(e) to U.S. Provisional Patent Application No. 63/063,764 titled “Antimicrobial Matrix Formed from Peptide Hydrogels” filed Aug. 10, 2020, the entire disclosure of which is herein incorporated by reference in its entirety for all purposes.

### STATEMENT REGARDING FEDERALLY SPONSORED RESEARCH

This invention was made with government support under Small Business Innovation Research (SBIR) Grant Nos. R43GM117858-01 and R43GM122226, awarded by the National Institutes of Health (NIH). The government has certain rights in the invention.

### SEQUENCE LISTING

The instant application contains a Sequence Listing which has been submitted electronically in ASCII format and is hereby incorporated by reference in its entirety. Said ASCII copy, created on Aug. 6, 2021, is named G2093-7003WOFSR_SL.txt and is 9,679 bytes in size.

### FIELD OF TECHNOLOGY

Aspects and embodiments disclosed herein are directed toward preparations and methods for treatment of antimicrobial infections. In particular, aspects and embodiments disclosed herein are directed toward preparations and methods for treatment of surgical site infections.

### BACKGROUND

Wound healing is the process by which epidermal layers repair themselves after injury. The wound healing process is complex and susceptible to complications from the formation of non-healing chronic wounds. Factors that contribute to chronic wounds include diabetes, venous or arterial disease, infection, and metabolic deficiencies.

Surgical site infection (SSI) is one of the most common surgical complications. The need for preventing and treating surgical site infections is critical. SSI is the second most common type of health care-associated infection occurring in 2% to 5% of patients undergoing surgery in the United States. As many as 15 million procedures are annually performed in the United States. Thus, as many as 300,000 to 500,000 SSIs occur as complications each year. SSI has the ability to increase mortality risk 2 to 11-fold, with 77% of deaths in patients with SSI directly attributed to the SSI. These infections have resulted in 3.7 In the future, as the population ages with increasing risk factors, the incidence of SSI is expected to rise. Reducing SSIs is a national priority across developed and developing countries alike, indicating a compelling need for modalities associated with pre and post-surgical care.

Both surgical wounds in civilian population and battlefield extremity wounds are easily contaminated resulting in a disproportionally high mortality rate. Infected patients end up suffering from severe consequences that often lead to amputation and inability to return to duty for soldiers. Recovery becomes more complicated with the emergence of multi-drug resistant organisms (MDROs). MDROs constitute a global leading health threat with steadily increasing morbidity and mortality rates. Unfortunately, limited prophylactic and/or therapeutic options are available for combat casualty care at the point of injury, the “Golden Hour”, where the opportunity to prevent an infection is the highest.

Antibiotics are systemic and need appropriate dosing, timing, and close monitoring for effectiveness. Moreover, constant antibiotic use reinforces antibiotic resistance, generating a vicious cycle with no obvious benefit and enhancing a long-term perplexing reality. Recent conflicts are an emphatic example for need of rapid and easy to apply prophylactic antimicrobial regiments to support deployed forces. The incidence of wounded soldiers being infected abroad with unconventional pathogens, such as multidrug resistant Acinetobacter spp., is evidence of the need for a strong commitment to a different approach.

Prophylactic therapy and treatment are of utmost importance for in-point prevention, therapy, and treatment of wounds. Antimicrobial prophylactic countermeasures found in a self-aid kit or medical bag should conform to the following criteria: (i) efficacy, (ii) ability to reduce potential for developing drug resistance, (iii) thermo-stability, (iv) portability, (v) ease of administration, (vi) flexibility to conform to different types and shapes of wounds, (vii) hemostatic ability, and (viii) biocompatibility. Improved wound-treatment prophylactic therapies are needed.

Studies have found bacterial biofilms in 60-70% of chronic wounds and 6% of acute wounds. Biofilms stimulate chronic inflammation, including increased proinflammatory cytokines, proteases, reactive oxygen species, and the degradation of proteins needed for healing. The effects include delayed wound closure, amputation, frequent surgical interventions, and extended use of antibiotic and antifungal treatments. According to a 2017 report on non-healing biofilm-associated chronic wounds by an expert panel—(i) biofilm treatment with antibiotic or antiseptic fails frequently, (ii) biofilms impair wound healing and are prone to cycles of recurrent infection/exacerbation, and (iii) biofilm prevention and treatment should be considered early for wounds at high risk for infections and dehiscence. Although wound debridement is critical for reducing biofilm, alone or in combination with systemic antibiotics, it does not remove all biofilm. New treatments are needed to inhibit biofilm formation and prevent reformation after wound debridement.

### SUMMARY

In accordance with one aspect, there is provided a method of treating a microbial contamination of a subject in need thereof. The method may comprise administering to a target site of the subject a thermally stable preparation comprising a purified amphiphilic peptide in an aqueous biocompatible solution in an amount effective to promote deactivation of a target microorganism associated with the microbial contamination. The peptide may comprise a folding group having a plurality of charged amino acid residues and hydrophobic amino acid residues arranged in a substantially alternating pattern and a turn sequence. The peptide may be configured to self-assemble into a hydrogel. The method may comprise administering to the target site a buffer comprising an effective amount of an ionic salt and a biological buffering agent to form the hydrogel.

In accordance with another aspect, there is provided a method of eliminating or inhibiting proliferation of a target microorganism at a target site. The method may comprise administering to the target site an effective amount of a thermally stable preparation comprising a purified amphiphilic peptide in an aqueous biocompatible solution. The peptide may comprise a folding group having a plurality of charged amino acid residues and hydrophobic amino acid residues arranged in a substantially alternating pattern and a turn sequence. The peptide may be configured to self-assemble into a hydrogel. The method may comprise administering to the target site a buffer comprising an effective amount of an ionic salt and a biological buffering agent to form the hydrogel.

In some embodiments, the target site may by a local site of the microbial contamination.

The peptide may comprise an effective amount of counterions.

The peptide may comprise an effective amount of acetate, citrate, and/or chloride counterions.

The peptide may be substantially free of chloride counterions.

The buffer may comprise between about 10 mM and 150 mM sodium chloride and between about 10 mM and 100 mM Bis-tris propane (BTP).

In some embodiments, the amount is sufficient to sterilize at least 90% of the target microorganism at the target site, for example, at least 95%, at least 98%, at least 99%, at least 99.99%, or at least 99.999%.

In some embodiments, the target microorganism is a pathogenic microorganism.

The target microorganism may be a species of a genus selected from Bacillus, Bartonella, Bordetella, Borrelia, Brucella, Campylobacter, Chlamydia, Chlamydophila, Clostridioides, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yersinia.

The target site may be a tissue selected from mesenchymal tissue, connective tissue, muscle tissue, nervous tissue, embryonic tissue, dermal tissue, bone tissue, dental tissue, corneal tissue, cutaneous tissue, integumental tissue, soft tissue, and hard tissue.

The target site may be a biological fluid selected from tears, mucus, urine, menses, blood, wound exudates, and mixtures thereof.

The target site may be a surface selected from a household surface, an industrial surface, a food industry surface, and a healthcare surface.

The target surface may be a medical tool surface, a medical implant surface, or a medical device surface.

The method may comprise administering the preparation to the target site of a subject topically, enterally, or parenterally.

The method may comprise administering the preparation by spray, dropper, film, squeeze tube, or syringe.

The method may comprise administering the preparation in combination with a surgical procedure.

The method may comprise administering a first dosage of the preparation.

The method may comprise administering at least one booster dosage of the preparation.

The hydrophobic amino acid residues may be independently selected from glycine, alanine, valine, leucine, isoleucine, proline, phenylalanine, methionine, threonine, tryptophan, and combinations thereof.

The charged amino acid residues may be independently selected from arginine, lysine, histidine, and combinations thereof.

In some embodiments, the folding group has a sequence comprising Y[XY]_{N}[T][YX]_{M}Y, where X is 1-3 charged amino acids, Y is 1-3 hydrophobic amino acids, T is 2-8 turn sequence amino acids, and N and M are each independently between 2 and 10.

In some embodiments, the turn sequence amino acids may be independently selected from a D-proline, an L-proline, aspartic acid, threonine, asparagine, and combinations thereof.

The peptide may be configured to self-assemble into a substantially biocompatible hydrogel.

The peptide may be configured to self-assemble into a cell friendly hydrogel.

The peptide may be configured to self-assemble into a substantially biodegradable, non-inflammatory, and/or non-toxic hydrogel.

The peptide may be configured to self-assemble into a hydrogel having substantially low hemolytic activity.

The peptide may be configured to self-assemble into a hydrogel having substantially low immunogenic activity.

The method may further comprise administering at least one combination treatment selected from: an antibacterial composition, an antifungal composition, an antiviral composition, an anti-tumor composition, an anti-inflammatory composition, a cell culture media, a cell culture serum, an anti-odor composition, a hemostatic composition, and an analgesic or pain-relief composition.

The combination treatment may be administered prior to the preparation.

The combination treatment may be administered after the preparation.

The combination treatment may be administered concurrently with the preparation.

The method may comprise combining the preparation and the buffer prior to administration.

The method may comprise combining the preparation and the buffer less than about 1 minute, less than about 2 minutes, less than about 5 minutes, or less than about 10 minutes prior to administration.

The method may comprise combining the preparation and the buffer at a point of use.

The peptide may be at least 80% purified, for example, at least 85%, at least 90%, at least 92%, at least 95%, at least 98%, at least 99%, or at least 99.9%.

The purified peptide may have less than 10% residual organic solvent by weight, for example, less than 8%, less than 5%, less than 2%, less than 1%, or less than 0.1%.

The organic solvent may comprise at least one of trifluoroacetic acid (TFA), acetonitrile, isopropanol, N,N-Dimethylformamide, triethylamine, Ethyl Ether, and acetic acid.

The preparation may have a residual Trifluoroacetic acid (TFA) concentration of less than about 1% w/v, a residual acetonitrile concentration of less than about 410 ppm, a residual N,N-Dimethylformamide concentration of less than about 880 ppm, a residual triethylamine concentration of less than about 5000 ppm, a residual Ethyl Ether concentration of less than about 1000 ppm, a residual isopropanol concentration of less than about 100 ppm, and/or a residual acetic acid concentration of less than 0.1% w/v.

The peptide may include a functional group.

The functional group may have between 3 and 30 amino acid residues.

The functional group may be engineered to express a bioactive property.

The functional group may be engineered to control or alter charge of the peptide or preparation.

The functional group may be engineered to control or alter pH of the peptide or preparation.

The functional group may be engineered for a target indication.

The target indication may be selected from cell culture, cell delivery, wound healing, treatment of biofilm, and combinations thereof.

The functional group may have a sequence selected from RGD, IKVAV, YIGSR, LKKTETQ, SNKPGVL, PKPQQFFGLM, GKLTWQELYQLKYKGI, and GGG.

The peptide may be configured to self-assemble into a substantially ionically-crosslinked hydrogel.

The peptide may be configured to self-assemble into a shear-thinning hydrogel.

The peptide may be configured to self-assemble into a substantially transparent hydrogel.

The buffer may comprise from about 5 mM to about 200 mM ionic salts.

The ionic salts may dissociate into at least one of sodium, potassium, calcium, magnesium, iron, ammonium, pyridium, quaternary ammonium, chloride, and sulfate ions.

The ionic salts may comprise sodium chloride, ammonium chloride, magnesium chloride, potassium chloride, calcium chloride, ammonium sulfate, magnesium sulfate, sodium sulfate, potassium sulfate, calcium sulfate, sodium bicarbonate, and combinations thereof.

The buffer may comprise from about 10 mM to about 150 mM sodium chloride.

The peptide may have a bacterial endotoxin level of less than about 10 EU/mg.

The preparation may comprise between 0.1% w/v and 8.0% w/v of the peptide.

The preparation may comprise between 0.5% w/v and 6.0% w/v of the peptide.

The preparation may comprise between 0.5% w/v and 3.0% w/v of the peptide.

The preparation may comprise between 0.5% w/v and 1.5% w/v of the peptide.

The preparation may comprise between 0.5% w/v and 1.0% w/v of the peptide.

The preparation may comprise between 0.7% w/v and 2.0% w/v of the peptide.

The preparation may comprise between 0.7% w/v and 0.8% w/v of the peptide.

The hydrogel may comprise between 0.25% w/v and 6.0% w/v of the peptide.

The hydrogel may comprise between 1.5% w/v and 6.0% w/v of the peptide.

The hydrogel may comprise between 0.25% w/v and 3.0% w/v of the peptide.

The peptide may be configured to self-assemble into a hydrogel having between 90% w/v and 99.9% w/v aqueous solution.

The peptide may have a net charge of from +2 to +11.

The peptide may have a net charge of from +5 to +9.

The peptide may be lyophilized.

The preparation may be sterile.

The preparation may be substantially free of a preservative.

The preparation may be thermally stable between −20° C. and 150° C.

The preparation may be sterilized by or autoclave sterilization.

In some embodiments, the preparation is administered as a hemostat, antimicrobial barrier dressing, and/or autolytic debridement agent.

The method may further comprise debridement of the target tissue prior to administration of the preparation.

The method may comprise providing at least one of the preparation and the buffer.

For example, the method may comprise providing the preparation. The method may comprise providing the buffer.

The method may comprise providing at least one of the peptide and the biocompatible solution. For example, the method may comprise providing the peptide. The method may comprise providing the biocompatible solution.

The method may comprise providing at least one of the peptide, the biocompatible solution, and the buffer separately.

The microbial contamination may be a microbial colonization or infection.

The target site may be associated with a wound.

The method may comprise administering the preparation in an amount effective to treat at least one of partial and full thickness wounds (e.g., pressure sores, leg ulcers, diabetic ulcers), first and second degree burns, tunneled/undermined wounds, surgical wounds (e.g., associated with donor sites/grafts, tissue and cell grafts, Post-Moh's surgery, post laser surgery, podiatric, sound dehiscence), trauma wounds (e.g., abrasions, lacerations, burns, skin tears), gastrointestinal wounds (e.g., anal fistulas, diverticulitis, ulcers), and draining wounds.

In accordance with one aspect, there is provided a method of treating biofilm. The method may comprise administering to a target site of the biofilm a thermally stable preparation comprising a purified amphiphilic peptide in an aqueous biocompatible solution.

The peptide may comprise a folding group having a plurality of charged amino acid residues and hydrophobic amino acid residues arranged in a substantially alternating pattern and a turn sequence. The peptide may be configured to self-assemble into a hydrogel. The preparation may be administered in an amount effective to promote deactivation of a target microbial organism associated with the biofilm. The method may comprise administering a buffer to the target site comprising an effective amount of an ionic salt and a biological buffering agent to form the hydrogel.

The disclosure contemplates all combinations of any one or more of the foregoing aspects and/or embodiments, as well as combinations with any one or more of the embodiments set forth in the detailed description and any examples.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A includes schematic and microscopic images of an assembled peptide hydrogel matrix with encapsulated cells as compared to collagen, according to one embodiment;

FIG. 1B includes a schematic drawing of a mixing device and a schematic representation of cells in a hydrogel matrix, according to one embodiment;

FIG. 2 is a graph showing the antimicrobial activity of the peptide hydrogel, in accordance with one embodiment;

FIG. 3 includes images of a mouse model post-burn injury with bacterial infection showing antimicrobial activity of the peptide hydrogel, in accordance with one embodiment;

FIG. 4 includes graphs of corrected optical density (OD) at 625 nm of arginine-rich hydrogels when challenged with bacterial colonies at various cell loading concentrations (CFU/dm²);

FIG. 5 includes exemplary peptide sequences in accordance with several embodiments and graphs showing the antimicrobial activity of the exemplary peptide hydrogels;

FIG. 6 includes microscopic images of cells engrafted on the peptide hydrogels, according to one embodiment;

FIG. 7 is a graph showing hemolytic activity of a 2 wt % peptide hydrogel, according to one embodiment;

FIG. 8 is a microscopic image of a live/dead cellular viability assay;

FIG. 9 includes an image of a mouse model and a magnified image of treated wound on the mouse model, according to one embodiment;

FIG. 10 includes graphs showing quantified MRSA colony forming units (CFU) of tissues treated with peptide preparations, according to one embodiment;

FIG. 11A is a graph showing average percent wound closure of wounds treated with the peptide preparation and control preparations, according to one embodiment;

FIG. 11B is a graph showing percent of treated wound area characterized as sloughy, necrotic, and having granulation, according to one embodiment;

FIG. 12A includes images of wounds treated with the peptide preparation and control preparations, according to one embodiment;

FIG. 12B includes microscopic images of hematoxylin and eosin stained tissues, treated according to one embodiment;

FIG. 12C includes microscopic images of hematoxylin and eosin stained tissues, treated according to one embodiment;

FIG. 13 includes graphs showing inflammation scores of treated tissues, according to one embodiment;

FIG. 14 includes images showing inflammation and granuloma of treated tissues, according to one embodiment;

FIG. 15 includes images of swelling in treated wounds at days 0, 3, and 7, according to one embodiment;

FIG. 16 is a graph of swelling score of treated wounds, according to one embodiment;

FIG. 17 includes images of scarring in treated wounds at days 0, 14, 21, and 25, according to one embodiment;

FIG. 18 is a graph of scarring score of treated wounds, according to one embodiment;

FIG. 19 includes microscopic images of hematoxylin and eosin stained tissues, treated according to one embodiment;

FIG. 20 includes graphs of inflammation score, abscessation score, and lymphoid follicle score of treated wounds, according to one embodiment;

FIG. 21A includes images of treated and untreated tissues, according to one embodiment;

FIG. 21B is a graph of infection score of treated wounds, according to one embodiment;

FIG. 21C is a graph of microbiology data of treated wounds, according to one embodiment;

FIG. 21D is a graph of infection score of treated wounds, according to one embodiment;

FIG. 21E is a graph of microbiology data of treated wounds, according to one embodiment;

FIG. 22A is a graph of static light scattering (SLS) at 266 nm of exemplary peptides as a function of temperature, according to some embodiments;

FIG. 22B is a graph of static light scattering (SLS) at 266 nm of exemplary peptides as a function of temperature, according to some embodiments;

FIG. 23 includes graphs showing absorbance of a peptide hydrogel as a function of peptide concentration, according to one embodiment;

FIG. 24 is a graph showing net charge of a peptide preparation as a function of pH value, according to one embodiment;

FIG. 25 is a visual representation of net peptide charge at a pH of 7.4 for several amino acid residues, according to one embodiment;

FIG. 26A is a graph of microbial proliferation of MRSA after treatment with two formulations of the peptide preparation, according to one embodiment;

FIG. 26B is a graph of microbial proliferation of MRSA after treatment with the peptide preparation or silver gel, according to one embodiment;

FIG. 26C includes images of MRSA and P. aeruginosa inoculated murine wounds before and after treatment with the peptide preparation or silver gel, according to one embodiment;

FIG. 27 includes graphs of crystal violet absorption at 570 nm of biofilm samples, treated with the preparation according to one embodiment;

FIG. 28 includes images of biofilm samples spotted on agar plates and treated with the preparation according to one embodiment;

FIG. 29 includes images of 48-hour aged biofilm, treated according to one embodiment;

FIG. 30 includes images and graphs of pig skin samples inoculated with MRSA or P. aeruginosa PA01 and treated with the preparation according to one embodiment;

FIG. 31A includes images and graphs of cell viability assays treated with the preparation according to one embodiment;

FIG. 31B is a graph of selective antimicrobial activity exhibited by the preparation, according to one embodiment;

FIG. 31C includes images of selective antimicrobial activity exhibited by the preparation, according to one embodiment;

FIG. 32 includes images of sustained therapeutic activity of the administered peptide hydrogel compared to conventional polymer, according to one embodiment;

FIG. 33 is a microscopy image of positively charged peptide hydrogels, according to one embodiment;

FIG. 34 is a photograph of the preparation provided in an end-use container, according to one embodiment FIG. 35A includes images of bacterial colonies after administration of the peptide hydrogel or control, according to one embodiment;

FIG. 35B is a graph of antimicrobial activity of the hydrogel preparation and silver gel, according to one embodiment;

FIG. 35C is a graph of antimicrobial activity against MRSA of the hydrogel preparation, according to one embodiment;

FIG. 35D is a graph of antimicrobial activity against P. aeruginosa of the hydrogel preparation, according to one embodiment;

FIG. 36A is a graph of gram-positive antimicrobial activity of the hydrogel preparation, according to one embodiment;

FIG. 36B is a graph of gram-negative antimicrobial activity of the hydrogel preparation, according to one embodiment;

FIG. 37A is a graph of antimicrobial efficacy of the hydrogel preparation against monomicrobial and polymicrobial biofilms, according to one embodiment; and

FIG. 37B is a graph of antimicrobial efficacy of two formulations of the hydrogel preparation against P. aeruginosa biofilms, according to one embodiment.

### DETAILED DESCRIPTION

The preparation disclosed herein may generally comprise a substantially flowable, intra-tissue antimicrobial wound closure matrix. The preparation may prevent superficial and deep tissue infections by eliminating pathogens through direct cell membrane disruption. The self-assembling peptide hydrogel may generally provide reduction of a broad spectrum of multi-drug resistant organisms (MDRO) through antimicrobial properties, conformation to complex wound geometries through flowable gel properties, reduction and/or control of bleeding through hemostatic properties, and provide a temporary extracellular scaffolding material for tissue repair.

It has been postulated that the skin flora, presence of hematoma, serous fluid, and dead space in superficial surgical incisions may be a source of infection, acting as a culture medium for infectious organisms. Subcutaneous drains may be used to reduce the risk of infection. However, the use of postoperative subcutaneous wound drainage is not universally accepted. The preparations and methods disclosed herein are generally effective at treating wound sites by reducing dead space and fluid collection during the early phase of wound healing, controlling bioburden in contaminated tissues. For example, the flowable intratissue application of a surgical wound matrix may provide such benefits. The preparations and methods disclosed herein may additionally allow for combination use of pre and postoperative modalities.

The beneficial properties of the preparation may enable treatment and prevention of infections, for example, surgical site infections (SSI). Current methods for SSI treatment and prevention include preoperative and perioperative systemic antibiotic use, perioperative application of antibiotic drug-containing (for example, gentamicin, vancomycin) dressings or beads, and strict adherence to operating room sterile technique procedures. The efficacy of systemic antibiotic use is significantly reduced by the requirement to adhere to carefully timed drug delivery due to short half-lives of less than 2 hours. Roughly half of surgeries using systemic antibiotic administration fail to achieve adequate timing preoperatively and/or incorrectly time subsequent dosing.

The use of topical antibiotics has been challenged by the limited efficacy without sustained availability of the drugs at the site of infection due to rapid drug dispersion and potential toxicities of drug-containing materials. Both systemic and topical antibiotic use may be affected by inefficacy against MDROs. Although there have been improvements in prevention of SSI through compliance to strict guidelines and increased surveillance, the risks and challenges continue to exist. These challenges will likely increase with increasing antibiotic resistance.

Treatment of infections is further complicated by the emergence of multi drug resistant organisms (MDRO). For example, as many as 50% of S. aureus isolates may be resistant to methicillin. The failure rate of antibiotics in treating infection is estimated to be in excess of 22%. Conventional antibiotics are becoming progressively less effective with fewer options available to treat wound infections. There are concerns over antibiotic resistance, limited efficacy due to lack of sustained and effective availability of the bioactive molecule at the site of infection, inability to target a broad spectrum of pathogens, and potential toxicity. In accordance with some embodiments, topical antibiotics such as mupirocin, intra-tissue gentamicin collagen sponge and beads, intra-tissue vancomycin may be administered in combination with the preparations and methods disclosed herein.

In particular, abdominal clean-contaminated wounds tend to be prone to higher risk of SSI's. For example, laparotomies carry over three times higher risk of wound infection than other wounds. While methods disclosed herein may refer to treatment of SSI (for example, in abdominal wounds), it should be understood that any surgical or non-surgical wound site may be treated by the methods and preparations disclosed herein.

Treatment options may focus on eliminating bacterial pathogens from wounds, through mechanisms against which bacterial resistance will be unlikely to develop. For example, the direct membrane-disruption mechanisms of the disclosed preparations and methods. Thus, the disclosed preparations and methods may treat, for example, prevent, bioburden in surgical wounds by a broad-spectrum antimicrobial mechanism of action that is capable of disrupting bacterial cell-membranes, substantially locally acting and non-leaching, and/or substantially safe and biocompatible with tissue. The use of the self-assembling peptide preparations described herein in intra-operative surgical care may provide safe and effective elimination of pathogens, thereby reducing, inhibiting, or preventing downstream complications associated with infections.

Preparations comprising self-assembling peptide hydrogels are disclosed herein. The self-assembled peptide may be amphiphilic. The peptide may generally have a folding group having a plurality of charged amino acid residues and hydrophobic residues arranged in a substantially alternating pattern. The peptide may include functional groups to provide desired physical or chemical properties upon administration. The purified peptide may include counterions that improve biocompatibility of the preparation. The counterions may control the self-assembly, physical and chemical properties of the peptide. The counterions may enhance the therapeutic functional properties of the peptide. The preparation may include the peptide in an aqueous biocompatible solution. The preparation may include a buffer solution capable of inducing self-assembly of the peptide upon contact. The buffer solution may contain a buffering agent and ionic salts. The buffer solution composition may be designed to control the assembled hydrogel's physical or chemical properties. The preparation may be designed to be thermally stable.

In general, the preparation may have shear-thinning properties and a substantially physiological pH level. The self-assembled hydrogel may have antimicrobial, antiviral, and/or antifungal properties. The preparation may be administered topically or parenterally. The preparation may be administered for tissue engineering applications. Certain exemplary applications include cell delivery, cell culture, treatment and prevention of fungal infections, treatment and prevention of bacterial infections, wound healing, biofilm treatment, biofilm management, and prevention of biofilm and wound infection, including infection of chronic wounds. Other tissue engineering applications are within the scope of the disclosure.

Methods of administering the preparation to a subject are disclosed herein. The methods may generally include selecting a target site for administration and administering the preparation to the target site. Methods of administering the preparation may also include mixing the preparation with a buffer configured to induce self-assembly of the peptide to form the hydrogel and administering the hydrogel to the target site. In certain exemplary embodiments, the preparation and/or hydrogel may be administered by spray, aerosol, dropper, tube, ampule, instillation, injection, or syringe.

In certain embodiments, methods of administering cells to a subject are disclosed herein. The methods may generally include suspending the cells in a solution comprising a self-assembling peptide and administering an effective amount of the suspension to a target site of the subject. The methods may comprise combining the solution with a buffer configured to induce self-assembly of the peptide. The solution may be combined with the buffer prior to administration, concurrently with administration, or after administration. The buffer may generally comprise an effective amount of an ionic salt and a biological buffering agent.

Unlike other peptides in aqueous solution, the peptides disclosed herein undergo self-assembly. The self-assembly may enable the peptides to be administered in a concentrated or localized manner to a target tissue. For example, self-assembling peptides may be administered at higher concentrations when compared to free floating peptides. The self-assembling peptides may exhibit the clinical benefit of reducing offsite toxicity of the peptides, due to the localizing effect upon administration. Additionally, the therapeutic dosage of peptides may be increased in the vicinity of the target administration site.

Unlike other polymers in aqueous solution, the peptides disclosed herein may undergo self-assembly in situ at the target site. The in situ self-assembly may enable the peptides to be administered to a target tissue and allow to physically or ionically crosslink, for example, within seconds of administration. For example, self-assembling peptides may be administered directly to target site. Conventional free-floating peptides or polymers usually need a crosslinking agent or exogenous added covalent crosslinking agent. Thus, the self-assembling peptides disclosed herein may provide the clinical benefit of reducing product application and complexity. Additionally, the ionic crosslinking of peptides upon self-assembly may provide the benefit of selecting between product removal and permanent adherence to a target administration site.

### Select Definitions

Hydrogels are a class of materials that have significant promise for use in soft tissue and bone engineering. The general characteristic of hydrogels that make them important materials for these applications are their well hydrated, porous structure. Hydrogels may be designed to be compatible with the adhesion and proliferation of various cell types, e.g., fibroblasts and osteoblasts, making them potential tissue engineering scaffolds for generating connective tissue, such as cartilage, tendons, and ligaments, and bone.

The hydrogel material may be cytocompatible. Cytocompatibility, defined herein, means that the hydrogel must not be adverse to desired cells, in vitro and/or in vivo. Adversity to cells may be measured by cytotoxicity, cell adhesion, proliferation, phenotype maintenance, and/or differentiation of progenitor cells.

The hydrogel material may be biocompatible. “Biocompatible,” defined herein, means that a material does not cause a significant immunological and/or inflammatory response if placed in vivo. Biocompatibility may be measured according to International Organization for Standardization (ISO) 10993 standards.

The hydrogel material may be biodegradable affording non-toxic species. The hydrogel material may be proteolytically biodegradable. “Proteolytic” biodegradation, defined herein, refers to local degradation of the material in response to the presence of cell-derived proteases and/or gradual degradation with the proliferation of cells. The hydrogel material may be hydrolytically biodegradable. “Hydrolytic” biodegradation, defined herein, refers to polymer degradation without assistance from enzyme under biologic conditions.

The hydrogel material may be bioresorbable. Bioresorbable, defined herein, means that the hydrogel material breaks down into remnants that are natural products readily absorbed into the body, resulting in complete loss of original mass.

The hydrogel material may be shear-thinning. “Shear-thinning,” as described herein, refers to a variable apparent viscosity, in particular, a decreasing viscosity with increasing applied stress. For instance, the shear-thinning hydrogel may exhibit non-Newtonian fluid properties. In particular the hydrogels disclosed herein may be administered through a needle or catheter and rapidly resume gelation after removal of the mechanical force.

The hydrogel and/or other materials disclosed herein may be referred to as having one or more physiological properties. As disclosed herein, physiological properties or values refer to those which are compatible with the subject. In particular, physiological properties or values may refer to those which are compatible with a particular target tissue. In certain embodiments, physiological properties or values may refer to those which are substantially similar to the properties or values of the target tissue. Physiological properties may include one or more of pH value, temperature, net charge, water content, stiffness, and others.

“Self-assembling” peptides include such peptides which, typically, after being exposed to a stimulus, will assume a desired secondary structure. The peptides may self-assemble into a higher order structure, for example a three-dimensional network and, consequently, a hydrogel. The self-assembled hydrogel may contain peptides in a tertiary and/or quaternary structure through charge screening, hydrophobic, and disulfide interactions. Peptides have been observed to self-assemble into helical ribbons, nanofibers, nanotubes and vesicles, surface-assembled structures and others. Self-assembling peptides may assemble responsive to certain environmental conditions, e.g., pH, temperature, net charge, exposure to light, applied sound wave, or presence or absence of environmental factors. The environmental conditions may occur upon administration to a subject or by combination with a buffer. In other embodiments, the peptide may assemble spontaneously in solution under neutral pH level. The peptide may assemble spontaneously in solution under physiological conditions and/or in the presence of a cation and/or anion.

The self-assembling peptides may assemble into an alpha helix, pi-helix, beta sheet, random coil, turn, beta pleated parallel, antiparallel, twist, bulge, or strand connection secondary structure and combinations of thereof. For example, a 20 amino acid peptide which self-assembles into β-strands may comprise alternating valine and lysine residues flanking a tetrapeptide sequence (-VDPPT). When dissolved in low ionic strength and buffered aqueous solution, the exemplary peptide resides in an ensemble of random coil conformers due to electrostatic repulsions of the positively charged lysine residues. Upon increasing the ionic strength and/or pH of the solution, the lysine-based positive charge is relieved due to either screening of the charge or deprotonating a sufficient amount of the side chain amines. This exemplary action enables peptide folding into an amphiphilic β-hairpin. In the folded state, the exemplary peptide self-assembles via lateral and facial associations of the hairpins to form a non-covalently crosslinked hydrogel containing β-sheet rich fibrils. Thus, the self-assembling peptides may be designed to undergo hydrogelation under varying conditions through rational design of the peptide sequence.

The self-assembling peptides disclosed herein may assemble into a nano-porous tertiary structure. As disclosed herein, the nano-porous structure is a three-dimensional matrix containing pores having an average size of 1-1000 nm. The pores or voids may constitute between 10% and 90% of the three-dimensional matrix by volume. For example, the pores or voids may constitute 10%, 20%, 30%, 40%, 50%, 60%, 70%, 80%, or 90% of the three-dimensional matrix by volume. The pores may be permeable and allow diffusion of liquid and/or gas. The nano-porous structure is constructed by physical crosslinks, allowing ionic bonds to be broken and reformed upon asserted stress. These nano-porous structure may allow for cells to attach and/or migrate through the matrix. The nano-porous structure may also mimic the endogenous extra-cellular matrix environment of tissues and, optionally, be selected to mimic a specific tissue.

“Disassembly” of the peptides may refer to the ability of the peptide to assume a lower order structure after being exposed to a stimulus. Disassembly may also refer to the ability of the physically crosslinked peptide to temporarily break hydrophobic and disulfide bonds to assume a lower order structure after being exposed to a stimulus. For example, a tertiary structure protein may disassemble into a secondary structure protein, and further disassemble into a primary structure peptide. In accordance with certain embodiments, self-assembly and disassembly of the peptide may be reversible.

Preparations and formulations disclosed herein may generally be referred to as peptide preparations. The peptide preparations may include a self-assembling peptide and/or a self-assembled hydrogel as disclosed herein. The peptide preparation may include a cytocompatible and/or biocompatible solution. The preparation may include a buffer. While reference is made to a solution, it should be understood that the preparation may be in the form of a liquid, gel, or solid particle. In certain embodiments, for example, the preparation may be in the form of the assembled hydrogel. In other embodiments, the preparation may be in the form of a lyophilized powder.

The peptide preparation may further include one or more bioactive components for tissue engineering, such as, functionalized peptides, cells, media, serum, collagen and other structure-imparting components, antibodies and antigens, bioactive small molecules, and other bioactive drugs. “Bioactivity” as described herein refers to the ability of a compound to impart a biological effect.

Cell containing preparations and formulations disclosed herein may be referred to as cell suspensions. Cell suspensions include a plurality of cells, e.g., living cells, suspended in a solution. The solution may be or comprise water, media, or buffer. The suspension may generally further comprise a self-assembling peptide and/or a self-assembled hydrogel, as disclosed herein. While reference is made to cells, it should be understood that the suspension may contain cell fragments and/or tissue, e.g. tissue grafts, in addition to or instead of the cells. For example, the suspension may contain live or dead cells or cell fragments, spheroids, and/or cell aggregates.

The cells may be isolated from living tissue and subsequently maintained and/or grown in cell culture. The cell culture conditions may vary, but generally include maintaining the cells in a suitable vessel with a substrate or medium that supplies the essential nutrients, e.g., amino acids, carbohydrates, vitamins, minerals, growth factors, hormones, and gases, e.g., CO₂ and O₂, and regulating the physio-chemical environment, e.g., pH, osmotic pressure, temperature. The cells may be maintained in live cell lines, e.g., a population of HeLa cells descended from a single cell and containing the same genetic makeup.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, “treatment” of an injury, condition, or disease refers to reducing the severity or frequency of at least one symptom of that injury, condition, or disease, compared to a similar but untreated subject. Treatment can also refer to halting, slowing, or reversing the progression of an injury, condition, or disease, compared to a similar but untreated subject. Treatment may comprise addressing the root cause of the injury, condition, or disease and/or one or more symptoms. “Management” of an injury, condition, or disease may refer to reducing the severity or frequency of at least one symptom of that injury, condition, or disease, to a tolerable level, as determined by the subject or a health care provider.

As used herein an effective amount refers to a dose sufficient to achieve a desired result. For example, the effective amount may refer to a concentration sufficient to achieve self-assembly of the hydrogel and/or provide desired properties. An effective amount may refer to a dose sufficient to prevent advancement, or to cause regression of an injury, condition, or disease, or which is capable of relieving a symptom of an injury, condition, or disease, or which is capable of achieving a desired result. An effective amount can be measured, for example, as a concentration of peptide or other component in the preparation, solution, or buffer. An effective amount can be measured, for example, as a concentration of bioactive agent or an effect or byproduct of a bioactive agent. An effective amount can be measured, for example, as a number of cells or number of viable cells, or a mass of cells (e.g., in milligrams, grams, or kilograms), or a volume of cells (e.g., in mm³).

Throughout this disclosure, formulation may refer to a composition or preparation or product.

Administered “in combination,” as used herein, means that two (or more) different treatments are delivered to the subject during the course of the subject's affliction with the injury, e.g., the preparation is delivered with a second agent after the subject has been diagnosed with the condition or injury and before the condition or injury has been cured or eliminated. In certain embodiments, administration in combination means the preparation additionally comprises one or more second agent. In some embodiments, the delivery of one treatment is still occurring when the delivery of the second begins, so that there is overlap. This is sometimes referred to herein as “simultaneous” or “concomitant” or “concurrent delivery.” In other embodiments, the delivery of one treatment ends before the delivery of the other treatment begins. This is sometimes referred to herein as “successive” or “sequential delivery.”

In embodiments of either case, the treatment is more effective because of combined administration. For example, the second agent is a more effective, e.g., an equivalent effect is seen with less of the second agent, or the second agent reduces symptoms to a greater extent, than would be seen if the second agent were administered in the absence of the preparations disclosed herein, or the analogous situation is seen with the preparation. In some embodiments, delivery is such that the reduction in a symptom, or other parameter related to the disorder is greater than what would be observed with one treatment delivered in the absence of the other. The effect of the two treatments can be partially additive, wholly additive, or greater than additive (i.e., synergistic). The delivery can be such that an effect of the administration of the preparation is still detectable when the second agent is delivered. In some embodiments, one or more treatment may be delivered prior to diagnosis of the patient with the injury.

As used herein, a subject may include an animal, a mammal, a human, a non-human animal, a livestock animal, or a companion animal. The term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the disclosure includes all vertebrates, for example, non-mammals (such as birds, for example, chickens; amphibians; reptiles) and mammals, such as non-human primates, domesticated, and agriculturally useful animals, for example, sheep, dog, cat, cow, pig, rat, among others. The term “non-human animals” includes research animals, for example, for example, mouse, rat, rabbit, dog, cat, pig, among others.

### Properties of the Peptide Sequence and Secondary Structure

The peptides disclosed herein may have a sequence configured to fold into a desired secondary structure. The secondary structure may refer to a three-dimensional form of local segments of proteins. The secondary structure may comprise, for example, pleated sheet, helical ribbon, nanotube and vesicle, surface-assembled structure, and others. The peptides disclosed herein may have a sequence configured to self-assemble into a desired tertiary structure. The tertiary structure may refer to a three-dimensional organization of secondary structure protein forms. The tertiary structure may comprise, for example, three-dimensional matrix, porous matrix, nano-porous matrix.

Self-assembling peptides disclosed may be designed to adopt a secondary, for example, β-hairpin, and/or tertiary structure in response to one or more signals. Typically, after adopting the secondary structure, the peptides will self-assemble into a higher order structure, e.g., a hydrogel. In certain embodiments, the self-assembly does not take place unless side chains on the peptide molecules are uniquely presented in the secondary structure conformation. The self-assembling peptides may assemble responsive to certain environmental conditions, e.g., pH, temperature, net charge, exposure to light, applied sound wave, or presence or absence of environmental factors. The environmental conditions which induce self-assembly may occur upon administration to a subject, e.g., upon contact with a target tissue. In some embodiments, the environmental conditions which induce self-assembly may occur upon combination of the peptide preparation with a buffer configured to induce self-assembly. The buffer may have a pH or composition configured to induce self-assembly. For example, the buffer may have a concentration of ions configured to induce self-assembly.

Self-assembly of the peptides disclosed herein may produce compact structures that exhibit biophysical structural relationships with the intended function of the peptide. For example, a compact tertiary structure may have a higher number of active amino acid residues per unit area, compared to unassembled peptides. In the particular example of antimicrobial peptides, the tertiary structure may enable a higher concentration of charged, e.g., positively charged, amino acid residues per area, increasing antimicrobial properties (e.g., bacterial membrane destabilization and disruption).

In certain embodiments, the self-assembling peptide hydrogels may include those disclosed in and/or prepared by the methods disclosed in any of U.S. Pat. Nos. 8,221,773; 7,884,185; 8,426,559; 7,858,585; and 8,834,926, incorporated herein by reference in their entireties for all purposes. For example, the self-assembling peptide hydrogels may be or comprise any of SEQ ID NOS: 1-20 from U.S. Pat. Nos. 8,221,773, 7,884,185, and 7,858,585; and SEQ ID NOS: 1-33 from U.S. Pat. No. 8,834,926. Other self-assembling peptides are known and may be employed to bring about the methods disclosed herein.

The desired properties of the self-assembling peptides may be controlled by peptide design. The self-assembling peptides may be small peptides, e.g., from about 6 to about 200 residues or from about 6 to about 50 residues or from about 10 to about 50 residues. Any of the amino acid residues may be a D isoform. Any of the amino acid residues may be an L isoform.

Self-assembling peptides disclosed herein may be designed to be substantially amphiphilic when assembled into the tertiary structure. “Amphiphilic” molecules, e.g., macromolecules or polymers, as disclosed herein, typically contain hydrophobic and hydrophilic components. Peptide amphiphiles are one exemplary class of amphiphilic molecules. Peptide amphiphiles are peptide-based molecules that typically have the tendency to self-assemble into high-aspect-ratio nanostructures under certain conditions. The exemplary conditions may comprise selected pH, temperature, and ionic strength values. One particular type of peptide amphiphiles comprise alternating charged, neutral, and hydrophobic residues, in a repeated pattern, for example, as disclosed herein. A combination of intermolecular hydrogen bonding and hydrophobic and electrostatic interactions may be designed to form well-defined self-assembled nanostructures by assembly of the disclosed peptide amphiphiles.

The self-assembling peptides may include additional amino acids, for example, an epitope. For example, the self-assembling peptides may include additional functional groups, optionally selected by peptide design. Exemplary functional groups disclosed herein comprise a biologically derived motif, for example, having an effect on biological processes such as cell signal transduction, cell adhesion in the extra-cellular matrix (ECM), cell growth, and cell mobility. The peptide may include one or more modifications, for example, a linker or spacer. In some embodiments, at least one of the N-terminus and the C-terminus may be modified. For example, at least one of the N-terminus and the C-terminus may be amidated. At least one of the N-terminus and the C-terminus may be acetylated. In certain exemplary embodiments, the C-terminus may be amidated and/or the N-terminus may be acetylated. In some embodiments, at least one of the N-terminus and the C-terminus may be free.

In general, the self-assembling peptides may have a folding group configured to adopt the secondary and/or higher order structure. Exemplary self-assembling peptides may have a folding group designed to adopt a β-hairpin secondary structure. Exemplary self-assembling peptides may have a folding group designed to adopt a three-dimensional nano-porous matrix tertiary structure. Self-assembling peptides disclosed herein may be designed to adopt a β-hairpin secondary structure and/or nano-porous matrix tertiary structure in response to one or more environmental stimulus at the target site, e.g., at a topical or parenteral site. The self-assembling peptides may also be designed to self-assemble into a range of other self-assembled structures, such as spherical micelles, vesicles, bilayers (lamellar structures), nanofibers, nanotubes, and ribbons.

The self-assembly folding group may have between about 2 and about 200 residues, for example, between about 2 and about 50 residues, between about 10 and about 30 residues, between about 15 and about 25 residues, for example, about 20 residues.

In accordance with some embodiments, the self-assembling folding group may include hydrophobic amino acids. “Hydrophobic” amino acid residues are those which tend to repel water. Such hydrophobic amino acids may include glycine, alanine, valine, leucine, isoleucine, proline, phenylalanine, methionine, threonine, and tryptophan. In certain embodiments, the hydrophobic amino acid residues may comprise valine.

The folding group may be functionalized by addition of other functional residues as described herein, or conserved for self-assembly. Exemplary functional residues include basic, neutral, aliphatic, aromatic, and polar amino acid residues.

The folding group may have a plurality of basic, neutral, aliphatic, aromatic, polar, charged amino acid residues. The folding group may have a plurality of hydrophobic amino acid residues arranged in a substantially alternating pattern with non-hydrophobic amino acid residues. In certain embodiments, the folding group may have a plurality of hydrophobic amino acid residues arranged in a substantially alternating pattern with a plurality of charged amino acid residues.

The folding group may comprise a turn sequence. The turn sequence may include one or more internal amino acid residues within the folding group. In certain embodiments, the turn sequence may be substantially centrally located within the folding group.

The turn sequence may have between about 2 and about 20 residues, for example, between about 2 and about 10 residues, between about 2 and about 8 residues, between about 2 and about 5 residues, for example, about 2 residues, about 3 residues, about 4 residues, or about 5 residues.

In exemplary embodiments, the turn sequence may include one or more of proline, aspartic acid, threonine, and asparagine. The turn sequence may include D-proline and/or L-proline. In some embodiments, the turn sequence may have 1-4 proline residues, for example, 1 proline residue, 2 proline residues, 3 proline residues, or 4 proline residues.

Exemplary self-assembling peptides may have a folding group sequence comprising [AY]_{N}[T][YA]_{M}, where A is 1-3 amino acids selected from one or more of basic, neutral, aliphatic, aromatic, polar, and charged amino acids, Y is 1-3 hydrophobic amino acids, T is 2-8 turn sequence amino acids, and N and M are each independently between 2 and 10. Y amino acids may independently be selected from glycine, alanine, valine, leucine, isoleucine, proline, phenylalanine, methionine, threonine, and tryptophan. In some embodiments, the folding group sequence may be Y[AY]_{N}[T][YA]_{M}Y-NH₂.

Certain exemplary self-assembling peptides may have a folding group sequence comprising [XY]_{N}[T][YX]_{M}, where X is 1-3 charged amino acids, Y is 1-3 hydrophobic amino acids, T is 2-8 turn sequence amino acids, and N and M are each independently between 2 and 10. X amino acids may independently be selected from arginine, lysine, tryptophan, and histidine. Y amino acids may independently be selected from glycine, alanine, valine, leucine, isoleucine, proline, phenylalanine, methionine, threonine, and tryptophan. In some embodiments, the folding group sequence may be Y[XY]_{N}[T][YX]_{M}Y-NH₂.

Certain exemplary self-assembling peptides may have a folding group sequence comprising [ZY]_{N}[T][YZ]_{M}, where Z is 1-3 polar or charged amino acids, Y is 1-3 hydrophobic amino acids, T is 2-8 turn sequence amino acids, and N and M are each independently between 2 and 10. Z amino acids may independently be selected from glutamine, asparagine, histidine, serine, threonine, tyrosine, cysteine, alanine, valine, leucine, isoleucine, proline, phenylalanine, arginine, lysine, aspartic acid, and glutamic acid. Y amino acids may independently be selected from glycine, alanine, valine, leucine, isoleucine, proline, phenylalanine, methionine, threonine, and tryptophan. In some embodiments, the folding group sequence may be Y[ZY]_{N}[T][YZ]_{M}Y-NH₂.

One exemplary self-assembling peptide may have a folding group comprising -RYRYRYTYRYRYR- where R is an arginine residue, Y is 1-3 hydrophobic amino acids, and T is 2-6 turn sequence amino acids.

One exemplary self-assembling peptide may have a folding group comprising -VXVXVXVXVTXVXVXVXV- where V is a valine residue, X may be independently selected from charged and neutral amino acid residues serine, glutamic acid, lysine, tryptophan, and histidine, and T is 2-8 turn sequence amino acids. In some embodiments, the exemplary folding group may comprise a series of hydrophobic valine amino acid residues alternating with independently selected hydrophilic and/or neutral amino acid residues.

One exemplary self-assembling peptide may have a folding group comprising -KYKYKYTYKYKYK- where R is an arginine residue, Y is 1-3 hydrophobic amino acids, and T is 2-6 turn sequence amino acids.

One exemplary self-assembling peptide may have a folding group comprising -VZVZVZVTVZVZVZV- where V is a valine residue, Z is 1-3 hydrophilic amino acids, and T is 2-6 turn sequence amino acids.

Exemplary self-assembling peptides may have a turn sequence comprising 2-8 turn sequence amino acids, for example 2-5 turn sequence amino acids. The turn sequence amino acids may be selected from proline, for example D-proline and/or L-proline, aspartic acid, and asparagine. In some embodiments, the turn sequence may be (d)PP, (d)PG, or NG.

Exemplary self-assembling peptides having a turn sequence include VKVRVRVRV(d)PPTRVRVRVKV-NH₂ and VLTKVKTKV(d)PPTKVEVKVLV-NH₂. In the exemplary peptides, the tetrapeptide turn sequence (—V(d)PPT-) was selected to adopt a type II′ turn and positioned within the middle of the peptide sequence. This four-residue turn sequence occupies the i, i+1, i+2 and i+3 positions of the turn. The heterochiral sequence ((d)P (i+1)−P (i+2)) dipeptide was selected for its tendency to adopt dihedral angles consistent with type II′ turns. Incorporation of a bulky β-branched residue (valine) at the i position of the turn sequence enforces the formation of a trans prolyl amide bond at the i+1 position. The placement of valine at this position is selected to design against the formation of a cis prolyl bond, which results in β-strands that adopt an extended conformation rather than the intended hairpin. Threonine exhibits a statistical propensity to reside at the i+3 position. Therefore, threonine was selected to be incorporated at this position within the tetrapeptide sequence, which bears a side-chain hydroxyl group capable of hydrogen bonding to the amide backbone carbonyl at the i position, to further stabilize the turn.

The exemplary folding peptides may be designed to include high propensity j-sheet forming residues flanking the type II′ turn sequence. The selection of alternation of hydrophobic and hydrophilic residues along the strands provides an amphiphilic j-sheet when the peptide folds. For example, lysine may be chosen as a hydrophilic residue to provide a side chain pKa value of about 10.5. Side chain amines are generally protonated when dissolved under slightly acid conditions, forming unfavorable electrostatic interactions between β-strands of the hairpin and inhibiting peptide folding and self-assembly. However, as pH is increased to about pH 9, a sufficient portion of the lysine side chains become deprotonated allowing the peptide to fold into an amphiphilic β-hairpin. The electrostatic interactions may be employed to design pH responsiveness of the disclosed peptides.

While not wishing to be bound by theory, it is believed the amphiphilic β-hairpin is stabilized in the intramolecular folded state by van der Waals contacts between neighboring amino acid side chains within the same hairpin. The formation of intramolecular hydrogen bonds between cross β-strands of the hairpin and the propensity for the turn sequence to adopt at type II′ turn may further stabilize the folded conformation. Once in the folded state, the lateral and facial associations of the β-hairpins may be selected to design self-assembly. For example, lateral association of β-hairpins promotes the formation of intermolecular hydrogen bonds and van der Waals contacts between neighboring amino acids.

Exemplary self-assembling peptides may have a folding group sequence comprising (X)a(Y)b(Z)c-[(d)PP, (d)PG, or NG]-(Z)c(Y)b(X)a, where the turn sequence is (d)PP, (d)PG, or NG, (d)P is a D-proline, X is a charged amino acid, Y is a hydrophobic amino acid, Z is a hydrophobic amino acid or a polar amino acid, and a, b, and c are each independently an integer from 1-10. In certain embodiments, X is independently selected from valine, leucine, isoleucine, phenylalanine, tryptophan, tyrosine, threonine, and combinations thereof. In certain embodiments, Y is independently selected from glutamic acid, serine, alanine, proline, aspartic acid, and combinations thereof. In some embodiments, Z is independently selected from glutamine, glutamic acid, lysine, arginine, and combinations thereof.

Exemplary self-assembling peptides may have a folding group sequence comprising (Z)c(Y)b(X)a-[(d)PP, (d)PG, or NG]-(X)a(Y)b(Z)c, where the turn sequence is (d)PP, (d)PG, or NG, (d)P is a D-proline, X is a charged amino acid, Y is a hydrophobic amino acid, Z is a hydrophobic amino acid or a polar amino acid, and a, b, and c are each independently an integer from 1-10. In certain embodiments, X is independently selected from valine, leucine, isoleucine, phenylalanine, tryptophan, tyrosine, threonine, and combinations thereof. In certain embodiments, Y is independently selected from glutamic acid, serine, alanine, proline, aspartic acid, and combinations thereof. In some embodiments, Z is independently selected from glutamine, glutamic acid, lysine, arginine, and combinations thereof.

Any of the charged, hydrophobic, polar, or amphipathic amino acids disclosed herein may derive one or more of their properties from the composition of the biocompatible solution.

Hydrophobic amino acids are those which tend to repel water. Hydrophobic amino acids include alanine, valine, leucine, isoleucine, proline, tyrosine, tryptophan, phenylalanine, methionine, and cysteine. The hydrophobic amino acids may be independently selected from alanine, valine, leucine, isoleucine, proline, phenylalanine, methionine, cysteine, and combinations thereof. In some embodiments, the hydrophobic amino acids comprise valine.

Charged amino acids are those which tend to have an electric charge under the given conditions. Charged amino acids may have side chains which form salt bridges. Charged amino acids include alanine, valine, leucine, isoleucine, proline, phenylalanine, cysteine, arginine, lysine, histidine, aspartic acid, and glutamic acid. The folding group may comprise 2-10 charged amino acids, for example 2-8 charged amino acids.

The charged amino acids may be positively charged amino acids. The folding group may comprise 2-10 charged amino acids, for example 2-8 charged amino acids. The positively charged amino acids may be independently selected from arginine, lysine, histidine, and combinations thereof. The folding group may comprise 2-8 arginine residues, lysine residues, or a combination of arginine and lysine residues. In some embodiments, the folding group may comprise 6 positively charged residues selected from arginine, lysine, or a combination of arginine and lysine.

The charged amino acids may be negatively charged amino acids. The folding group may comprise 2-10 negatively charged amino acids, for example, 2-8 negatively charged amino acids. In some embodiments, the negatively charged amino acids may be independently selected from aspartic acid, glutamic acid, and combinations thereof.

Polar amino acids are those which have an uneven charge distribution. Polar amino acids may tend to form hydrogen bonds as proton donors or acceptors. Polar amino acids include glutamine, asparagine, histidine, serine, threonine, tyrosine, and cysteine.

Amphipathic amino acids are those which have both a polar and non-polar component. Amphipathic amino acids may be found at the surface of proteins or lipid membranes. Amphipathic amino acids include tryptophan, tyrosine, and methionine.

Exemplary self-assembling peptides may have a folding group sequence of any of SEQ ID NOS: 1-23. The self-assembling peptide may have a folding group sequence of SEQ ID NO: 1. In certain embodiments, the self-assembling peptide may have a folding group sequence of SEQ ID NO: 2. The self-assembling peptide may have a folding group sequence of SEQ ID NO: 3. The self-assembling peptide may have a folding group sequence of SEQ ID NO: 4. The self-assembling peptide may have a folding group sequence of SEQ ID NO: 5. The self-assembling peptide may have a folding group sequence of SEQ ID NO: 6. The self-assembling peptide may have a folding group sequence of SEQ ID NO: 7. The self-assembling peptide may have a folding group sequence of SEQ ID NO: 8. The self-assembling peptide may have a folding group sequence of SEQ ID NO: 9. The self-assembling peptide may have a folding group sequence of SEQ ID NO: 10. The self-assembling peptide may have a folding group sequence of SEQ ID NO: 11. The self-assembling peptide may have a folding group sequence of SEQ ID NO: 12. The self-assembling peptide may have a folding group sequence of SEQ ID NO: 13. The self-assembling peptide may have a folding group sequence of SEQ ID NO: 14. The self-assembling peptide may have a folding group sequence of SEQ ID NO: 15. The self-assembling peptide may have a folding group sequence of SEQ ID NO: 16. The self-assembling peptide may have a folding group sequence of SEQ ID NO: 17. The self-assembling peptide may have a folding group sequence of SEQ ID NO: 18. The self-assembling peptide may have a folding group sequence of SEQ ID NO: 19. The self-assembling peptide may have a folding group sequence of SEQ ID NO: 20. The self-assembling peptide may have a folding group sequence of SEQ ID NO: 21. The self-assembling peptide may have a folding group sequence of SEQ ID NO: 22. The self-assembling peptide may have a folding group sequence of SEQ ID NO: 23.

Exemplary self-assembling peptides which have shear-thinning properties include VKVRVRVRV(d)PPTRVRVRVKV-NH₂, and VKVRVRVRV(d)PPTRVEVRVKV-NH₂ (which has a single substitution of glutamic acid at position 15 on the hydrophilic face). The glutamic acid substitution results in a faster rate of gelation of the self-assembling peptide gel in the presence of ionic salts in the biocompatible solution. The negatively charged glutamic acid lowers the overall positive charge of the peptide and enables faster folding and self-assembly.

Exemplary self-assembling peptides which have shear-thinning properties that can be tuned for net peptide charge include VKVRVRVRV(d)PPTRVEVRVKV-NH₂, and VKVKVKVKV(d)PPTKVEVKVKV-NH₂, (which has an arginine substituted for lysine on the hydrophilic face). The lysine substitution lowers the peptide net charge at physiological pH that allows for better mammalian cell cytocompatibility when compared to peptides with high arginine content (higher net charge). The exemplary peptides are antimicrobial self-assembling peptides.

Exemplary self-assembling peptides which have shear-thinning properties that can be tuned for peptide gels with faster rate of gelation and increased stiffness include FKFRFRFRV-(d)PPTRFRFRFKF-NH₂, (which has valine substituted for phenylalanine on the hydrophobic face). The phenylalanine substitution increases the hydrophobic face of the peptide that allows for stiffer and faster gelation of peptide gels. The exemplary peptides are antimicrobial self-assembling peptides.

Exemplary self-assembling peptides which have shear-thinning properties include enantiomer forms of the exemplary sequences listed above, such as an enantiomer form of VKVRVRVRV(d)PPTRVRVRVKV-NH₂, (d)V(d)K(d)V(d)R(d)V(d)R(d)V(d)R(d)V(L)P (d)P(d)T(d)R(d)V(d)R(d)V(d)R(d)V(d)K(d)V-NH₂, (which has D isoforms of the sequence and an L isoform of P). The isoform substitution may provide control of peptide degradation and increased stability without compromising peptide net charge at physiological pH. The sequence may provide better compatibility with mammalian cells. The peptide may be a complete enantiomer (as shown above) or a partial enantiomer such that any one or more of the amino acids may be an enantiomer of the sequences listed above. The exemplary peptides are antimicrobial self-assembling peptides. The self-assembling peptide may comprise at least one guanidine moiety. The guanidine moiety may be associated with an organic molecule which makes up part of the peptide chain. In exemplary embodiments, a guanidine group may be incorporated as part of the side chain of an arginine residue. However, the peptide may comprise guanidine moieties which are not associated with arginine residues.

Other exemplary self-assembling peptides include Ac-VEVSVSVEV(d)PPTEVSVEV EVGGGGRGDV-NH₂ and VEVSVSVEVdPPTEVSVEVEV-NH₂.

A guanidine moiety is generally a highly polar group which, when positioned on a cationic peptide, may allow for pairing with hydrophobic and hydrophilic groups forming salt bridges and hydrogen bonds. Such a peptide may display a high capacity to penetrate cell membranes and provide antimicrobial activity. The guanidine moiety may also promote peptide stability by improving peptide folding, physical characteristics and thermal stability of the peptide and/or hydrogel.

The peptide may generally have 20-50% guanidium content, as measured by number of guanidine groups by total number of amino acid residues of the peptide. For instance, an exemplary peptide sequence having 20 amino acid residues, of which 6 are arginine residues having a guanidine group, has 30% guanidium content. The exemplary peptides may penetrate and disrupt cell membranes.

### Properties of the Peptide Hydrogel Preparation

The preparation may generally comprise the self-assembling peptide in a biocompatible solution. For example, the peptide may be dissolved or substantially dissolved in the biocompatible solution. The preparation may comprise between about 0.1% w/v and about 8.0% w/v of the peptide. The preparation may be formulated for a target indication. For instance, the concentration of the self-assembling peptide may be selected based on a target indication. For example, an exemplary preparation having antimicrobial properties may comprise less than 1.5% w/v of the peptide, for example, between about 0.5% w/v of the peptide and 1.0% w/v of the peptide.

Exemplary preparations may comprise between about 0.25% w/v and about 6.0% w/v of the peptide, for example, between about 0.5% w/v and about 6.0% w/v of the peptide. When the peptide is purified, the preparation may comprise up to about 6.0% w/v of the peptide. In certain embodiments, the preparation may comprise less than about 3.0% w/v of the peptide, for example, between about 0.25% w/v and about 3.0% w/v of the peptide, between about 0.25% w/v and about 2.0% w/v of the peptide, between about 0.25% w/v and about 1.25% w/v of the peptide, or between about 0.5% w/v of the peptide and about 1.5% w/v of the peptide. The preparation may comprise between about 0.5% w/v and about 1.0% w/v of the peptide, between about 0.7% w/v and about 2.0% w/v of the peptide, or between about 0.7% w/v and about 0.8% w/v of the peptide. For instance, the preparation may comprise about 0.25% w/v, about 0.5% w/v, about 0.7% w/v, about 0.75% w/v, about 0.8% w/v, about 1.0% w/v, about 1.5% w/v of the peptide, about 2.0% w/v, or about 3.0% w/v. In particular embodiments, the preparation may comprise less than about 1.5% w/v of the peptide. The preparation may comprise less than about 1.25% w/v of the peptide or less than about 1.0% w/v of the peptide. In one exemplary embodiment, the preparation may comprise about 0.75% w/v of the peptide.

After combination with the buffer, the hydrogel may have between about 0.05% w/v and 6.0% w/v of the peptide. For example, the hydrogel may have between about 0.1% w/v, and 6.0% w/v of the peptide, between about 0.25% w/v and 6.0% w/v of the peptide, between about 1.5% w/v and 6.0% w/v of the peptide, between about 0.25% w/v and 3.0% w/v of the peptide, between about 0.25% w/v and 1.0% w/v of the peptide, between about 0.25% w/v and 0.5% w/v of the peptide, or between about 0.3% w/v and 0.4% w/v of the peptide. The peptide preparation and buffer may be combined to form the hydrogel at a ratio of between about 2:1 to 0.5:1 peptide preparation to buffer. In some embodiments, the peptide preparation and buffer may be combined to form the hydrogel at a ratio of about 1:1.

The peptides in the preparation may be purified. As disclosed herein, “purified” may refer to compositions treated for removal of contaminants. In particular, the purified peptides may have a composition suitable for clinical application. For example, the peptides may be purified to meet health and/or regulatory standards for clinical administration. The peptide may be at least 80% purified, for example, at least 85%, at least 90%, at least 92%, at least 95%, at least 98%, at least 99%, or at least 99.9%.

In certain embodiments, the peptides may be purified to remove or reduce residual organic solvent content from solid phase synthesis of the peptides. The peptide may comprise less than 20% residual organic solvent by weight. The peptide may comprise less than 15% residual organic solvent by weight. The peptide may comprise less than 10% residual organic solvent by weight. For example, the peptide may comprise less than 8%, less than 5%, less than 2%, less than 1%, or less than 0.1% residual organic solvent by weight. Exemplary organic solvents which may be removed or reduced from the synthesized peptide include trifluoroacetic acid (TFA), acetonitrile, isopropanol, N,N-Dimethylformamide, triethylamine, Ethyl Ether, and acetic acid.

The purified peptides may be substantially free of Trifluoroacetic acid (TFA). For example, the purified peptides may have less than 1% w/v residual TFA, or between about 0.005% w/v and 1% w/v residual TFA.

The purified peptide may be substantially free of acetonitrile. In some embodiments, the purified peptide may have less than about 410 ppm residual acetonitrile. The purified peptide may have between about 0.005 ppm and about 410 ppm residual acetonitrile.

The purified peptide may be substantially free of isopropanol. In some embodiments, the purified peptide may have less than about 400 ppm residual isopropanol. The purified peptide may have less than about 100 ppm residual isopropanol. The purified peptide may have between about 0.005 ppm and 100 ppm residual isopropanol.

The purified peptide may be substantially free of N,N-Dimethylformamide. In some embodiments, the purified peptide may have less than about 880 ppm residual N,N-Dimethylformamide. The purified may have between about 0.005 ppm and about 880 ppm residual N,N-Dimethylformamide.

The purified peptide may be substantially free of triethylamine. In some embodiments, the purified peptide may have less than about 5000 ppm residual triethylamine.

The purified peptide may have between about 0.005 ppm and about 5000 ppm residual triethylamine.

The purified peptide may be substantially free of Ethyl Ether. In some embodiments, the purified peptide may have less than about 1000 ppm residual Ethyl Ether. The purified peptide may have between about 0.005 ppm and about 1000 ppm residual Ethyl Ether.

The purified peptides may be substantially free of acetic acid. For example, the purified peptides may have less than 2% w/v residual acetic acid, for example, less than 1% w/v residual acetic acid, less than 0.5% w/v residual acetic acid, less than 0.1% w/v residual acetic acid, between about 0.0001% w/v and 2% w/v residual acetic acid, or between about 0.005% w/v and 0.1% w/v residual acetic acid.

In general, the purified peptide and/or biocompatible solution may have properties consistent with regulatory limits defined by the International Council for Harmonization of Technical Requirements for Pharmaceuticals for Human Use (ICH).

The biocompatible solution of the preparation may refer to a substantially liquid carrier for the peptide. The biocompatible solution may generally be an aqueous solution. The biocompatible solution may comprise water, for example, deionized water. Deionized water may have a resistivity of greater than about 18 MΩ and a conductivity of less than about 0.056 μS at 25° C. Deionized water may have a maximum endotoxin specification of 0.03 endotoxin units (EU)/ml and 1 CFU/mL microbial action or less. Deionized water may have a total organic carbon (TOC) concentration of 10 ppb or less. The biocompatible solution may comprise pharmaceutical grade water. Pharmaceutical grade water may have 500 ppb total organic carbon (TOC) or less and 100 CFU/ml microbial action or less. The biocompatible solution may comprise injection grade water. Injection grade water may have a maximum endotoxin specification of 0.25 endotoxin units (EU)/ml and 10 CFU/10 ml microbial action or less. In certain embodiments, the preparation or biocompatible solution may be substantially free of chloride ions.

The preparation or peptide may comprise counterions. As disclosed herein, a counterion may refer to a charge balancing ion. The preparation or peptide may have an effective amount of counterions to render the preparation substantially electrically neutral. The preparation or peptide may have an effective amount of counterions to render the preparation substantially biocompatible and/or stable. The preparation or peptide may have an effective amount of counterions to control repulsion of anionic or cationic residues of the peptide. The concentration of counterions may be dependent on the peptide sequence and concentration of the peptide and any additives. In exemplary embodiments, the peptide may comprise between 0.1-20% counterions. Additionally, the charge of the counterions may be dependent on the charge of the peptide and any additives. Thus, the counterions may be anions or cations. In general, the counterions may be cytocompatible. In certain embodiments, the counterions may be biocompatible. For instance, the counterions may comprise acetate, citrate, ammonium, fluoride, or chloride. In other embodiments, the preparation or peptide may be substantially free of chloride counterions.

In exemplary embodiments, the preparation or peptide may comprise an effective amount of acetate counterions. In particular, preparations having a peptide concentration which comprises residual TFA may have an amount of acetate counterions sufficient to balance the residual TFA. Briefly, TFA is commonly used to release synthesized peptides from solid-phase resins. TFA is also commonly used during reversed-phase HPLC purification of peptides. However, residual TFA or fluoride may be toxic and undesirable in peptides intended for clinical use. Furthermore, TFA may interact with the free amine group at the N-terminus and side chains of positively charged residues (for example, lysine, histidine, and arginine). The presence of TFA-salt counterions in the peptide preparation may be detrimental for biological material and may negatively affect the accuracy and reproducibility of the intended peptide activity.

TFA-acetate salt exchange by acetate or hydrochloride may be employed to counteract some or all of the negative effects of TFA described above. The inventors have determined the acetate counterion is surprisingly well suited for maintaining biological activity of the peptide preparation and for controlling solubility of the peptide and charge for self-assembly of the peptide. Furthermore, acetic acid (pKa=4.5) is weaker than both trifluoroacetic acid (pKa about 0) and hydrochloric acid (pKa=−7). Acetate counterions may additionally control pH of the peptide preparations to be physiologically neutral.

The preparation may have variable hydrogelation kinetics. In accordance with certain embodiments, the hydrogelation kinetics of the preparation may be designed for a particular mode of administration. The preparation may be administered as a liquid. The preparation may be administered as a solid or semi-solid. The preparation may be administered as a gel. The preparation may be administered as a combination of hydrogel suspended in a liquid. The preparation may have a variable apparent viscosity. For instance, the preparation may have an apparent viscosity effective to allow injection under the conditions of administration. In certain embodiments, the preparation may have an apparent viscosity which decreases with increasing shear stress.

The preparation may be configured to reversibly self-assemble and disassemble in response to applied stress, for example, applied mechanical force. The solid or gel preparation may become disrupted with increasing applied stress, to be later restored once the applied stress is reduced. The solid or gel may become fluid in response to applied stress, for example, during delivery through a delivery device. The peptide may be capable of undergoing sequential phase transitions in response to applied stress. The peptide may be capable of recovering after each one or more sequential phase transitions.

The preparation may be configured to reversibly self-assemble and disassemble responsive to at least one of change in temperature, change in pH, contact with an ion chelator, dilution with a solvent, applied sound wave, lyophilization, vacuum drying, and air drying. The administered fluid may conform to tissue voids before reforming as a solid or gel. Thus, the solid or gel preparation may be injectable, flowable, or sprayable under the appropriate shear stress. Once administered, the preparation may be restored to a solid or gel form, substantially conforming to the target site. The formation may occur within less than a minute, about one minute, less than about 2 minutes, less than about 3 minutes, less than about 5 minutes, or less than about 10 minutes. The formation may occur within about one minute, less than about 30 seconds, less about 10 seconds, or about 3 to 5 seconds.

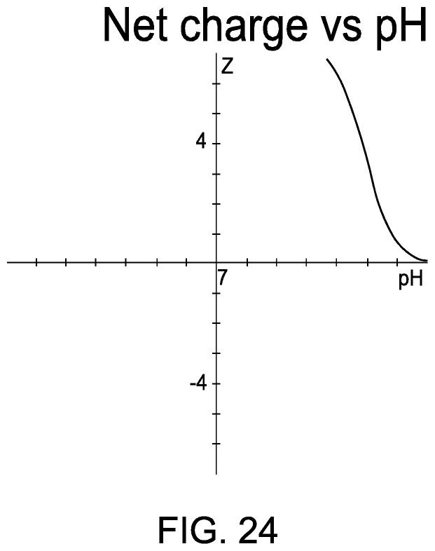

The peptide may be purified. For example, the peptide may be lyophilized. As shown in FIG. 24, net charge may be quantified as a function of pH value. The exemplary peptide measured in FIG. 24 is an arginine-rich peptide having two lysine residues. The exemplary peptide of FIG. 24 has a net charge of +9 at a pH of 7. Other peptides are within the scope of the disclosure. For example, the purified peptide may have a net charge between −9 to +11 at pH 7, for example, −7 to +9 at pH 7. As disclosed herein, “net charge” may refer to a total electric charge of the peptide as a biophysical and biochemical property, typically as measured at a pH of 7.

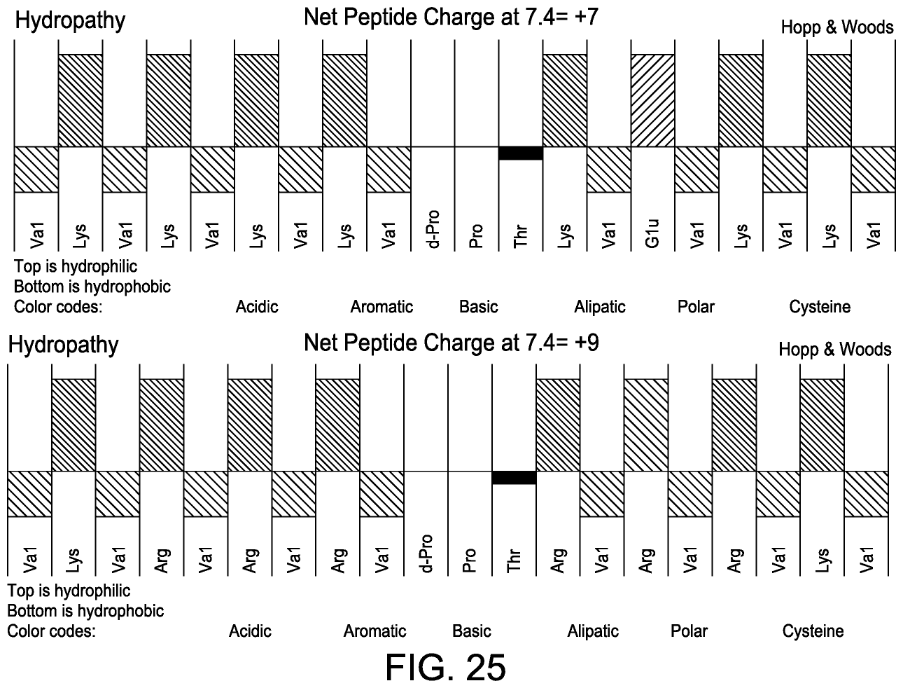

The purified peptide may have a net charge of from −7 to +11 at pH 7. In some embodiments, the peptide may have a net charge of from +2 to +9, for example, +5 to +9 or +7 to +9. The purified peptide may have a charge of about +5, +6, +7, +8, +9, +10, or +11 at pH 7. Exemplary peptides having a charge of +5 to +9 include VLTKVKTKV(d)PPTKVEVKVLV, VKVRVRVRV(d)PPTRVRVRVKV, and VKVRVRVRV(d)PPTRVEVRVKV. In other embodiments, the purified peptide may be substantially neutral. In other embodiments, the peptide may have a net negative charge. An exemplary peptide having a net negative charge is VEVSVSVEV(d)PPTEVSVEVEV. As shown in FIG. 25, a single substitution of glutamic acid in the peptide sequence may alter net peptide charge from +7 (top panel) to +9 (bottom panel) at pH 7, as well as alter isoelectric point from 11.45 to 14. Net charge may be selected by peptide design. Design of electrostatic charge in the peptide hydrogel may allow control of charge interaction with cell membrane and proteins.

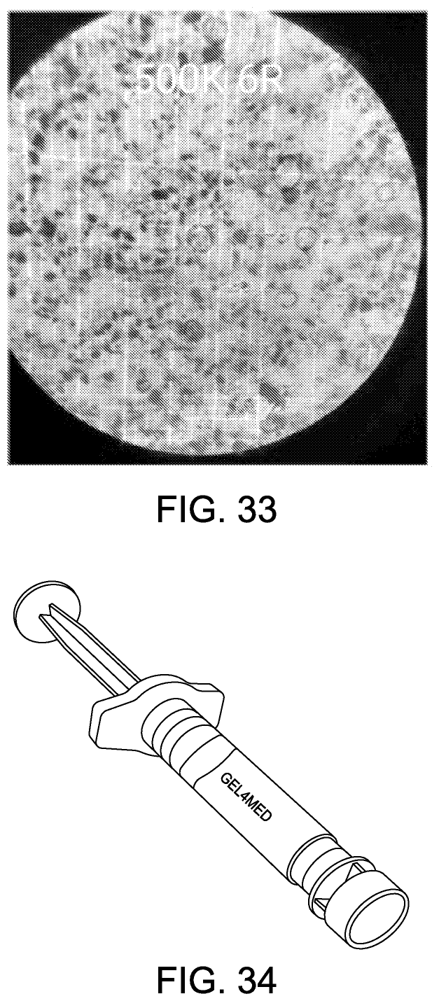

The peptide may be designed to have a charge that adsorbs and/or promotes deactivation of proteins at a target site of administration. For instance, positively charged peptide hydrogels may promote adsorption of negatively and neutrally charged molecules such as small molecules, proteins, and extravesicular membranes. Negatively charged peptide hydrogels may promote adsorption of positively and neutrally charged molecules such as small molecules, proteins, and extravesicular membranes. Furthermore, the peptide may be designed to have regions of positive, neutral, or negative charge, to varying degrees. In certain embodiments, the peptide charge may be designed such that when placed into a rich solution of charged molecules, the peptide may soak out or absorb the molecules into the hydrogels attaching the molecules to the peptides by adsorption. FIG. 33 is a microscopy image showing negatively charged Trypan blue adsorbed on a positively charged hydrogel.

The purified peptide may have greater than 70% w/v, greater than 80% w/v, or greater than 90% w/v nitrogen, for example, between 70% w/v and 99.9% w/v nitrogen.

The purified peptide may have a bacterial endotoxin level of less than about 10 EU/mg, for example, less than about 5 EU/mg, less than about 2 EU/mg, or less than about 1 EU/mg. In other embodiments, the purified peptide may have an endotoxin level of between about −0.010 to −0.015 EU/ml. For instance, the purified peptide may have an OD at 410 nm of between 0.004 to 0.008, for example, about 0.006. The peptide hydrogel may have an OD at 410 nm of between 0.010 to 0.020, for example, about 0.015. In some embodiments, the purified peptide and/or preparation may be substantially free of endotoxins.

The purified peptide in the biocompatible solution may have a water content of between about 1% w/v and about 20% w/v, for example, at least about 10% w/v, or less than about 15% w/v.

The purified peptide may have an isoelectric point of between about 7-14. For example, the purified peptide may have an isoelectric point of about 7, 8, 9, 10, 11, 12, 13, or 14.

The purified peptide may be configured to self-assemble into a hydrogel having a shear modulus of between about 2 Pa to 3500 Pa as determined by rheology testing. For example, the purified peptide may self-assemble into a hydrogel having a shear modulus of greater than 100 Pa, between 100 Pa and 3500 Pa, between 100 Pa and 3000 Pa, between 2 Pa and 1000 Pa, or between 2 Pa and 500 Pa. For example, a formulation having 0.75% w/v peptide may have a shear modulus of between about 2 Pa and 500 Pa. A formulation having 1.5% w/v peptide may have a shear modulus of between about 100 Pa and 3000 Pa. A formulation having 3.0% w/v peptide may have a shear modulus of between about 1000 Pa and 10000 Pa. Thus, shear modulus of the hydrogel may be controlled by selection of peptide concentration in the formulation.

The peptide may be designed to adopt a predetermined secondary structure. For example, the peptide may be designed to adopt a β-hairpin secondary structure, as previously described. The predetermined secondary structure may comprise a structure preselected from at least one of a β-sheet, an α-helix, and a random coil. In exemplary embodiments, the hydrophobic amino acid residues (for example, quantity, placement, and/or structure of the hydrophobic amino acid residues) may be selected to self-assemble the peptide into a polymer having a majority of β-sheet structures. In particular embodiments, the hydrophobic amino acid residues may be selected to control stiffness of the hydrogel. For example, an amount and type of hydrophobic amino acid residues may be selected to control stiffness of the hydrogel.

In some embodiments, an external stimulus such as temperature, change in pH, light, and applied sound wave may be used to control and promote preferential secondary structure formation of the self-assembling peptide. Control of the secondary structure formation may enhance biological, biophysical, and chemical therapeutic functions of the peptide. For example, higher cell membrane penetration of self-assembling peptides may be achieved by exposing β-hairpin peptides to high pH (for example, at least pH 9) or high temperatures (for example, at least 125° C.) or low temperatures (for example, 4° C. or lower). The result is hydrogels with a peptide secondary structure having a majority β-sheet or α-helix formation.

The peptide may be designed to give the preparation shear-thinning properties. In particular, the peptide may be designed to be injectable. For instance, the peptide may be designed to be an injectable solid or gel by employing shear-thinning kinetics. The preparation, in the form of a solid or gel prior to application, may be configured to shear-thin to a flowable state under an effective shear stress applied during administration by the delivery device. In some exemplary embodiments, the solid or gel may shear-thin to a flowable state during injection or topical application with a syringe. Other modes of administration may be employed. The solid or gel may shear-thin to a flowable state during endoscopic administration. The solid or gel may be configured to shear-thin to flow through an anatomical lumen, for example, an artery, vein, gastrointestinal tract, bronchus, renal tube, genital tract, etc. In some embodiments, the shear thinning properties may be employed during transluminal procedures. The peptide may be designed to be sprayable. For example, the peptide may be designed for administration as a spray or other liquid droplet, for example, other propelled liquid droplet, by employing shear-thinning kinetics, as previously described.

The shear-thinning kinetics of the hydrogel may be engineered by altering the net charge of the peptides. In some embodiments, the net charge may be altered by controlling one or more of the presence or absence of cationic particles or peptides, the presence or absence of anionic particles or peptides, buffers, salts, peptide concentration, peptide purity, and the presence or absence of peptide counterions. In particular, shear-thinning may be controlled by altering the peptide purity to achieve the desired shear-thinning kinetics. The net charge of the peptide may be positive. The net charge of the peptide may be negative.

Shear-thinning may be induced by mechanical agitation to the hydrogel or environmental stimulus. Mechanical agitation may be induced, for example, through delivery or sonication mixing. Environmental stimulus may be induced by addition of heat, light, ionic agents, chelator agents, buffers, or proteins, or altering pH level.

Thus, the preparations may be substantially flowable. The methods may include dispensing the preparation through a cannula or needle. The methods may include conformally filling wound beds of any size and shape. The peptide hydrogels may have shear-thinning mechanical properties. The shear-thinning mechanical properties may allow the gel network to be disrupted and become a fluid during administration, for example, during injection from a needle or administration with a spray nozzle. When the applied stress ceases, the gel network may reform and the elastic modulus may be restored within a predetermined period of time, for example, several minutes. The shear-thinning peptide hydrogels may be employed to protect cells from damage during injection, showing an improved viability over direct injection in saline or media. The shear-thinning hydrogel may display non-Newtonian fluid flow, which may allow for effective mixing of excipients, for example, within minutes to a couple hours. In some embodiments, dyes, small molecules, and large molecules may be substantially homogeneously dispersed within the hydrogel in less than 120 minutes, for example, between 30-120 minutes.

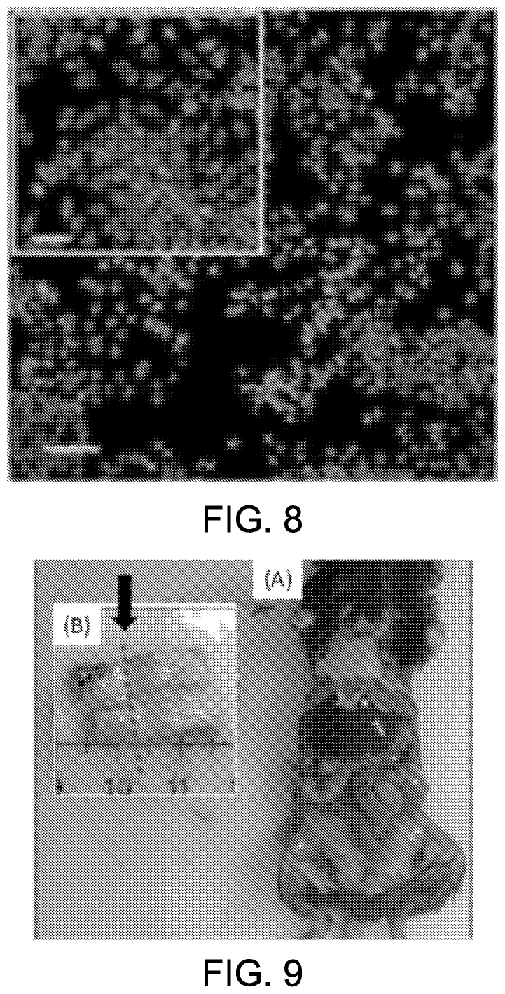

The peptide may self-assemble into a translucent hydrogel. In some embodiments, the peptide may self-assemble into a substantially transparent hydrogel. The transparency of the hydrogel may enable a user or healthcare provider to view surrounding tissues through the hydrogel. In exemplary embodiments, a surgical procedure may be performed without substantial obstruction of view by the hydrogel. The hydrogel may have at least about 70%, at least about 80%, at least about 90%, at least about 95%, at least about 99%, or about 100% light transmittance. The hydrogel may be colorless. The light transmittance and color of the hydrogel may be engineered by tuning the sequence of the peptide and/or the composition of the preparation or solution. As shown in the graphs of FIG. 8, transparency of the peptide hydrogels may be quantified by absorbance measurements. The exemplary peptide hydrogels measured in FIG. 8 are substantially transparent.

In some embodiments, the preparation may include a dye. The dye may be a food-grade dye or a pharmaceutical-grade dye. The dye may be cytocompatible. The dye may be biocompatible. In general, the dye may assist the user or healthcare provider to view the hydrogel after application. The preparation may include an effective amount of the dye to provide a desired opacity of the hydrogel. The hydrogel may comprise an effective amount of the dye to have a light transmittance of less than about 60%, less than about 50%, less than about 40%, less than about 30%, less than about 20%, or less than about 10%. The hydrogel may be substantially opaque when including the dye.

The peptide may self-assemble into a substantially ionically-crosslinked hydrogel. “Ionic crosslinkage” may refer to ionic bonds between peptides to form secondary structure proteins and/or between secondary structure proteins that form the hydrogel tertiary structure. The shear-thinning properties of the hydrogel may be enabled by physical crosslinks, allowing ionic bonds to be broken and reformed. In accordance with certain embodiments, the hydrogel is formed of a majority of ionic crosslinks. For example, more than 50%, more than 60%, more than 70%, more than 80%, more than 90%, more than 95%, more than 99%, or substantially all of the physical crosslinks of the formed hydrogel may be ionic in nature.

The preparation and/or assembled hydrogel may be designed to have a substantially physiological pH level. The preparation or hydrogel may have a pH level of between about 4.0 and 9.0. In some embodiments, the preparation or hydrogel may have a pH level of between about 7.0 and 8.0. The preparation or hydrogel may have a pH level of between about 7.3 and 7.5. The substantially physiological pH may allow administration of the preparation at the time of gelation. In some embodiments, the hydrogel may be prepared at a point of care. The methods may comprise mixing the preparation with a buffer configured to induce self-assembly, optionally agitating the mixture, and administering the preparation or hydrogel at a point of care. The administration may be topical or parenteral, as described in more detail below.

The peptide may be designed to self-assemble in response to a stimulus. The stimulus may be an environmental stimulus, e.g., change in temperature (e.g., application of heat), exposure to light, change in pH, applied sound waves, or exposure to ionic agents, chelator agents, or proteins. The stimulus may be mechanical agitation, e.g., induced through delivery, sonication, or mixing. In some embodiments, the methods may comprise administering the preparation as a liquid. The methods may comprise administering the preparation as a gel. The methods may comprise administering the preparation as a solid or semi-solid.

In some embodiments, the preparation may be designed to self-assemble after a lapsed period of time. For example, the preparation may be designed such that the peptide is configured to begin self-assembly in less than about 5 minutes, in less than about 3 minutes, in less than about 2 minutes, in less than about 30 seconds, in less than about 10 seconds, or in less than about 3 seconds. In certain embodiments, the preparation may be designed such that the peptide is configured to self-assemble, i.e. be substantially self-assembled, within about 60 minutes, within about 30 minutes, within about 15 minutes, within about 10 minutes, within about 5 minutes, within about 3 minutes, within about 2 minutes, within about 30 seconds, within about 10 seconds, within about 5 seconds, or within about 3 seconds. The preparation may have a composition configured to control timing of self-assembly. For example, the preparation may have a composition configured for timed release of ionic agents or pH-altering agents. In certain embodiments, the sequence or structure of the peptide may be designed to control self-assembly of the peptide.

In some embodiments, the methods may comprise combining the preparation with a buffer. The “buffer” may refer to an agent configured to induce gelation, prior to, subsequently to, or concurrently with administration of the preparation to the subject. Thus, in some embodiments, the preparation may comprise a buffer. For example, the preparation may comprise or be combined with up to about 1000 mM of the buffer. The buffer may comprise an effective amount of ionic salts and a buffering agent, for example, to induce gelation and/or provide desired properties. For example, the buffer may be formulated to control or maintain pH of the preparation.

In particular embodiments, the buffer may have an effective amount of ionic salts to control stiffness of the hydrogel. The “ionic salt” may refer to a compound which dissociates into ions in solution. The buffer may comprise between about 5 mM and 400 mM ionic salts. For example, the buffer may comprise between about 5 mM and 200 mM ionic salts, between about 50 mM and 400 mM ionic salts, between about 50 mM and 200 mM ionic salts, or between about 50 mM and 100 mM ionic salts. The ionic salt may be one that dissociates into at least one of sodium, potassium, calcium, magnesium, iron, ammonium, pyridium, quaternary ammonium, chloride, citrate, acetate, and sulfate ions. The ionic salts may comprise sodium chloride, ammonium chloride, magnesium chloride, potassium chloride, calcium chloride, ammonium sulfate, magnesium sulfate, sodium sulfate, potassium sulfate, calcium sulfate, sodium bicarbonate, and combinations thereof.

In exemplary embodiments, the buffer may comprise between about 1 mM and about 200 mM sodium chloride. For example, the buffer may comprise between about 10 mM and about 150 mM sodium chloride, for example between about 50 mM and about 100 mM sodium chloride.

The buffer may comprise counterions. The buffer may have an effective amount of counterions to render the hydrogel substantially electrically neutral. The buffer may have an effective amount of counterions to induce self-assembly of the peptide. The concentration of counterions may be dependent on the composition of the peptide preparation. Additionally, the charge of the counterions may be dependent on the charge of the peptide preparation. Thus, the counterions may be anions or cations. In general, the counterions may be cytocompatible. In certain embodiments, the counterions may be biocompatible. For instance, the counterions may comprise acetate or chloride. In other embodiments, the biocompatible solution may be substantially free of chloride counterions.

The buffer may comprise from about 1 mM to about 150 mM of a biological buffering agent. For example, the buffer may comprise from about 1 mM to about 100 mM of a biological buffering agent, from about 1 mM to about 40 mM of a biological buffering agent, or from about 10 mM to about 20 mM of a biological buffering agent. The biological buffering agent may be selected from Bis-tris propane (BTP), 4-(2-hydroxyethyl)-1-piperazineethanesulfonic acid (HEPES), Dulbecco's Modified Eagle Medium (DMEM), tris(hydroxymethyl)aminomethane (TRIS), 2-(N-Morpholino)ethanesulfonic acid hemisodium salt, 4-Morpholineethanesulfonic acid hemisodium salt (MES), 3-(N morpholino)propanesulfonic acid (MOPS), and 3-(N-morpholino)propanesulfonic acid (MOBS), Tricine, Bicine, (tris(hydroxymethyl)methylamino)propanesulfonic acid (TAPS), N-(2-Acetamido)-2-aminoethanesulfonic acid (ACES), β-Hydroxy-4-morpholinepropanesulfonic acid, 3-Morpholino-2-hydroxypropanesulfonic acid (MOPSO), (N,N-bis(2-hydroxyethyl)-2-aminoethanesulfonic acid) (BES) and combinations thereof. Other biological buffering agents are within the scope of the disclosure.

In exemplary embodiments, the buffer may comprise from about 1 mM to about 150 mM of BTP. The buffer may comprise from about 10 mM to about 100 mM BTP, for example, from about 10 mM to about 50 mM BTP, from about 10 mM to about 40 mM, from about 20 mM to about 40 mM, or from about 20 mM to about 40 mM.

The buffer may additionally comprise at least one of water, an acid, and a base. The acid and/or base may be added in an amount effective to control pH of the buffer to be a substantially physiological pH. In other embodiments, the buffer may be acidic, alkali, or substantially neutral. The buffer may be selected to control pH of the hydrogel and maintain a desired pH at the target site. For example, to control pH of the hydrogel to be a substantially physiological pH at the target site. Thus, the properties of the buffer may be selected based on the target site. The buffer may have additional properties as selected, for example, net charge, presence or absence of additional proteins, etc. The buffer may additionally comprise one or more minerals.

The preparation may further comprise an effective amount of a mineral clay. The preparation may comprise between about 0.1% w/v to about 20% w/v of the mineral clay. For example, the preparation may comprise 0.75% w/v, 1.5% w/v, 2% w/v, 3% w/v, 4% w/v, 8% w/v, 10% w/v, or 20% w/v of the mineral clay. The amount of the mineral clay may be effective to provide desired rheological properties for the target site of application. The amount of the mineral clay may be effective to form a film. The mineral clay may be natural or synthetic. The mineral clay may comprise at least one of laponite and montmorillonite. In some embodiments, the preparation may comprise from a 1:1 to 1:2 ratio (w/v) of the peptide to mineral clay. For example, the ratio of peptide to mineral clay in the preparation may be 1:1, 3:4, 3:8, or 1:2 (w/v).

The preparation may be formulated for a target indication. For instance, the preparation may be formulated for treatment of a microbial infection or inhibition of proliferation of a microorganism, such as a pathogenic microorganism. The preparation may be formulated for treatment of a fungal infection or inhibition of proliferation of a fungal organism. The preparation may be formulated for cell culture and/or cell delivery. The preparation may be formulated for treatment or inhibition of a wound, such as a chronic wound, or biofilm. The preparation may be formulated by engineering the peptide as described in more detail below. The preparation may be formulated by selecting the biocompatible solution and/or additives.

In certain embodiments, the preparation may be formulated for a combination treatment. The preparation may include at least one active agent configured to provide a combination treatment. In some embodiments, the preparation may exhibit synergistic results with combination of the active agent. The active agent may be, for example, an antibacterial composition, an antiviral composition, an antifungal composition, an anti-tumor composition, an anti-inflammatory composition, a hemostat, a cell culture media, a cell culture serum, an anti-odor composition, an analgesic, local anesthetic, or a pain-relief composition. The preparation may be formulated for administration in conjunction with one of the aforementioned compositions. The preparation may be formulated for simultaneous or concurrent combination administration. The preparation may be formulated for sequential combination administration.

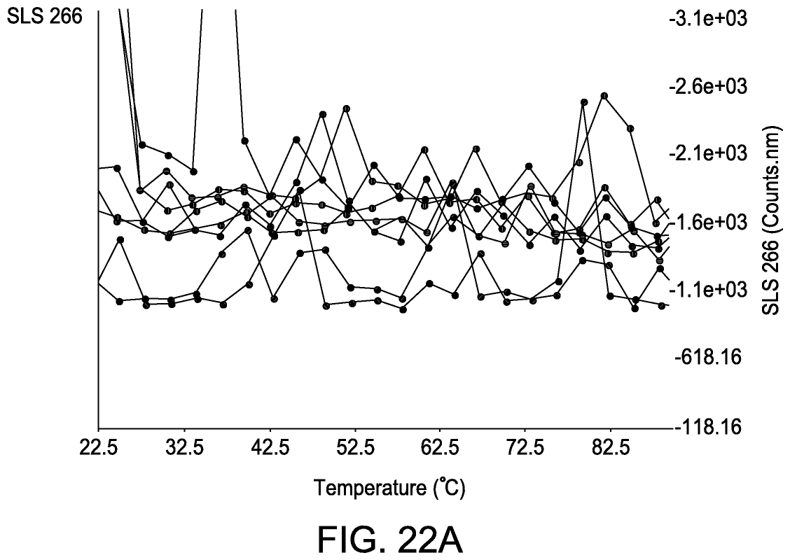

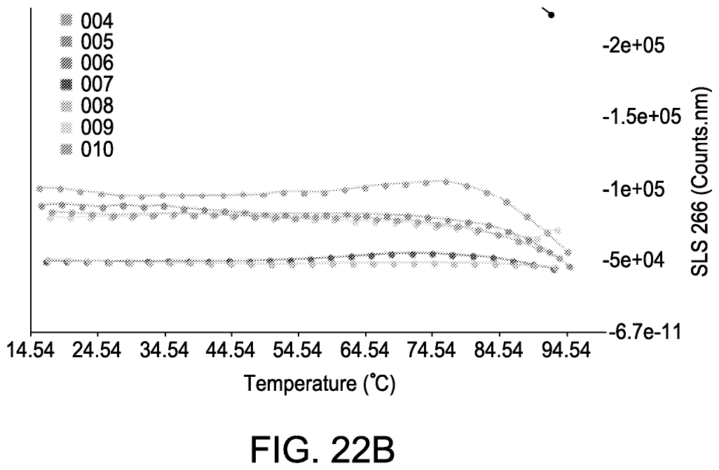

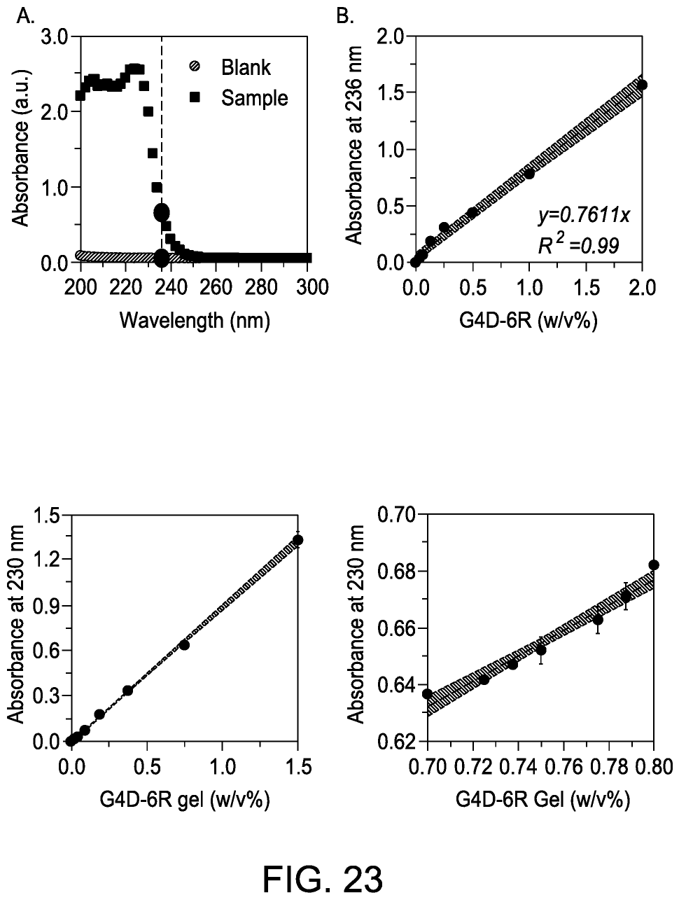

In some embodiments, the preparation and/or hydrogel may be designed to be thermally stable between −20° C. and 150° C., between −20° C. and 125° C., between −20° C. and 100° C., between 2° C. and 125° C., and between 37° C. and 125° C. As disclosed herein, “thermal stability” refers to the ability to withstand temperature treatment without substantial degradation, loss of biological activity, or loss of chemical activity. The graphs of FIGS. 22A-22B show peptide aggregation as measured by static light scattering (SLS) at 266 nm of exemplary peptides as a function of temperature. The exemplary peptides include arginine, lysine, valine, threonine, and proline residues. As shown in the graphs of FIGS. 22A-22B, the peptide hydrogels and peptides are thermostable as a function of temperature.

In certain embodiments, the preparation and/or peptide may be mechanically stable. For instance, the preparation may be shear thinned or sonicated. The preparation may be sonicated without substantial degradation, loss of biological or chemical activity. The preparation may be shear thinned without substantial degradation, loss of biological or chemical activity.

In certain embodiments, the preparation and/or peptide may be sterile or sterilized. The preparation and/or peptide may be sterilized by autoclave sterilization. During autoclave sterilization, the preparation and/or peptide may be heated to a temperature of between 120° C. to 150° C., for example, up to 125° C., up to 135° C., or up to 150° C. The preparation and/or peptide may be held at autoclave temperature for at least about 2 minutes, for example, between about 2-20 minutes or between about 10-160 minutes. The autoclave sterilization may be sufficient to sterilize at least about 90%, 95%, 99%, 99.9%, 99.99%, 99.999%, or 100% of any pathogenic microorganism. The preparation and/or peptide may remain stable during and after autoclave sterilization. For instance, the preparation and/or peptide may remain physically, chemically, biologically, and/or functionally stable after autoclave sterilization.

In certain embodiments, the preparation and/or peptide may be pasteurized. During pasteurization, the preparation and/or peptide may be heated to a temperature of between 50° C. to 100° C., for example, up to 60° C., up to 70° C., or up to 100° C. The preparation and/or peptide may be held at pasteurization temperature for at least about 15 seconds, for example, between about 1-30 minutes or between about 3-15 minutes. The pasteurization may be sufficient to sterilize at least about 90%, 95%, 99%, 99.9%, 99.99%, or 99.999% of any pathogenic microorganism.

In certain embodiments, the preparation may be sterilized by ultra-high temperature (UHT) or high temperature/short time (HTST) sterilization. During UHT or HTST sterilization, the preparation and/or peptide may be heated to a temperature of between 100° C. to 150° C., for example, up to 130° C., up to 140° C., or up to 150° C. The preparation and/or peptide may be held at UHT or HTST temperature for at least about 15 seconds, for example, between about less than 1 minute to about 6 minutes, for example, between about 2-4 minutes. The UHT or HTST sterilization may be sufficient to sterilize at least about 90%, 95%, 99%, 99.9%, 99.99%, or 99.999% of any pathogenic microorganism.

In certain embodiments, the sterilization or pasteurization may be terminal. Terminal sterilization or pasteurization may refer to treatment of the preparation in a sealed end-use package.

The preparation and/or peptide may be stable during and after heat treatment. As disclosed herein, stability during and after heat treatment, for example, autoclave sterilization, may refer to reduced or inhibited degradation, biological activity, and chemical activity. For instance, the preparation and/or peptide may be heat treated without degradation, a loss of biological activity, or a loss of chemical activity. Biological activity may refer to any bioactive property of the peptide disclosed herein. In some embodiments, biological activity may refer to antimicrobial activity. Chemical activity may refer to any chemical or physicochemical property of the peptide disclosed herein. In some embodiments, chemical activity may refer to the ability to self-assemble and or shear-thinning properties of the peptide disclosed herein. Thus, the preparation and/or peptide may be heat treated without loss of antimicrobial activity, self-assembly, or shear-thinning properties. In certain embodiments, heat treatment may enhance one or more biological activity or chemical activity of the peptide and/or preparation. For example, heat treatment may enhance antimicrobial activity, self-assembly, or shear-thinning properties of the peptide or preparation.

The preparation may be sterile. For example, the preparation may remain substantially sterile without the addition of a preservative. The preparation may be substantially sterile without gamma irradiation treatment. The preparation may be substantially sterile without electron beam treatment.

The preparation may have a predetermined shelf-life. “Shelf-life” may refer to the length of time for which the preparation may remain stable and/or maintain efficacy after storage under the given conditions. The preparation and/or hydrogel may have a shelf-life of at least about 1 year at a temperature between −20° C. and 150° C. For instance, the preparation and/or hydrogel may have a shelf-life of at least about 1 year at room temperature (between about 20° C. and 25° C.). The preparation and/or hydrogel may have a shelf-life of at least about 2 years, about 3 years, about 4 years, about 5 years, or about 6 years at room temperature. The preparation and/or hydrogel may be stable at a pressure of up to about 25 psi, for example, up to about 15 psi.

The peptide may be capable of self-assembly at a temperature between 2° C. and 40° C. For example, the peptide may be capable of self-assembly in an environment having a temperature between 2° C. and 20° C., between 20° C. and 25° C., or between 36° C. and 40° C.

The peptide may be substantially unassembled at temperatures higher than 40° C. For instance, the peptide preparation may be substantially liquid at temperatures between 40° C. and 150° C. The peptide preparation may be substantially liquid and thermally stable at temperatures between 40° C. and 125° C. or up to 150° C. Temperature may be controlled for handling of the preparation. For example, the preparation may be heated to a temperature greater than 40° C. for packaging, handling, and/or administration in a liquid state.

The preparation may be formulated for a desired route of administration. For example, the preparation may be formulated for topical or parenteral administration. In particular, the preparation may be engineered to have a viscosity appropriate for topical administration or parenteral administration. Preparations for topical administration may be formulated to withstand environmental and mechanical stressors at the site of administration or target site. Preparations for parenteral administration may be formulated to reduce migration from the site of administration or target site. In other embodiments, preparations for parenteral administration may be formulated to trigger migration from a site of administration to the target site. The preparation may be formulated for administration by a particular delivery device. For example, the preparation may be formulated for administration by spray, dropper, or syringe. The preparation may be formulated for administration by injection or catheter.

Table 1 includes the analytical characterization of three exemplary peptide preparation samples. The exemplary peptides have arginine-rich sequences comprising two lysine amino acid residues. The values were detected by conventional detection methods. Components indicated “N.D.” were below detection limit. Peptide purification, residual solvents, peptide content, and water content, may be selected to control antimicrobial activity and cell membrane disruption potential of the hydrogels.

TABLE 1
Exemplary Peptide Preparations

| | | | | | | Residual Solvents | | | | | | | | |
|---|---|---|---|---|---|---|---|---|---|---|---|---|---|---|
| | | | | | | | | | | N,N- | | | | |
| | | | | | Net | | | | | Dimethyl- | Ethyl | | | |
| | Purity | Acetate | TFA | Water | Peptide | Isopropanol | Methanol | Acetonitrile | Triethylamine | formamide | ether | | | |
| # | (%) | (%) | (%) | (%) | (%) | (ppm) | (ppm) | (ppm) | (ppm) | (ppm) | (ppm) | DCM | DIC | MPS |
| 1 | 97.9 | 19.3 | N.D. | 6.5 | 78.6 | 81 | 28 | 17 | N.D. | N.D. | N.D. | N.D. | N.D. | N.D. |
| 2 | 93.7 | 16.3 | N.D. | 7.1 | 79.3 | 63 | 28 | 18 | 56 | 116 | N.D. | N.D. | N.D. | N.D. |
| 3 | 96.0 | 16.9 | N.D. | 6.2 | 76.1 | N.D. | N.D. | 4.6 | 2.9 | N.D. | N.D. | N.D. | N.D. | N.D. |

The purified peptide and hydrogel may be substantially endotoxin free without addition of a preservative or sterilization, as shown in Table 2. Thus, in some embodiments, the peptide preparation may be substantially free of a preservative.

TABLE 2
Endotoxin Levels of Different Compositions

| | OD at 410 nm | Endotoxin level (EU/mL) |
|---|---|---|
| Endotoxin free water | −0.000333333 | −0.016148109 |
| RO water | 3.436666667 | 1.267607913 |
| Tap water | 3.463666667 | 1.2776927 |
| Buffer solution | −0.056 | −0.036940201 |
| Peptide | 0.006 | −0.013782542 |
| Peptide hydrogel | 0.015 | −0.010420946 |

### The Self-Assembling Peptide Hydrogel

The preparations disclosed herein may be provided to self-assemble into a hydrogel having preselected properties. The polymeric hydrogel may have a substantially physiological pH. In general, the polymeric hydrogel may have a pH of between 4.0 and 9.0, for example, between 7.0 and 8.0, between 7.2 and 7.8, or between 7.3 and 7.5.

The polymeric hydrogel may be substantially transparent. For example, the polymeric hydrogel may be substantially free of turbidity, for example, visible turbidity. Visible turbidity may be determined by macroscopic and microscopic optical imaging. The polymeric hydrogel may be substantially free of peptide aggregates (peptide clusters), for example, visible peptide aggregates. Visible peptide aggregates may be determined by static light scattering (SLS) and UV-VIS testing. “Transparency” may refer to the hydrogel's ability to pass visible light. The substantially transparent hydrogel may have UV-VIS light absorbance of between about 0.1 to 3.0±1.5 at a wavelength of between about 205 nm to about 300 nm.

The assembled polymeric hydrogel may have a nano-porous structure. The polymeric hydrogel may be hydrated or substantially saturated. In some embodiments, the hydrogel may have between 90% w/v and 99.9% w/v aqueous solution, for example, between 92% w/v and 99.9% w/v or between 94% w/v and 99.9% w/v. The nano-porous structure may be selected to be impermeable to a target microorganism. Thus, the hydrogel may be used to encapsulate a target microorganism or to maintain the target site free from the target microorganism. The nano-porous structure may be selected to allow gaseous exchange at the target site. The polymeric hydrogel may have a nano-porous structure having a pore size of between 1 nm and 1000 nm, as selected (e.g., based on a target microorganism, target cell, or desired functionality). The polymeric hydrogel may have a fibril width of between 1 nm and 100 nm, as selected.

The hydrogel may generally be cationic in nature. In other embodiments, the hydrogel may be anionic in nature. In yet other embodiments, the hydrogel may be blended to contain multi-domains of cationic and/or anionic components. The hydrogel may be designed to have a preselected charge. The self-assembling peptide hydrogel disclosed herein may be tunable to biological functionality that supports the viability and function of transplanted therapeutic cells, to exhibit shear-thinning mechanical properties that allow easy and rapid administration in an intra-operative setting, to exhibit antimicrobial properties to control wound bioburden, to exhibit antiviral properties to treat or inhibit viral infection, and/or to exhibit antifungal properties to treat or inhibit fungal infection.

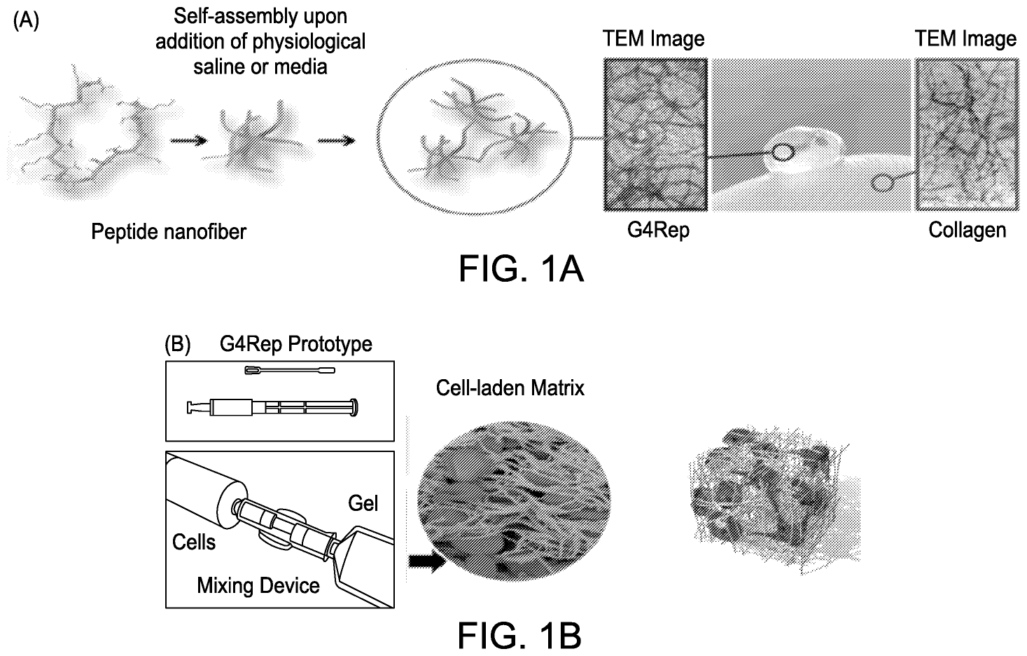

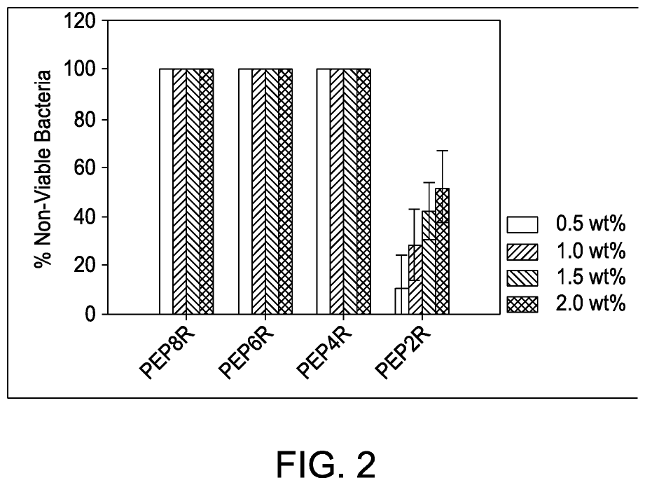

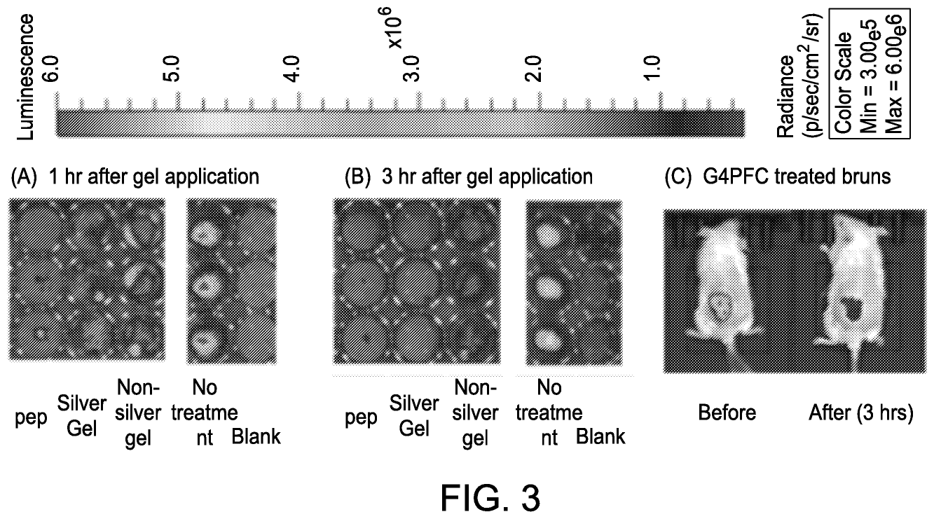

In particular, the peptide sequence and structure may include peptide functional groups that form nanofibers, which further self-assemble to form macromolecular structures (FIG. 1A-1B). The peptides may self-assemble in response to an environmental stimulus. The peptides may self-assemble in the presence of substantially physiological buffers, such as media or saline. The peptide hydrogels may assemble into an extracellular scaffolding matrix that is similar to native fibrillar collagen (FIG. 1A-1B). Schematics of gel matrix self-assembly and an exemplary nanostructure are shown in FIGS. 1A-1B. As shown in FIG. 1A, single peptide nanofibers self-assemble into a gel when ionic buffer is added. FIG. 1A includes a TEM image demonstrating that the nanostructure and pore size of the peptide gel look similar to native ECM (collagen). FIG. 1B includes a schematic drawing of an intra-operative mixing device for mixing a cell suspension with peptide gel matrix. A schematic SEM image in FIG. 1B of the cell-laden matrix demonstrates the exemplary concept of cells in matrix.

The peptide may be engineered by design to self-assemble into a hydrogel which is substantially biocompatible. The peptide may be engineered by design to self-assemble into a hydrogel that is cell friendly. In certain embodiments, the cell-friendly polymeric hydrogel may be non-inflammatory, and/or non-toxic. The cell-friendly polymeric hydrogel may be substantially biodegradable. The peptide may be engineered by design to be substantially antimicrobial, antiviral, and/or antifungal.

The short peptides and/or peptide functional groups may be produced synthetically. Thus, the peptides may provide ease of manufacturing, scale-up, and quality control. In general, the peptides may be manufactured without the use of plant or animal expression systems. The peptides may be substantially free of naturally occurring endotoxins and disease-transmitting pathogens. In addition, the peptide sequence and functional groups may be tuned, allowing a versatility in control and design of the assembled hydrogel, including with respect to physical and chemical properties, such as biodegradation, mechanical properties, and biological activity.

The peptide may have a functional group engineered for a target indication. For instance, the peptide may have a bioactive functional group. The target indication may be tissue engineering of a target tissue. The target indication may include, for example, cell culture, cell delivery, wound healing, and/or treatment of biofilm. Thus, the peptide may be engineered by design to self-assemble into a hydrogel which is substantially biocompatible. The peptide functional group may have between about 3 and about 30 amino acid residues. For example, the peptide functional group may have between about 3 and about 20 amino acid residues. The peptide functional group may have a sequence selected from RGD, IKVAV, YIGSR, LKKTETQ, SNKPGVL, PKPQQFFGLM, GKLTWQELYQLKYKGI, and GGG.

In some embodiments, the peptide may include a modification selected from a linker and a spacer. Peptide “linkers” may generally refer to short amino acid sequences included to link multiple domains of the peptide. Peptide “spacers” may generally refer to amino acid sequences positioned to link and control the spatial relationship of the multiple domains of the assembled protein. The linker or spacer may be hydrophobic or hydrophilic. The linker or spacer may be rigid or flexible. Exemplary spacers include aminohexanoic acid (Ahx) (hydrophobic) and poly (ethylene) glycol (PEG) (hydrophilic). Glycine rich spacers are generally flexible.

Exemplary bioactive functional groups include laminin, bone marrow homing, collagen (e.g., I, II, and VI), bone marrow purification, and RGD/fibronectin types. Exemplary bioactive functional groups include VEGF, Substance P, Thymosin Beta, Cardiac Homing Peptide, Bone Marrow Homing Peptide, Osteopontin, and Ostegenic peptide. Exemplary bioactive functional groups include those in Tables 3-5 below.

TABLE 3
Exemplary Bioactive Functional Groups

| ECM Motif | ECM Type |
|---|---|
| -GGPDSGR | Laminin |
| -GGSDPGYIGSR | Laminin |
| -GGSKPPGTSS | Bone marrow homing |
| -GGDGEA | Collagen II |
| -GGPFSSTKT | Bone marrow homing |
| -GGFLGFPT | Bone marrow purification |
| -GGRGDS | RGD/fibronectin |
| -GGIKVAV | Laminin |
| -GGFPDERGVEGPGP | Collagen I |
| -GGPRGDSGYRGDSF | Collagen VI |

TABLE 4
Exemplary Bioactive Functional Groups

| Protein/Peptide | Bioactive Motif |
|---|---|
| VEGF | GKLTWQELYQLKYKGI |
| Substance P | RPKPQQFFGLM |
| Thymosin Beta | SNKPG |
| Thymosin Beta | LKKTETQG |
| Cardiac Homing Peptide | CSTMLKAC |
| Bone Marrow Homing Peptide | GCPFSSTKTE |
| Osteopontin | TLEGTKKGHKLHLDY |
| Osteogenic Peptide | ALKRQGRTLYGFGG |

TABLE 5
Exemplary Bioactive Functional Groups

| Peptide name | Sequence | Source |
|---|---|---|
| RGD | XRGDX | Fibronectin, osteopontin, |
| | | BSP and others |
| pRGD/bsp-RGD | CGGNGEPRGDTYRAY | BSP |
| hBSP₂₇₈₋₂₉₃ | YESENGEPRGDNYRAY | BSP |
| PHSRN | PHSRN | Fibronectin |
| F105 | YKRSRYT | FGF-2 |
| F119 | KRTGQYKLGSKTGPGQK | FGF-2 |
| F36 | PDGRVD | FGF-2 |
| F77 | KEDGRLL | FGF-2 |
| GFOGER | GGYGGGPC(GPP)₅GFOGER | Collagen type I |
| | (GPP)₅GPC | |
| DGEA | DGEA | Collagen type I |
| P15 | GTPGPQGIAGQRGVV | Collagen type I |
| AC-100 | ERGDNDISPFSGDGQ | MEPE |
| ODP (osteopontin-derived | DVDVPDGRGDSLAYG | Osteopontin |
| peptide) | | |
| RRETAWA | RRETAWA | Identified by phage |
| | | display |
| YIGSR | YIGSR | Laminin |
| IKVAV | IKVAV | Laminin |
| Ln2-P3 | DLTIDDSYWYRI | Laminin |
| KRSR | KRSR | BSP, fibronectin, |
| | | osteopontin and others |
| Consensus heparin binding | FHRRIKA | BSP |
| domain | | |
| HBD peptide | RKKNPNCRRH | BMP-4 |
| BMP-2-derived peptide | NSVNSKIPKACCVPTELSAI | BMP-2 |
| BMP-2-derived peptide | KIPKASSVPTELSAISTLYL | BMP-2 |
| P24 | S^{[PO4]}KIPKASSVPTELSAIST | BMP-2 |
| | LYLDDD | |
| pBMP-9/SpBMP-9 | CGGKVGKA(CC/SS)VPTKL | BMP-9 |
| | SPISVLYK | |
| BMP-9-derived peptide | RKVGKASSVPTKLSPI SILYK | BMP-9 |
| BMP-7-derived peptide | RTVPKPSSAPTOLNAI STLYF | BMP-7 |
| BFP-1 | GQGFSYPYKA VESTQ | BMP-7 |
| OPD (osteopromotive-domain) | DWIVA | BMP-2 |
| CBM | GLRSKSKKFRRPDIQYPDA | Osteopontin |
| | TDEDITSHM | |
| BCSPTM_1 | NGLPGPIGP | Collagen type I |
| OGP (osteogenic growth | ALKRQGRTLYGFGG | Naturally occurring |
| peptide) | | |
| OGP₁₀₋₁₄ | YGFGG | OGP |
| PTH₁₋₃₄ | SVSEIQLMHNLGKHLNSME | Parathyroid hormone |
| | RVEWLRKKLQDVHNF | |
| CB | NGVFKYRRRYYLYKHAYF | BSP |
| | YPHLKRFPVQ | |
| CTC peptide | IAGVGGEKSGGF | Collagen-IIIa |
| Substance-P | RPKPQQFFGLM | Naturally occurring |
| Endothelin-1 | CSCSSLMDKECVYFCHLDIIW | Naturally occurring |
| QK | KLTWQELYQLKYKGI | VEGF |
| PBA2-1c | (CVRKIEIVRKK)₂-Ahx-Ahx- | PDGF |
| | Ahx-RKRKLERIAR | |
| Exendin 4 | HGEGTFTSDLSKQMEEEAVR | Glucagon-like peptide |
| | LFIEWLKNGGPSSGAPPPS | |
| OPD (osteopontin-derived | SVVYGLR | Osteopontin |
| peptide) | | |
| SPARC 113 | TLEGTKKGHKLHLDY | Osteonectin |
| SPARC 118 | KKGHK | Osteonectin |
| TP508 | AGYKPDEGKRGDACEGDS | Thrombin |
| | GGPFV | |
| RoY | YPHIDSLGHWRR | Designed |
| LL-37 | LLGDFFRKSKEKIGKEFKRI | hCAP18 |
| | VQRIKDFLRNLVPRTES | |

The peptide may have a functional group engineered to control or alter charge or pH of the peptide or preparation. A pre-selected charge or pH may provide bioactive properties. In some embodiments, a pre-selected charge or pH may provide antimicrobial, antifungal, and/or antiviral properties. In some embodiments, a pre-selected charge or pH may allow the preparation to be administered to a compatible target site.

The peptide may have an antimicrobial functional group. The antimicrobial functional group may include a conserved sequence of antimicrobial residues. In other embodiments, the antimicrobial functional group may overlap or partially overlap with the self-assembling functional group. In at least one embodiment, the peptide may have alternating or substantially alternating antimicrobial and self-assembling residues.

The peptide may have an antifungal functional group. The antifungal functional group may include a conserved sequence of antifungal residues. In other embodiments, the antifungal functional group may overlap or partially overlap with the self-assembling functional group. In at least one embodiment, the peptide may have alternating or substantially alternating antifungal and self-assembling residues.

The peptide may have an antiviral functional group. The antiviral functional group may include a conserved sequence of antiviral residues. In other embodiments, the antiviral functional group may overlap or partially overlap with the self-assembling functional group. In at least one embodiment, the peptide may have alternating or substantially alternating antiviral and self-assembling residues.

The self-assembled hydrogel may be designed to have cell protective properties at the target site. In particular, the self-assembled hydrogel may be designed to be protective against foreign microorganisms, e.g., pathogenic microorganisms. The self-assembled hydrogel may be designed to be protective against fungal organisms. The self-assembled hydrogel may be designed to be protective against immune attack from environmental immune cells. The antimicrobial, antiviral, antifungal, and/or protective properties of the hydrogel may not substantially affect the viability, growth, or function of cells at the target site.

The protective properties of the hydrogel may be engineered by altering the net charge of the peptides. In some embodiments, the net charge may be altered by controlling one or more of the presence or absence of cationic particles or peptides, the presence or absence of anionic particles or peptides, buffers, salts, peptide concentration, peptide purity, and the presence or absence of peptide counterions. The peptide may be engineered to have positively charged, negatively charged, hydrophobic, or hydrophilic amino acid residues. In an exemplary embodiment, the peptide may provide antimicrobial, antiviral, and/or antifungal properties by inclusion amino acids which are positively charged at a substantially neutral pH level. Such amino acids may include, for example, arginine, lysine, tryptophan, and histidine.

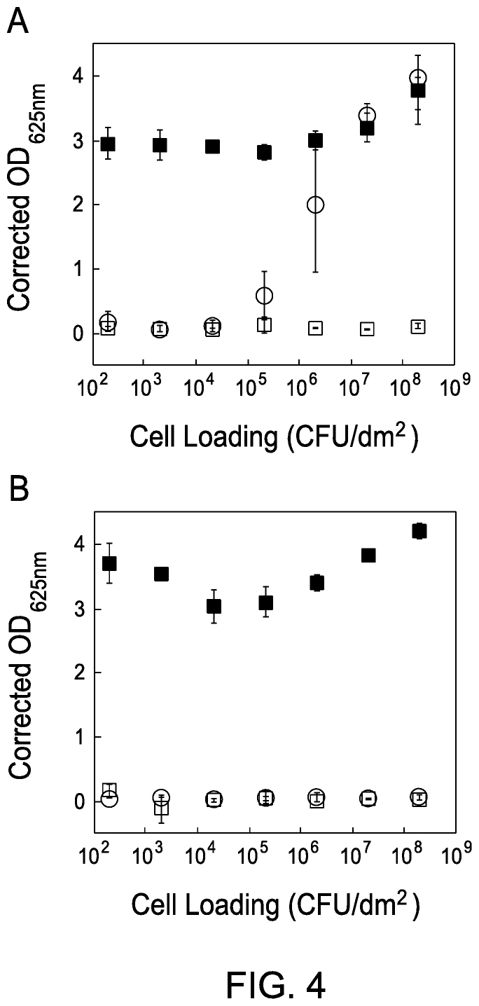

The peptide hydrogel may additionally exhibit antimicrobial properties. In general, the antimicrobial properties may be provided by including an antimicrobial functional group. In some embodiments, the antimicrobial functional group may include a cation-rich peptide sequence. In exemplary embodiments, the antimicrobial functional group may include varying ratios of lysine (K) and arginine (R) (FIG. 4). The antimicrobial peptide hydrogel may provide antimicrobial effects against gram-positive and negative bacteria, including, for example, E. coli (FIG. 4), S. aureus, and P. aeruginosa. FIG. 4 is a graph showing antimicrobial activity (as percent non-viable E. coli remaining after 24 hours of administration) of varying concentrations of peptides having 8 arginine residues (PEP8R), 6 arginine residues (PEP6R), 4 arginine residues (PEP4R), and 2 arginine residues (PEP2R).

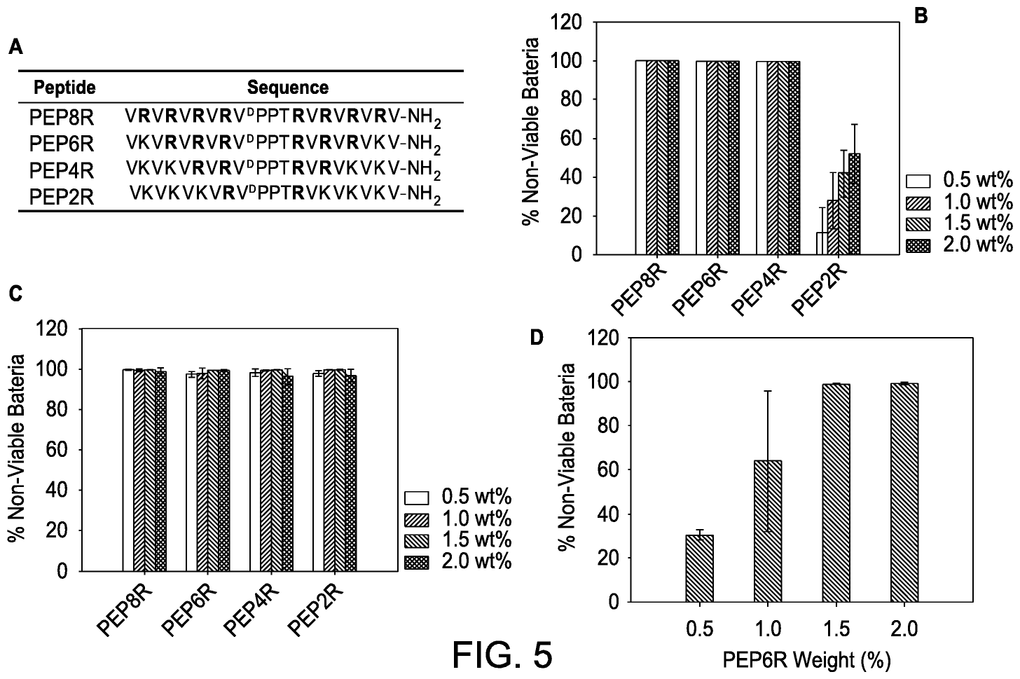

The peptide hydrogels may exhibit broad spectrum antimicrobial activity. In accordance with certain embodiments, the peptide hydrogels may reduce bioburden in vivo in partial thickness wounds inoculated with methicillin-resistant S. aureus (MRSA) (FIG. 5). FIG. 5 shows preliminary data demonstrating antimicrobial benefits of treating bioluminescent MRSA (US300) with peptide gels. Images (A) and (B) show wells plated with 100 μl of gel and 100 μl of US300 (1×10⁸ CFU/ml) demonstrating the antimicrobial activity of peptide gels compared to controls at 1 hour and 3 hours (n=3). Image (C) shows mice with partial thickness burns inoculated with 50 μl of 10⁸ CFU/ml US300 and treated with peptide gels. As shown in image (C), the mice exhibit reduced bioburden at 3 hours after administration.

In particular, the peptide hydrogel may exhibit antimicrobial properties against foreign and/or pathogenic microorganisms, and be compatible with the administered cells. For example, such peptide hydrogels may be compatible with mammalian erythrocytes and macrophages. In one exemplary experiment, when bacteria and mammalian cells were seeded simultaneously onto the peptide hydrogels disclosed herein, the bacteria were killed while the mammalian cells remained >90% viable after 24 hours and could continue to proliferate.

In some embodiments, the peptides may include functional groups to enhance or promote biological activity compatible or synergizing with MSC function. For example, in certain embodiments, the peptide sequence may contain a functional group that mimics fibronectin and promotes adhesion and proliferation of human MSCs to a greater extent than other ECM ligands. In certain embodiments, the peptide sequence may contain a functional group comprising a neuropeptide to promote diabetic wound healing by suppressing inflammation and inducing angiogenesis. In certain embodiments, the peptide sequence may contain a functional group comprising a neuropeptide to induce the proliferation and migration of MSCs, as well as enhance the immunomodulatory function of MSCs. In certain embodiments, the peptide sequence may contain a functional group to improve wound healing by increasing angiogenesis and inducing MSC proliferation and migration. In certain embodiments, the peptide sequence may lack a functional group that inhibits proteolytic activity. The peptide may be engineered to contain other functional groups known to one of skill in the art.

In vitro, the peptide hydrogels disclosed herein may allow cell invasion and proliferation in 3D constructs, allowing the hydrogels to serve as scaffolding matrices for tissue regeneration. The peptide hydrogels may show biocompatibility following subcutaneous implantation. Experiments show minimal cell debris or dead cells at the gel implantation site 7 days post-subcutaneous administration. Experiments further show minimal increases in cytokine concentration in the gel and surrounding tissues compared to naïve tissues, suggesting the gel has insignificant acute inflammation effects.

### Kits Comprising the Peptide Preparation

Kits comprising the peptide preparation are described herein. The kit may comprise the peptide preparation and a buffer solution. The buffer may be configured to induce self-assembly of the peptide prior to or concurrently with administration of the peptide. Each of the peptide preparation and the buffer may be included in a vial or chamber. For example, the kit may comprise a pre-filled packaging containing one or more of the preparation and the buffer. The kit may comprise one or more devices for use and/or delivery of the peptide preparation. The kit may comprise a mixing device. The kit may comprise a delivery device. In certain embodiments, the delivery device and/or mixing device may be the pre-filled packaging, for example, the kit may comprise a pre-filled syringe, spray bottle, ampule, or tube. FIG. 34 is a photograph of the preparation packaged in an end-use container. The exemplary end-use container of FIG. 34 is a pre-filled syringe. The end-use container may be employed as a delivery device or a mixing device. The kit and/or any component of the kit may be sterile or sterilized. For example, the kit and/or any component may be sterilized using autoclave sterilization, optionally terminal autoclave sterilization.

Any one or more component of the kit may be autoclavable. The packaged kit may be autoclavable. Any one or more component of the kit may be sterilized or sterile. For example, any one or more component of the kit may be sterilized by autoclave. The sterilized one or more component may be packaged in a substantially air-tight container. In some embodiments, the packaged kit may be sterilized, e.g., by autoclave.

In certain embodiments, the kit may comprise the purified peptide in a dried or powder form. For example, the purified peptide may be lyophilized. The kit may comprise a biocompatible solution to be combined with the purified peptide to produce the peptide preparation. In other embodiments, the kit may comprise instructions to combine the purified peptide with a biocompatible solution to produce the preparation. The kit may additionally comprise the buffer solution.

The kit may comprise instructions for use. In particular, the kit may comprise instructions to combine the buffer with the preparation, optionally in the mixing device, to form the hydrogel. A user may be instructed to combine the preparation and the buffer at the point of use. In some embodiments, the user may be instructed to combine the preparation and the buffer prior to administration or concurrently with administration. The user may be instructed to apply the preparation and the buffer to the target site separately.

The kit may additionally comprise instructions to store the kit under recommended storing conditions. For instance, the kit may comprise instructions to store the preparation or any component at room temperature (approximately 20-25° C.). The kit may comprise instructions to store the preparation or any component under refrigeration temperature (approximately 1-4° C.). The kit may comprise instructions to store the preparation or any component under freezer temperature (approximately 0 to −20° C.). The kit may comprise instructions to store the preparation or any component at body temperature (approximately 36-38° C.). The kit may comprise instructions to store the preparation or any component under cool and dry conditions.

The kit may additionally comprise an indication of expiration for the preparation or any component. The indication of expiration may be about 1 year after packaging. The indication of expiration may be between about 6 months and about 10 years after packaging, for example, between about 1 year and about 5 years after packaging.

The kit may comprise additional components for administration in combination with the preparation. In some embodiments, the kit may comprise instructions to combine the additional component prior to administration or concurrently with administration. The kit may comprise instructions to administer the preparation and the additional component to the target site separately. The additional component may be or comprise an antibacterial formulation, an antiviral formulation, an antifungal formulation, an anti-tumor formulation, an anti-inflammatory formulation, a cell culture media, a cell culture serum, an anti-odor formulation, an analgesic, a hemostat formulation, local anesthetic, or a pain-relief formulation. In particular embodiments, the kit may comprise a culture of cells for administration in combination with the preparation, as described herein. In some embodiments, the kit may further comprise a dressing, e.g., a topical dressing, a barrier dressing, and/or a wound dressing.

The kit may comprise one or more component configured to induce shear-thinning of the hydrogel. Mixing devices or delivery devices (described below) may be employed to induce shear-thinning of the hydrogel by mechanical agitation. The kit may comprise one or more component selected from a temperature control device, a pH control additive, an ion chelator composition, a solvent, a sound control device, a lyophilization device, and an air drying device to induce shear-thinning. For example, the kit may comprise a heater or cooler, a source of an acid or a base, a source of an ion chelator, a source of a solvent, a speaker or sound transmitter, a lyophilizer, or a compressed air dryer, or a fan.

### Mixing Devices

Mixing devices for preparation of a hydrogel at a point of care are disclosed herein. The device may be a multi-chamber device. In exemplary embodiments, the device may be a two-chamber device. The devices may include a first chamber for a peptide preparation. The preparation may comprise a self-assembling peptide in a biocompatible solution. The devices may include a second chamber for a buffer solution. The first chamber and the second chamber may be separated by a barrier provided to prevent fluid communication between the first chamber and the second chamber. The devices may, optionally, further comprise a mixing chamber. The mixing chamber may be fluidly connectable to the first chamber and the second chamber. Prior to mixing, the mixing chamber may be separated from the first chamber and/or the second chamber by a barrier. In other embodiments, the mixing device may be configured for direct mixing of the contents of the first and second chambers. In some embodiments, the devices may comprise a third chamber for an additional formulation or compound to be administered to the subject. The third chamber may be separated from the first chamber, the second chamber, and/or the mixing chamber. The third chamber may be fluidly connectable to the first chamber and/or the second chamber directly or via the mixing chamber.

The device may be a single use device. The device may be a multiple use device.

In an exemplary embodiment, each of the first, second, or third chamber may be a syringe barrel. Each barrel may have an associated plunger for agitation. Each barrel may be hermetically fitted to a coupling adapter, which forms the mixing chamber. The hermetic fitting may be, for example, a luer lock or luer taper connection.

The preparation and buffer may be agitated or otherwise mixed to form a homogenous or substantially homogenous mixture, inducing hydrogelation. In some embodiments, the preparation and buffer may be agitated by transferring the mixture back and forth between the first chamber and the second chamber. In some embodiments, the hydrogel exhibits shear-thinning properties, such that during agitation the mixture is substantially liquid. Upon settling, the mixture may form a solid or gel material.

In exemplary embodiments, the device may be configured to prepare a cell graft at a point of use. In use, the first chamber may comprise the cell preparation and the second chamber may comprise the peptide preparation. The cell preparation may comprise buffer. Alternatively, a third chamber may comprise buffer. Upon actuation the cell preparation and the peptide preparation may mix or contact, i.e. in the mixing chamber. The cells may be suspended in the peptide solution, forming a cell suspension comprising self-assembling peptides.

The cell preparation and the peptide preparation may be mixed with buffer, forming a buffer suspension. The buffer suspension may be agitated as described above, inducing self-assembly of the hydrogel. The buffer suspension may be agitated to disperse the cells, forming a homogenous or substantially homogenous mixture. The homogenous or substantially homogenous suspension may self-assemble to form a hydrogel cell graft.

The mixing device may be a static mixing device. A static mixer may generally comprise a device for substantially continuous mixing of the preparation without moving components. For example, the static mixer may comprise a cylindrical or rectangular housing with one or more inlet for each component to be mixed and a single outlet for the mixture. The static mixer may comprise a plate-type mixer.

The mixing device may generally be formed or lined with an inert, thermally-stable material. In certain embodiments, the material may be opaque and/or shatter resistant.

### Delivery Devices

In some embodiments, the kits may include a delivery device. For instance, the kits may include a syringe or catheter. The kits may include a dropper. The kits may include a spray, e.g. in conjunction with a bottle. The spray device may be, for example, a nasal spray. The kits may include a tube or ampule. The kits may include a film, for example. The type of delivery device may be selected based on a target indication. Additionally, the properties of the delivery device may be selected based on a target indication. For instance, a syringe for topical delivery of the preparation may have a larger passage than a syringe for injection of the preparation.

In some embodiments, the syringe may be used for topical application of the preparation. In other embodiments, the syringe may comprise a needle for parenteral application. The needle of the syringe may have a Birmingham system gauge between 7 and 34. The catheter may be used for parenteral application. The needle of the catheter may have a Birmingham system gauge between 14 and 26. The peptide may be formulated for administration through a particular gauge needle. For instance, the peptide may be selected to have a variable viscosity that will allow application of the preparation through a particular gauge needle.

In some embodiments, the spray bottle may be used for topical application of the preparation. The spray bottle may comprise a container for the preparation and a spray nozzle. The spray nozzle may be configured for targeted delivery to a target tissue. For instance, a spray nozzle for targeted delivery to an epithelial tissue may have a substantially flat surface and a spray nozzle for targeted delivery to an intranasal tissue may have a substantially conical surface. The spray nozzle may be configured to deliver a predetermined amount of the preparation. In some embodiments, the spray nozzle may be configured to deliver the preparation in substantially unidirectional flow, optionally, regardless of orientation of the spray bottle.

The spray nozzle may be configured to reduce retrograde flow. In certain embodiments, the spray nozzle may be spring-loaded. In other embodiments, the spray nozzle may be pressure actuated. The actuation pressure may be selected based on the variable viscosity of the preparation. For instance, the actuation pressure may be selected to be sufficient to dispense the hydrogel through the spray nozzle.

The film may be used for topical application of the preparation. The film may be saturated with the preparation. The film may be used as a barrier dressing and/or hemostat. In some embodiments, the film may accompany a barrier dressing.

The delivery device may be a single use device. The delivery device may be a multiple use device. The delivery device may include a first chamber for a peptide preparation. The preparation may comprise a self-assembling peptide in a biocompatible solution. The delivery device may include a second chamber for a buffer solution. The first chamber and the second chamber may be separated by a barrier provided to prevent fluid communication between the first chamber and the second chamber. The delivery device may be constructed and arranged for administration of the peptide preparation and the buffer solution simultaneously or substantially simultaneously. In some embodiments, the delivery device may comprise a third chamber for an additional formulation or compound to be administered to the subject. The third chamber may be separated from the first chamber and/or the second chamber.

The delivery device may generally be formed or lined with an inert, thermally-stable material. In certain embodiments, the material may be opaque and/or shatter resistant.

### Coated Medical or Surgical Devices

In some embodiments, medical or surgical tools may have at least a portion of an exterior surface coated with the preparations or hydrogels disclosed herein. The coating may enable the exterior surface of the tool to exhibit antimicrobial properties, reducing incidence of infection. The coating may enable the exterior surface of the tool to be biocompatible or cytocompatible, reducing rejection and adverse reaction from contact.

The surgical tool may be a surgical instrument. For example, the tool may be a grasper, such as forceps, clamp or occluder, needle driver or needle holder, a suture or suture needle, retractor, distractor, positioner, stereotactic device, mechanical cutter, such as scalpel, lancet, drill bit, rasp, trocar, ligasure, harmonic scalpel, surgical scissors, or rongeur, dilator, specula, suction tip or tube, sealing device, such as surgical stapler, irrigation or injection needle, tip and tube, powered device, such as drill, cranial drill, or dermatome, scopes or probe, including fiber optic endoscope and tactile probe, carrier or applier for optical, electronic, and mechanical devices, ultrasound tissue disruptor, cryotome, cutting laser guide, or a measurement device, such as ruler or caliper. Other surgical tools or instruments are within the scope of the disclosure.

The medical or surgical tool may be an implantable tool. For example, the medical or surgical tool may be an implantable device, such as, implantable cardioverter defibrillator (ICD), pacemaker, intra-uterine device (IUD), stent, e.g., coronary stent, ear tube, or eye lens. Other implantable tools are within the scope of the disclosure. The implantable medical or surgical tool may be a prosthetic or a portion of a prosthetic device, for example, a prosthetic hip, knee, shoulder, or bone or a portion of a prosthetic limb. The implantable medical or surgical tool may be a mechanical tool, such as a screw, rod, pin, plate, disk, or other mechanical support. The implantable medical or surgical tool may be a cosmetic tool, such as breast implant, calf implant, buttock implant, chin implant, cheekbone implant, or other plastic or reconstructive surgery implant. Other medical or implantable tools are within the scope of the disclosure.

The formulation and/or thickness of the coating may be selected to be temporary, for example, degrading within a pre-determined period of time, for example, less than about 3 months, less than about 1 month, or less than about 2 weeks. The formulation and/or thickness of the coating may be selected to be semi-permanent, for example, degrading within a predetermined period of time of between about 3 months and 3 years, or between about 6 months and 2 years. The formulation and/or thickness of the coating may be selected to be permanent, for example, having a lifespan of more than 2 years or more than 3 years, or having a lifespan longer than the predetermined period of time that the medical or surgical tool is in contact with the subject.

### Methods of Treatment by Administration of Peptide Hydrogels

In some embodiments, the preparations disclosed herein may be administered according to a predetermined regimen. The preparations disclosed herein may be administered daily, weekly, monthly, yearly, or bi-yearly.

The preparations disclosed herein may provide immediate release effects. For example, the onset of action of the active ingredient may be less than one minute, several minutes, or less than one hour.

The preparations disclosed herein may provide delayed release effects. For example, the onset of action of the active ingredient may be more than one hour, several hours, more than one day, more than several days, or more than one week.

The preparations disclosed herein may provide extended release effects. For example, the preparations may be effective for more than one day, more than several days, more than one week, more than one month, several months, or up to about 6 months.

The preparations disclosed herein may be administered in conjunction with a medical approach that treats the relevant disease or disorder or a symptom of the relevant disease or disorder. For example, the preparations may be administered in conjunction with a medical approach that is approved to treat the relative disease or disorder or a symptom of the relevant disease or disorder. The preparations may be administered in conjunction with a medical approach that is commonly used to treat the relevant disease or disorder or a symptom of the relevant disease or disorder.

The preparations disclosed herein may be administered in combination with a surgical treatment. The preparations disclosed herein may be administered to treat wounds associated with the surgical treatment and/or to prevent or treat biofilm.

The preparations disclosed herein may be administered in combination with an anti-inflammatory agent or treatment. Anti-inflammatory agent may refer to a composition or treatment which reduces or inhibits local or systemic inflammation. The anti-inflammatory agent may comprise, e.g., non-steroidal anti-inflammatory drugs (NSAID), antileukotrienes, immune selective anti-inflammatory derivatives (ImSAID), and/or hypothermia treatment.

The preparations disclosed herein may be administered in combination with an antibacterial, antiviral, and/or antifungal agent. Such agents may refer to compositions or treatments which eliminate or inhibit proliferation of bacterial, viral, and/or fungal organisms, respectively. Exemplary antibacterial agents include antibiotics and topical antiseptics and disinfectants. The antiviral agent may be a target-specific antiviral agent or a broad-spectrum antiviral agent (e.g., remdesivir, ritonavir/lopinavir). Exemplary local antiviral agents include topical antiseptics and disinfectants. Exemplary antifungal agents include antifungal antibiotics, synthetic agents (e.g., flucytosine, azoles, allylamines, and echinocandins), and topical antiseptics and disinfectants.

The preparations disclosed herein may be administered to treat a wound, for example, an acute, a sub-acute, or a chronic wound. The wound may be a surgical wound, laceration, burn wound, bite/sting wound, vascular wound (venous, arterial or mixed), diabetic wound, neuropathic wound, pressure wound, ischemic wound, moisture-associated dermatitis, or result from a pathological process. In certain embodiments, the preparations may be administered in an amount effective to treat diabetic foot ulcers (DFU). In certain embodiments, the preparations may be administered in an amount effective to treat gastrointestinal wounds, such as anal fistulas, diverticulitis, and ulcers. In particular, the preparations may be administered in an amount effective to promote infection free wound closures.

The preparations disclosed herein may be administered in combination with a treatment or agent to provide anesthesia and/or pain-relief, e.g., local anesthetic. “Anesthetic” may refer to a composition which provides temporary loss of sensation or awareness. The anesthetic may be a general anesthetic (e.g., GABA receptor agonists, NMDA receptor antagonists, or two-pore potassium channel activators) or a local anesthetic (e.g., ester group agents, amide group agents, and naturally derived agents).

The preparations may be administered in combination with an analgesic or pain-relief agent. “Analgesic” may refer to a composition for systemic treatment or inhibition of pain. The analgesic may comprise an anti-inflammatory agent or an opioid.

The preparations disclosed herein may be administered in combination with a hemostat agent. “Hemostat” may refer to a tool or composition to control bleeding. Exemplary hemostat compositions include collagen-based agents, cellulose-based agents, and chitosan-based agents.

The preparations disclosed herein may be administered in combination with a treatment or agent to enhance cell or tissue graft therapy. In certain embodiments, the preparations disclosed herein may be administered in combination with a treatment or agent to prevent or inhibit cell death and/or control or reduce inflammation. The preparations disclosed herein may be administered in combination with cell culture media or cell culture serum.

The administered peptide hydrogels may have an immediate local effect. For instance, the administered preparations may provide localized wound healing or injury treatment effects by closing the wound or filling a void. In certain embodiments, the administered hydrogels may have a systemic effect. For instance, the administered hydrogels may enable cell migration or communication between cell grafts and environmental cells, resulting in a systemic effect. In other embodiments, the administered hydrogels may enable delivery of cell products or byproducts, resulting in a systemic effect. The administered peptide hydrogels may have antimicrobial, antiviral, and/or antifungal properties at a localized site of administration. In other embodiments, the administered peptide hydrogels may provide systemic antimicrobial, antiviral, and/or antifungal properties.

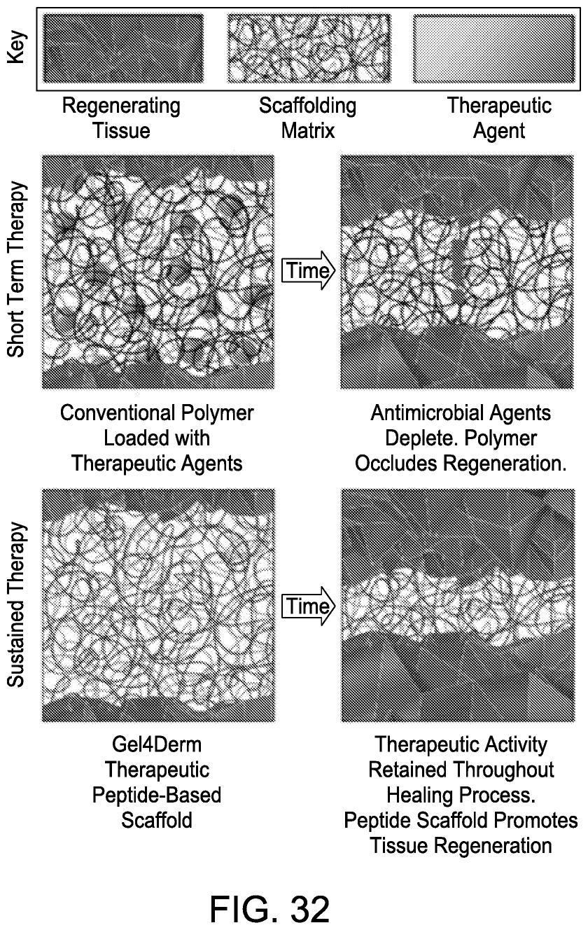

The administered peptide hydrogels may have long-term, sustained antimicrobial, antiviral, and/or antifungal properties at a localized site of administration. The peptide may be designed to form a hydrogel having a direct contact antimicrobial, antiviral, antifungal effect. Thus, the hydrogel may eradicate microorganisms which directly contact the hydrogel at the target site. The hydrogel may be substantially free of encapsulated antimicrobial, antiviral, and/or antifungal agents. Furthermore, the local antimicrobial, antiviral, and/or antifungal effect may persist as long as the hydrogel is in contact with the target tissue. FIG. 32 includes images which show sustained antimicrobial, antiviral, and/or antifungal effect at the target site.

To provide a systemic antimicrobial, antiviral, and/or antifungal effect, the peptide hydrogel may additionally comprise encapsulated antimicrobial, antiviral, and/or antifungal agents. Administration of such a hydrogel may generally provide: (1) local antimicrobial, antiviral, and/or antifungal treatment by direct contact as previously described, and (2) systemic antimicrobial, antiviral, and/or antifungal treatment as a vehicle of an encapsulated therapeutic agent.

The preparations disclosed herein may be formulated as a hemostat, debridement agent, or barrier dressing (e.g., antimicrobial, antifungal, or antiviral barrier dressing). The preparations may be formulated for wound treatment. Exemplary wounds which may be treated by administration of the preparation include partial and full thickness wounds (e.g., pressure sores, leg ulcers, diabetic ulcers), first and second degree burns, tunneled/undermined wounds, surgical wounds (e.g., associated with donor sites/grafts, tissue and cell grafts, Post-Moh's surgery, post laser surgery, podiatric, sound dehiscence), trauma wounds (e.g., abrasions, lacerations, burns, skin tears), gastrointestinal wounds (e.g., anal fistulas, diverticulitis, ulcers), and draining wounds. The preparations may be formulated for administration to a predetermined target tissue, for example, mesenchymal tissue, connective tissue, muscle tissue, nervous tissue, embryonic tissue, dermal tissue, bone tissue, dental tissue, corneal tissue, cutaneous tissue, integumental tissue, soft tissue, and hard tissue, or a biological fluid.

### Methods of Treatment of Microbial Infection

The preparation may be formulated to provide antimicrobial properties upon administration at a target site. For example, the self-assembled polymeric hydrogel may have antimicrobial properties. As disclosed herein, “antimicrobial” properties may refer to an effect against a microbial population, e.g., killing or inhibiting one or more microorganism from a microbial population. Thus, methods of treating a microbial infection or eliminating or inhibiting proliferation of a target microorganism are disclosed herein. “Proliferation” may generally refer to the metabolic or reproductive activity of an organism. Methods of reducing or eliminating a microbial contamination are disclosed herein. Methods of management of a microbial bioburden are disclosed herein.

For example, the methods may be employed for prevention or treatment of a microbial contamination. The microbial contamination may be identified by presence of one or more microorganism. In some embodiments, the methods may be employed for prevention or treatment of a microbial colonization or infection. The microbial colonization may refer to an established colony of one or more microorganism. The microbial infection may refer to an established colony of one or more microorganism which has been diagnosed by a clinical assessment. The microbial colonization or infection may be localized or systemic. In general, a microbial contamination may develop into a microbial colonization or infection if adequate treatment is not provided.

In some embodiments, the methods may be employed to treat and/or prevent chronic wounds, including, for example, diabetic wounds, e.g., diabetic foot ulcers (DFUs), leg ulcers, first and second degree burns, tunneled/undermined wounds, surgical wounds, e.g., donor sites/grafts, tissue and cell grafts, Post-Moh's surgery, post-laser surgery, podiatric, and sound dehiscence, trauma wounds, e.g., abrasions, lacerations, burns, and skin tears, gastrointestinal wounds (e.g., anal fistulas, diverticulitis, ulcers), and draining wounds. The methods may generally comprise administering the preparation in an amount effective to promote deactivation of a target microorganism associated with the wound.

In certain embodiments, the methods may be employed to treat and/or prevent surgical site infection. The methods may generally comprise administering the preparation in an amount effective to promote deactivation of a target microorganism.

In certain embodiments, antimicrobial effects may be obtained by administration of a cationic peptide. The cationic peptide may be configured to self-assemble into a polymeric form of a cationic peptide hydrogel. The net positive charge of the cationic peptide may be provided by including positive amino acid residues in the peptide structure, for example, in the folding group. The charge of the cationic peptide may be sufficient to provide the desired antimicrobial effect. For instance, in some embodiments the peptide may have a net charge up to +11. The peptide may have a net charge of +2 to +11, for example, +2 to +9 or +5 to +9. The cationic peptide and/or cationic hydrogel may promote deactivation of the target microorganism.

Hydrogel preparations of 3.0% w/v or less of the peptide may provide a suitable antimicrobial effect at the target site. However, it has been surprisingly discovered that hydrogel preparations comprising about 1.5% w/v or less or about 1.0% w/v or less of the peptide in the biocompatible solution may provide a suitable antimicrobial effect at the target site. For example, the preparation may comprise from about 0.1% w/v to about 1.5% w/v, about 0.25% w/v to about 1.5% w/v, about 0.5% w/v to about 1.0% w/v, about 0.5% w/v to about 0.8% w/v, about 0.7% w/v to about 0.8% w/v, or about 0.75% w/v to about 0.8% w/v of the peptide. In other embodiments, antimicrobial effects may be obtained by administering a preparation having more than 1.0% w/v of the peptide. For example, the preparation may comprise up to 8.0% w/v of the peptide.

The methods may comprise identifying a subject as being in need of treatment for a microbial contamination, colonization, or infection. The subject may be identified as having a localized contamination, colonization, or infection, or a systemic infection. The microbial contamination may be localized, for example, associated with a wound or contamination site, or systemic, for example, associated with more than one tissue and/or the bloodstream. The subject may be identified as being prone to having a microbial contamination, colonization, or infection. For example, the subject may have a wound that is at risk for developing a microbial infection. The subject may be of a population which is at risk for developing a microbial infection. For example, the subject may have an autoimmune disease, peripheral artery disease, or diabetes, or be undergoing chemotherapy.

In general, the microbial contamination, colonization, or infection may be induced by proliferation of a pathogenic microorganism (disease-causing microorganism). Thus, the methods may generally comprise administering the preparation in an amount effective to promote deactivation of a pathogenic microorganism. Administration may provide broad-spectrum antimicrobial treatment. In certain embodiments, administration may promote deactivation of multi-drug resistant organisms (MDRO). The pathogenic microorganism may be bacteria, archaea, fungi, algae, protozoa, and/or virus. The microbial contamination may be associated with a single pathogenic species or genus. The microbial contamination may be associated with a community of pathogens.

The pathogenic microorganism may be a bacterial microorganism. The preparation may provide a broad-spectrum antibacterial effect. For instance, administration may promote deactivation of gram-positive bacterial species. Administration may promote deactivation or gram-negative bacterial species. The pathogenic microorganism may be a species of a genus selected from Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yersinia.

The methods may comprise administering the preparation in an amount effective to sterilize at least 90% of the target microorganism at the target site. For instance, the methods may comprise administering the preparation in an amount effective to sterilize at least 90%, at least 95%, at least 97%, at least 98%, at least 99%, at least 99.9%, at least 99.99%, or at least 99.999% of the target microorganism at the target site or systemically. As disclosed herein, sterilize may refer to any process that eliminates, removes, kills, or deactivates the microorganism at the target site.

The preparation may be administered in combination with a surgical or other medical procedure. The procedure may be associated with debridement of a wound, burn, or infection, or a skin graft. In some embodiments, the method may comprise debridement of target tissue prior to administration of the preparation. The procedure may involve a surgical wound or incision. For example, the procedure may include the surgical removal of tissue or the repair of internal tissue. In other embodiments, the procedure may be minimally invasive. For example, the procedure may be a laparoscopy or robotic surgery.

In some embodiments, the preparation may be formulated as a hemostat. For example, the preparation may be administered as a hemostat and/or to control bleeding. The preparation may be administered directly to a bleeding wound. The preparation may be administered in response to bleeding.

In some embodiments, the preparation may be formulated as a debridement agent, e.g., autolytic debridement agent. For example, the preparation may be administered to a target tissue in an amount effective to debride or remove tissue. The preparation may be administered directly to a target tissue and/or wound for debridement. The preparation may be administered in response to an indication of dead or necrotic tissue. The preparation may be administered directly to dead or necrotic tissue.

In some embodiments, the preparation may be formulated as a barrier dressing. For instance, the preparation may be formulated as an antimicrobial barrier dressing. The formulation may be administered to protect underlying tissue from microbial contamination. For example, the formulation may be applied in an amount effective to protect tissue from microbial contamination. The preparation may be administered to defend the target tissue from recolonization by a microorganism. The preparation may be administered directly to the target tissue or wound to be protected. The preparation may be administered in response to a perceived risk of microbial contamination.

In some embodiments, the preparation may be formulated for wound treatment or management. For instance, the preparation may be administered to improve wound healing and tissue regeneration. In such embodiments, the preparation may be formulated as a tissue and cell substrate to improve wound healing and regeneration. Exemplary wounds which may be treated by administration of the preparation include partial and full thickness wounds (e.g., pressure sores, leg ulcers, diabetic ulcers), first and second degree burns, tunneled/undermined wounds, surgical wounds (e.g., associated with donor sites/grafts, tissue and cell grafts, Post-Moh's surgery, post laser surgery, podiatric, sound dehiscence), trauma wounds (e.g., abrasions, lacerations, burns, skin tears), gastrointestinal wounds (e.g., anal fistulas, diverticulitis, ulcers), and draining wounds.

Exemplary target sites include epithelial tissue, gastrointestinal system tissue, respiratory system tissue, cardiac system tissue, nervous system tissue, reproductive system tissue, ocular tissue, auditory tissue, and bloodstream. The target site may be a tissue selected from mesenchymal tissue, connective tissue, muscle tissue, nervous tissue, embryonic tissue, dermal tissue, bone tissue, dental tissue, corneal tissue, cutaneous tissue, integumental tissue, soft tissue, and hard tissue. Epithelial tissue may include, for example, epidermis, dermis, hair, and nail. The target site may be a biological fluid selected from tears, mucus, urine, menses, blood, wound exudates, and mixtures thereof. However, additional target sites may be treated by the methods disclosed herein.

The preparations and methods disclosed herein may be used for prophylactic care. For example, the preparations and methods may be used for prophylactic care in preventing infections, for example, wound infections, for example surgical site infections. The preparations and methods disclosed herein may be used for therapeutic care. For example, the preparations and methods may be used for therapeutic treatment in response to a diagnosed infection, for example, wound infection, for example, surgical site infection.

The methods may comprise administering a first dosage of the preparation. Treatment may cease after administration of the first dosage. For example, the first dosage may be a prophylactic dosage. In other embodiments, the first dosage may be a therapeutic dosage.

In some embodiments, the methods may comprise administering a booster dosage of the preparation at a time period subsequent to administration of the first dosage. The booster dosage may be administered on a regular pre-determined interval. The booster dosage may be administered as needed, for example, in response to an examination or diagnosis. Thus, the booster dosage may be a prophylactic dosage or a therapeutic dosage. More than one booster dosage of the preparation may be administered. The booster dosage may be administered approximately 1 hour, 2 hours, 6 hours, 12 hours, 18 hours, 24 hours, 2 days, 3 days, 5 days, 1 week, 2 weeks, 1 month, 2 months, 3 months, or 6 months after the first dosage.

The preparations may be administered by intra-tissue and/or as topical administration. The methods may comprise administering the preparation topically, enterally, or parenterally. For example, the methods may comprise administering the preparation topically to skin, mucous membranes, and/or tissues. The methods may comprise preparing the target tissue for topical application, for example, by cleaning the target tissue prior to administration. In certain embodiments, the methods may comprise surgically exposing the target tissue. The methods may comprise administering the preparation enterally with food and/or drink. The methods may comprise administering the preparation parenterally with assistance from a delivery device, for example, a needle or cannula. The preparations may be administered by other routes of administration.

The methods may comprise administering the preparation with the use of a delivery device. For example, the preparations may be administered by spray, dropper, film, squeeze tube, or syringe. The preparations may be administered surgically, for example, with a needle, catheter, endotracheal tube, cannula, or other invasive surgical device.

The preparations may be administered with at least one combination treatment selected from an antibacterial composition, an antifungal composition, an antiviral composition, an anti-tumor composition, an anti-inflammatory composition, a cell culture media, a cell culture serum, anti-odor composition, a hemostatic composition, and an analgesic or pain-relief composition. In accordance with certain embodiments, the preparations may be administered for treatment of infections with post-surgical debridement.

The preparations disclosed herein may be administered in combination with a medical approach that treats surgical site infection (SSI) or a symptom of SSI. For example, the preparations may be administered in combination with a medical approach that is approved to treat SSI or a symptom of SSI. The preparations may be administered in combination with a medical approach that is commonly used to treat SSI or a symptom of SSI.

The preparations disclosed herein may be administered in combination with a medical approach that treats wounds or scars. The medical approach may be one that is approved or commonly used to treat wounds or scars. Exemplary combination treatments may be ones approved or commonly used to treat at least one of partial and full thickness wounds (e.g., pressure sores, leg ulcers, diabetic ulcers), first and second degree burns, tunneled/undermined wounds, surgical wounds (e.g., associated with donor sites/grafts, tissue and cell grafts, Post-Moh's surgery, post laser surgery, podiatric, sound dehiscence), trauma wounds (e.g., abrasions, lacerations, burns, skin tears), gastrointestinal wounds (e.g., anal fistulas, diverticulitis, ulcers), and draining wounds.

In some embodiments, the preparations disclosed herein may be administered in combination with a medical approach that treats diabetic foot ulcer (DFU) or a symptom of DFU. For example, the preparations may be administered in combination with a medical approach that is approved to treat DFU or a symptom of DFU. The preparations may be administered in combination with a medical approach that is commonly used to treat DFU or a symptom of DFU.

In some embodiments, the preparations disclosed herein may be administered in combination with a medical approach that treats a gastrointestinal wound, such as anal fistulas, diverticulitis, ulcers, or a symptom of the gastrointestinal wound. The gastrointestinal wound may be associated with inflammatory bowel disease (IBD), for example, Crohn's disease or ulcerative colitis. For example, the preparations may be administered in combination with a medical approach that is approved to treat the gastrointestinal wound or IBD or a symptom the gastrointestinal wound or IBD. The preparation may be administered in combination with a medical approach that is commonly used to treat the gastrointestinal wound or IBD or a symptom of the gastrointestinal wound or IBD.

The following combination treatments are by way of example only. The preparation may be administered in combination with an aminosalicylate composition, a corticosteroid, an immunomodulator, a biologic therapy, such as an anti-tumor necrosis factor-alpha therapy, an anti-integrin therapy, or an anti-interleukin-12 or anti-interleukin-23 therapy, an antibiotic, an analgesic or pain reliever, an anti-diarrheal composition, an anti-nausea composition, or a vitamin. The preparation may be administered in combination with stem cells, Adalimumab, Infliximab, and/or anti-tumor necrosis factor alpha (TNF-α) agents. The preparation may be administered in combination with diet and nutrition guidelines.

The preparations disclosed herein may be administered in combination with an anti-inflammatory agent, e.g., non-steroidal anti-inflammatory drugs (NSAID), e.g., diclofenac, capsaicin, menthol, ibuprofen, felbinac, ketoprofen, piroxicam, aspirin, celecoxib, difunisal, etodolac, indomethacin, ketorolac, nabumetone, oxaprozin, salsalate, sulindac, or tolmetin, or corticosteroids, e.g., cortisol, cortisone, prednisone, prednisolone, methylprednisolone, dexamethasone, betamethasone, or triamcinolone. The anti-inflammatory agent may be administered topically, enterally, or parenterally. The preparations disclosed herein may be administered in combination with an anti-inflammatory treatment, e.g., hypothermia.

In certain embodiments, the preparations disclosed herein may be administered in combination with a treatment or agent to provide antimicrobial properties, and/or control or reduce inflammation. The preparations disclosed herein may be administered in combination with an antimicrobial agent, e.g., tetracycline, chloramphenicol, fluoroquinolones, cephalosporins, cephamycin, aminoglycosides, glycopeptides, or macrolides. The antimicrobial agent maybe administered topically, enterally, or parenterally.

The combination treatment and the preparation may be administered sequentially. The combination treatment may be administered prior to the preparation. In some embodiments, the method may comprise administering the preparation after a response to the combination treatment is detected. The combination treatment may be administered after the preparation. In some embodiments, the method may comprise administering the combination treatment after a response to the preparation is detected.

In some embodiments, the combination treatment may be administered substantially concurrently with the preparation. For example, the combination treatment may be combined with the preparation prior to administration. In some embodiments, the method may comprise administering the combination treatment and the preparation sequentially, prior to detecting a response by the first administration. For example, the combination treatment and preparation may be administered within 5 minutes of each other, within 4 minutes of each other, within 3 minutes of each other, within 2 minutes of each other, within 30 seconds of each other, or within 10 seconds of each other.

The method may comprise combining the peptide and the buffer prior to administration. For example, the method may comprise inducing gelation of the self-assembling peptide prior to administration. In some embodiments, the peptide and the buffer may be combined at the point of use. For instance, the peptide and the buffer may be combined at a point of care for the subject. The peptide and the buffer may be combined immediately prior to administration. The peptide and the buffer may be combined less than about 10 minutes prior to administration. The peptide and the buffer may be combined less than about 5 minutes prior to administration. For example, the peptide and the buffer may be combined less than about 4 minutes, less than about 3 minutes, less than about 2 minutes, less than about 1 minute, less than about 30 seconds, or less than about 10 seconds prior to administration.

In other embodiments, the peptide and the buffer may be combined during manufacture or distribution, or otherwise at a point upstream from the point of use. For instance, the preparation may be distributed or provided to a user as a hydrogel. Thus, the preparation may be stored as a hydrogel for a period of time prior to use.

In yet other embodiments, the buffer and the preparation may be combined upon administration. In some embodiments, the preparation and the buffer may be administered sequentially. The buffer may be administered prior to the preparation. The buffer may be administered after the preparation.

In some embodiments, the buffer may be administered substantially concurrently with the preparation. For example, the buffer and preparation may be administered within 5 minutes of each other, within 4 minutes of each other, within 3 minutes of each other, within 2 minutes of each other, within 30 seconds of each other, or within 10 seconds of each other.

### Methods of Eliminating or Inhibiting Proliferation of Microorganisms

The methods disclosed herein may be employed to eliminate microorganisms. Elimination of microorganisms may comprise, for instance, sterilization and or killing microorganisms or a target percentage of microorganisms. The methods may comprise eliminating, e.g. sterilizing or killing, at least 80%, 90%, 95%, 99%, 99.9%, 99.99%, or 99.999% of a target microorganism population at a target site. In at least some embodiments, the methods may comprise eliminating, e.g., sterilizing or killing, 100% of a target microorganism population at a target site. For example, the methods may comprise eliminating, e.g., sterilizing or killing, the target percentage of a community of microorganisms having up to 10⁸ CFU/ml. In some embodiments, the community of microorganisms may have between 10⁴-10⁸ CFU/ml or between 10⁵-10⁷ CFU/ml.

The methods disclosed herein may be employed to inhibit proliferation of microorganisms. Inhibiting proliferation of microorganisms may comprise, for example, preventing the spread or increase of microbial population at the target site.

Any one or more of the methods described above with respect to treatment of microbial infections may be employed to eliminate or inhibit proliferation of microorganisms at a target tissue site.

The methods disclosed herein may be employed to eliminate or inhibit proliferation of microorganisms, e.g., target microorganisms, on a surface. For instance, the preparations may be administered or applied to a target surface to eliminate or inhibit proliferation of microorganisms thereon. In accordance with certain embodiments, the target surface may be a household surface, an industrial surface, a food industry surface, or a healthcare surface. In some embodiments, the surface may be a medical surface, such as a medical tool surface, a medical implant surface, or a medical device surface. The medical tool may be a surgical or examination tool. The medical implant may be a prosthetic device, for example, an implantable or topical prosthetic device, or a structural support device, for example, a stent, valve, suture, mesh, pin, rod, screw, or plate. The medical device may be a monitoring or therapeutic medical device. The medical device may be an implantable or topical device. The medical surface may be implantable, partially implantable, or topical.

Thus, the preparations disclosed herein may be administered to treat or cleanse a surface. In some embodiments, treating a surface may comprise administering the preparations disclosed herein to proactively prevent a surface from developing a microbial population. In some embodiments, cleansing a surface may comprise administering the preparations disclosed herein to eliminate or inhibit proliferation of an existing microbial population. The methods may comprise identifying a surface as being in need of treatment or cleansing. For example, the methods may comprise testing a surface for the presence or absence of a target microorganism population and, optionally, treating or cleansing the surface responsive to a positive test result.

### Methods of Treatment of Fungal Infection

The preparation may be formulated to provide antifungal properties upon administration at a target site. For example, the self-assembled polymeric hydrogel may have antifungal properties. As disclosed herein, “antifungal” properties may refer to an effect against a fungal population, e.g., killing or inhibiting one or more organism from a fungal population. Thus, methods of treating a fungal infection or inhibiting proliferation of a fungal organism are disclosed herein. The methods may generally comprise administering the preparation in an amount effective to promote deactivation of a fungal organism. Methods of reducing or eliminating a fungal contamination are disclosed herein. In exemplary embodiments, a preparation comprising about 3.0% w/v or less of the peptide, for example, 1.5% w/v or less, or 1.0% w/v or less, may provide antifungal properties at a target site.

The methods may comprise identifying a subject as being in need of treatment for a fungal contamination, colonization, or infection. In certain embodiments, the preparation may be administered in an amount effective to treat at least one of biofilm, Tinea corporis, Candidiasis, Blastomycosis, Coccidioidomycosis, Histoplasmosis, Cryptococcosis, Paracoccidioidomycosis, Aspergillosis, Aspergilloma, Meningitis, Mucormycosis, Pneumocystis pneumonia (PCP), Talaromycosis, Sporotrichosis, and Eumycetoma of the subject. In some embodiment, the fungal organism may be a species of a genus selected from Aspergillus and Candida.

The preparations and methods may be effective at promoting deactivation of broad-spectrum (sporulating and non-sporulating) fungal organisms. The preparation may be administered in an amount effective to treat a fungal infection associated with or inhibit proliferation of at least one of Aspergillus clavatus, Aspergillus fischerianus, Aspergillus flavus, Aspergillus fumigatus, Aspergillus niger, Trichophyton mentagrophytes, Trichophyton rubrum, Microsporum canis, Candida albicans, Candida auris, Candida parapsilosis, Candida tropicalis, Blastomyces dermatitidis, Coccidioides immitis, Coccidioides posadasii, Cryptococcus gattii, Cryptococcus neoformans, Histoplasma capsulatum, Paracoccidioides brasiliensis, Pneumocystis jirovecii, Mucormycetes rhizopus, Mucormycetes mucor, Mucormycetes lichtheimia, Talaromyces marneffei, Sporothrix schenckii, Acremonium strictum, Noetestudina rosatii, Phaeoacremonium krajdenii, Pseudallescheria boydii, Curvularia lunata, Cladophilaophora bantiana, Exophiala jeanselmei, Leptosphaeria senegalensis, Leptosphaeria tompkinsii, Madurella grisea, Madurella mycetomatis, Pyrenochaeta romeroi, Trichosporon asahii, Cladosporium herbarum, and Fusarium sporotrichioides.

The preparation may be administered in combination with a surgical procedure. The methods may comprise administering the preparation in an amount effective to sterilize at least 90% of the fungal organism at the target site. For instance, the methods may comprise administering the preparation in an amount effective to sterilize at least 90%, at least 95%, at least 97%, at least 98%, at least 99%, at least 99.9%, at least 99.99%, or at least 99.999% of the fungal organism at the target site. Exemplary target sites may include epithelial tissue, oral tissue, esophageal tissue, tracheal tissue, pulmonary tissue, cardiac tissue, kidney tissue, ocular tissue, and bloodstream. Epithelial tissue may include, for example, epidermis, dermis, hair, and nail. However, additional target sites may be treated by the methods disclosed herein. As disclosed herein, sterilize may refer to any process that eliminates, removes, kills, or deactivates the fungal organism at the target site.

### Methods of Treatment of Biofilm

The preparation may be formulated for treatment of biofilm. Thus, the methods disclosed herein may comprise treatment of biofilm. Treatment of biofilm may generally comprise eliminating at least a portion of biofilm or inhibiting biofilm formation. Administration of the preparation may have an effect against a biofilm population, for example, killing or inhibiting one or more organism in a biofilm community. In general, the charged peptide polymer hydrogel may deconstruct the polymicrobial fungal and bacterial biofilm barrier upon contact. While not wishing to be bound by theory, it is believed the preparations disclosed herein may be selected to dismantle extracellular matrix of the biofilm population, exposing fungal, viral, and microbial organisms of the biofilm to the cationic peptide of the hydrogel. The peptide hydrogel may be effective by destroying microbes, fungi, and viral organisms within biofilms. The preparation may be administered as an antifungal, antimicrobial, and/or antiviral peptide to destroy fungi, microorganisms, and/or viral organisms, e.g., in a biofilm population, through cell lysis.

Methods of management of biofilm are also disclosed herein. For example, the methods may be employed for prevention of biofilm. The preparation may be administered to a target tissue having a population of biofilm or identified as prone to developing biofilm, e.g., a wound or wounded tissue. The preparation may be administered in response to tissue contamination or odor.

The methods may generally comprise administering the preparation in an amount effective to promote treatment of biofilm and/or deactivation of a biofilm population. The biofilm population may comprise bacterial organisms, for example, gram-positive and/or gram-negative bacterial organisms. The biofilm population may comprise fungal organisms, for example, sporulating and/or non-sporulating fungal organisms. Thus, the preparation may provide treatment of biofilm by the antimicrobial and/or antifungal properties and methods described above. In certain embodiments, the biofilm population may comprise viral organisms. The preparation may provide treatment of biofilm by antiviral properties and methods described herein.

The preparation may be formulated as a biofilm removal agent. In some embodiments, the preparation may be administered to a target tissue for removal of biofilm. For example, the preparation may be administered for debridement of the biofilm and/or biofilm-infected tissue.

### Methods of Treatment of Viral Infection

The preparation may be formulated to provide antiviral properties upon administration at a target site. For example, the self-assembled polymeric hydrogel may have antiviral properties. As disclosed herein, “antiviral” properties may refer to an effect against a viral population, e.g., killing or inhibiting one or more organism from a viral population. Thus, methods of treating a viral infection or inhibiting proliferation of a viral organism are disclosed herein. The methods may generally comprise administering the preparation in an amount effective to promote deactivation of a viral organism. Methods of reducing or eliminating a viral contamination are disclosed herein. In exemplary embodiments, a preparation comprising about 3.0% w/v or less of the peptide, for example, 1.5% w/v or less, or 1.0% w/v or less, may provide antiviral properties at a target site.

The methods may comprise identifying a subject as being in need of treatment for a viral contamination, colonization, or infection. In certain embodiments, the preparation may be administered in an amount effective to treat at least one of a respiratory viral colonization or infection (e.g., associated with rhinovirus, influenza, coronavirus, or respiratory syncytial virus), a viral skin infection (e.g., associated with molluscum contagiosum, herpes simplex virus, or varicella-zoster virus), a foodborne viral infection (e.g., associated with hepatitis A, norovirus, or rotavirus), a sexually transmitted viral infection (e.g., associated with human papilloma virus, hepatitis B, genital herpes, or human immunodeficiency virus), and other viral infections (e.g., associated with Epstein-Barr virus, West Nile virus, or viral meningitis) of the subject.

The preparation may be administered in combination with a surgical procedure. The methods may comprise administering the preparation in an amount effective to sterilize at least 90% of the viral organism at the target site. For instance, the methods may comprise administering the preparation in an amount effective to sterilize at least 90%, at least 95%, at least 97%, at least 98%, at least 99%, at least 99.9%, at least 99.99%, or at least 99.999% of the viral organism at the target site or systemically. In some embodiments, the methods may comprise administering the preparation in an amount effective to sterilize 100% of the viral organism at the target site or systemically. In certain embodiments, the preparation may be administered in an amount effective to treat biofilm or kill or deactivate a biofilm population containing a viral organism.

Exemplary target sites may include epithelial tissue, oral tissue, esophageal tissue, tracheal tissue, pulmonary tissue, cardiac tissue, kidney tissue, ocular tissue, and bloodstream. However, additional target sites may be treated by the methods disclosed herein. As disclosed herein, sterilize may refer to any process that eliminates, removes, kills, or deactivates the viral organism at the target site.

### Methods of Administration of Peptide Hydrogels

The peptide hydrogels may be administered by any mode of administration known to one of skill in the art. The method of administration may comprise selecting a target site of a subject and administering the preparation to the target site. In certain embodiments, the methods may comprise mixing the peptide with a buffer configured to induce self-assembly of the peptide to form the hydrogel. In general, the peptide may be mixed with the buffer prior to administration. However, in some embodiments, the peptide may be combined with the buffer at the target site.

The target site may be any bodily tissue or bloodstream. In some embodiments, the target site may be epithelial tissue, gastrointestinal system tissue, respiratory system tissue, cardiac system tissue, nervous system tissue, reproductive system tissue, ocular tissue, or auditory tissue. The route of administration may be selected based on the target tissue. Exemplary routes of administration are discussed in more detail below.

In some embodiments, the methods may comprise identifying a subject in need of administration of the preparation. The methods may comprise imaging a target site or monitoring at least one parameter of the subject, systemically or at the target site. Exemplary parameters which may be monitored include temperature, pH, response to optical stimulation, and response to dielectric stimulation. Thus, in some embodiments, the method may comprise providing optical stimulation or dielectric stimulation to the subject, optionally at the target site, for measuring a response. The response may be recorded, optionally in a memory storing device. In general, any parameter which may inform a user of a need or desire for administration of the preparation may be monitored and/or recorded. The methods may comprise imaging the target site or monitoring at least one parameter of the subject prior to administration of the preparation, concurrently with administration of the preparation, or subsequent to administration of the preparation. For example, the methods may comprise imaging the target site or monitoring at least one parameter of the subject after an initial dose and before a potential subsequent dose of the preparation.

In certain embodiments, the preparation may be administered responsive to the measured parameter being outside tolerance of a target value. The preparation may be administered automatically or manually in response to the measured parameter.

The preparation may be formulated for topical, parenteral, or enteral administration. The preparation may be formulated for systemic administration. Various pharmaceutically acceptable carriers and their formulations are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2 S, 1988; Aulton, M. and Taylor, K., Aulton's Pharmaceutics: The Design and Manufacture of Medicines, 5th Edition, 2017; Antoine, A., Gupta M. R., and Stagner, W. C., Integrated Pharmaceutics: Applied Preformulation, Product Design, and Regulatory Science, 2013; Dodou K. Exploring the Unconventional Routes—Rectal and Vaginal Dosage Formulations, The Pharmaceutical Journal, 29 Aug. 2012.

### Parenteral Administration of Peptide Hydrogels

In certain embodiments, the hydrogels may be administered parenterally. In general, parenteral administration may include any route of administration that is not enteral. The preparation may be administered parenterally via a minimally invasive procedure. In particular embodiments, the parenteral administration may include delivery by syringe, e.g., by needle, or catheter. For instance, the parenteral administration may include delivery by injection. The parenteral administration may be intramuscular, subcutaneous, intravenous, or intradermal. The shear-thinning ability of the hydrogels may allow distribution through small lumens, while still providing minimally invasive treatment.

The method may comprise applying mechanical force to the hydrogel for parenteral administration. The hydrogel may be thinned by applied mechanical force, for example, pressure applied by a syringe to administer the preparation by injection. In particular, the pressure applied to administer the preparation through a needle or catheter may be sufficient to shear thin the hydrogel for application.

The peptide hydrogels may be administered parenterally to any internal target site in need thereof. For instance, cardiac tissue, nervous tissue, connective tissue, epithelial tissue, or muscular tissue, and others, may be the target site. The peptide hydrogels may be administered parenterally to a target site of a solid tumor. In exemplary embodiments, antifungal treatment of pulmonary tissue may be provided by parenteral administration of the peptide hydrogels described herein.

### Topical Administration of Peptide Hydrogels

In certain embodiments, the hydrogels may be administered topically. In general, topical administration may include any external or transdermal administration. For instance, the target site for administration may be an epithelial tissue. In particular embodiments, the topical administration may be accompanied by a wound dressing or hemostat.

The preparation may be administered topically with a delivery device. For instance, the preparation may be administered topically by spray, aerosol, dropper, tube, ampule, film, or syringe. In particular embodiments, the preparation may be administered topically by spray. The spray may be, for example, a nasal spray. Spray parameters which may be selected for administration include droplet size, spray pattern, capacity, spray impact, spray angle, and spray diameter. Thus, the methods may comprise selecting a spray parameter to correlate with the target site or target indication. For instance, a smaller spray diameter may be selected for administration to a small wound. A specific spray angle may be selected for administration to a target site which is difficult to reach. A denser spray pattern or larger droplet size may be selected for administration to a moist target site.

Exemplary droplet sizes may be between 65 μm to 650 μm. For instance, fine droplets having an average diameter of 65 μm to 225 μm, medium droplets having an average diameter of 225 μm to 400 μm, or coarse droplets having an average diameter of 400 μm to 650 μm may be selected. The spray pattern may range from densely packed droplets to sparse droplets. The spray diameter may range from less than 1 cm to 100 cm. For instance, spray diameter may be selected to be between less than 1 cm and 10 cm, between 10 cm and 50 cm, or between 50 cm and 100 cm. Spray angle may range from 0° to 90°. For instance, spray angle may be selected to be between 0° and 10°, between 10° and 45°, or between 45° and 90°.

In some embodiments, the preparation may be administered topically with a film. The film may be a rigid, semi-flexible, or flexible film. In certain embodiments, the flexible or semi-flexible film may be configured to adopt a topological conformation of the target site. In general, the film may be in the form of a substrate saturated with the preparation or hydrogel. The substrate may be rigid, semi-flexible, or flexible. The film may be administered as a barrier dressing and/or hemostat. The preparation may be administered topically with a film and accompanied by a barrier dressing. The peptide formulated as a saturated film or barrier dressing may provide antimicrobial, antiviral, and/or antifungal treatment by direct contact with target population, as previously described. Conventional antimicrobial wound dressings rely on traditional antibiotics and function merely as a vehicle for antibiotic agents. However, the peptide hydrogel saturated film or barrier dressing described herein may be designed to provide a biophysical mode of cell-membrane disruption against broad-spectrum (gram-positive and gram-negative) bacterial cultures. Thus, the antimicrobial, antiviral, and/or antifungal peptide hydrogel saturated film or barrier dressing may generally avoid concerns around minimum inhibitory bacterial concentrations typical to conventional small molecule loaded polymers. Instead, the peptide hydrogel disclosed herein may be designed to exert toxicity against gram-positive and gram-negative bacteria (including antibiotic resistant strains) while remaining cell-friendly, non-inflammatory, and non-toxic by selecting amino acid charge ratio of the peptide. Similarly, the peptide hydrogel disclosed herein may be designed to exert toxicity against fungal organisms (e.g., sporulating and non-sporulating fungal organisms) and/or viral organisms. The saturated film or barrier dressing disclosed herein may provide a temporary extracellular matrix scaffold for tissue regeneration.

The peptide hydrogels may be administered topically to any target site in need thereof. Wound healing, e.g., diabetic wound healing, is described herein as one exemplary embodiment. However, it should be understood that many other topical target sites and treatments are envisioned, for example, as previously described above. The wounds may include acute, sub-acute, and chronic wounds. The wound may be a surgical wound or ischemic wound. Chronic wounds such as venous and arterial ulcer wounds or pressure ulcer wounds, and acute wounds, such as those caused by trauma may be treated. In some embodiments, the preparation may be formulated as a film, barrier dressing, and/or hemostat. Administration of the preparation may accompany a barrier dressing and/or hemostat.

Treatment and/or management or inhibition of biofilm is described herein as another exemplary embodiment. Moisture management and/or exudate management of wounds or tissues is described herein as another exemplary embodiment. Tissue debridement is described herein as another exemplary embodiment. The preparation may be administered topically as a prophylactic, for example, in association with a wound. The preparation may be administered topically as an analgesic, for example, to a chronic wound or site of biofilm.

### Enteral Administration of Peptide Hydrogels

In certain embodiments, the hydrogels may be administered enterally. In general, enteral administration may include any oral or gastrointestinal administration. For instance, the target site for administration may be an oral tissue or a gastrointestinal tissue. In particular embodiments, the enteral administration may be accompanied by food or drink. The preparation may be administered on a substantially empty stomach. In some embodiments, water is administered to the subject after administration of the preparation. In some embodiments, several hours are waited prior to food consumption after administration.

Such enteral preparations may be formulated for oral, sublingual, sublabial, buccal, or rectal application. Oral application formulations are generally prepared specifically for ingestion through the mouth. Sublingual and sublabial formulations, e.g., tablets, strips, drops, sprays, aerosols, mists, lozenges, and effervescent tablets, may be administered orally for diffusion through the connective tissues under the tongue or lip. Specifically, formulations for sublingual administration may be placed under the tongue and formulations for sublabial administration may be placed between the lip and gingiva (gum). Sublabial administration may be beneficial when the dosage form comprises materials that may be corrosive to the sensitive tissues under the tongue. Buccal formulations may generally be topically held or applied in the buccal area to diffuse through oral mucosa tissues that line the cheek. Rectal application may be achieved by inserting the formulation in the rectal cavity, either with or without the assistance of a device. Device-assisted application may include, for example, delivery via an applicator or an insertable applicator, catheter, feeding tube, or delivery in conjunction with an endoscope or ultrasound. Suitable applicators include liquid formulation bulbs and launchers and solid formulation insertable applicators.

For any of the routes of administration disclosed herein, the methods may comprise administering a single dosage of the preparation. The site of administration may be monitored for a period of time to determine whether a booster or subsequent dosage of the preparation is to be administered. For example, the methods may comprise monitoring the site of administration. A parameter of the subject, optionally at the target site, may be monitored as previously described. The subject may be monitored hourly, every 2-3 hours, every 6-8 hours, every 10-12 hours, every 12-18 hours, or once daily. The subject may be monitored daily, every other day, once every few days, or weekly. The subject may be monitored monthly or bi-monthly. In certain embodiments, the subject may be monitored for a period of up to about 6 months. For example, the subject may be monitored for about 1 month, about 2 months, about 3 months, about 4 months, about 5 months, or about 6 months.

The methods may comprise administering at least one booster or subsequent dosage of the preparation. For example, the methods may comprise administering a booster dosage to the target site at least about 1 day, 2 days, 3 days, 4 days, 5 days, 6 days, 7 days, 8 days, 9 days, 10 days, 11 days, 12 days, 13 days, or 14 days after the first dosage. The methods may comprise administering a booster dosage 1 week, 2 weeks, 3 weeks, 4 weeks, 5 weeks, or 6 weeks after the first dosage. The methods may comprise administering a booster dosage at least 6 months or 1 year after the first dosage. In certain embodiments, at least a portion of the hydrogel may be present at the target site at the time of the booster dosage. In other embodiments, the hydrogel may be fully metabolized or otherwise eliminated from the target site at the time of the booster dosage.

Methods of Biological Material Delivery with Peptide Hydrogels

Methods of administering biological material to a subject are disclosed herein. The methods may generally include suspending the biological material in a hydrogel. The biological material may be combined with a preparation comprising a self-assembling peptide in a biocompatible solution and a buffer comprising an effective amount of an ionic salt and a biological buffering agent to induce self-assembly of the hydrogel. The methods may comprise administering an effective amount of the biological material, the preparation, and the buffer (optionally in hydrogel form) to a target site of the subject. Suspending the biological material with the preparation or the buffer will generally produce a liquid suspension. Combining the preparation with the buffer may trigger gelation of the suspension into a hydrogel comprising the biological material.

The biological material to be administered may include biological fluids, cells, and/or tissue material. In some embodiments, one or more biological material administered may be synthetic. For instance, the biological fluids may be or include synthetic fluids. In other embodiments, the biological material may be obtained from a donor. The biological material may be autologous (obtained from the recipient subject). The biological material may be allogeneic (obtained from a donor subject of the same species as the recipient subject) or xenogeneic (obtained from a donor subject of a different species as the recipient subject).

The self-assembled hydrogel may have a physical structure substantially similar to the native extracellular matrix of the biological material, allowing the gel to serve as a temporary scaffold to promote cell growth, function, and/or viability. In particular, the self-assembled hydrogel may have a similar properties, including, for example, pore size, density, hydration, charge, rigidity, etc., to the native extracellular matrix of the biological material. The properties may be selected responsive to the biological material type.

The self-assembled hydrogel may have a selected degradation rate. The degradation rate may be selected responsive to the target site of implantation or administration. The self-assembled hydrogel properties may be selected to promote migration of cells to the hydrogel environment. The self-assembled hydrogel properties may be selected to promote cell protection in a hostile environment. The self-assembled hydrogel properties may be selected to promote anchoring of the biological material within the hydrogel, for example, as with cells that will not engraft onto host tissue. The self-assembled hydrogel properties may be selected to promote migration of cell products or byproducts or tissue-derived material to the hydrogel environment, for example, growth factors, exosomes, cell lysates, cell fragments, or genetic material. In an exemplary embodiment, the self-assembled hydrogel properties may be selected to control differentiation of cells, e.g., progenitor cells or stem cells, e.g., mesenchymal stem cells.

The properties of the self-assembled hydrogel may be controlled by designing the peptide. For example, the peptide may include functional groups that provide one or more selected physical property. The properties may be controlled by selecting the composition of media or buffer. For example, the media may include serum or be substantially free of serum. For example, the buffer may have a net positive charge, be net neutral, or have a net negative charge. In some embodiments, the functional group may be configured to alter peptide net charge or counterions when the peptide is suspended in the solution.

The administration of cells and cell products, byproducts, tissue, and tissue-derived material at the target site may be controlled by altering the release properties of the hydrogel. In some embodiments, the release properties may be engineered by controlling one or more of the expression of extracellular matrix or protein motifs, the presence or absence of fusion proteins, the net charge of the peptides, the presence or absence of cationic particles or peptides, the presence or absence of anionic particles or peptides, buffers, salts, peptide concentration, peptide purity, and the presence or absence of peptide counterions. The properties may be engineered to deploy cells at the target site. The properties may be engineered to deploy cell products or byproducts at the target site, for example, delivery of exosomes, growth factors, genetic material, RNA, siRNA, shRNA, miRNA, etc.

The self-assembled hydrogel may be designed to have cell protective properties. In particular, the self-assembled hydrogel may be designed to be protective against foreign microorganisms, e.g., pathogenic microorganisms. The self-assembled hydrogel may be designed to be protective against immune attack from environmental immune cells, for example, by providing a physical barrier or biochemical modulation. The antimicrobial and/or protective properties of the hydrogel may not substantially affect the viability, growth, or function of the engrafted cells.

The protective properties of the hydrogel may be engineered by altering the net charge of the peptides. In some embodiments, the net charge may be altered by controlling one or more of the presence or absence of cationic particles or peptides, the presence or absence of anionic particles or peptides, buffers, salts, peptide concentration, peptide purity, and the presence or absence of peptide counterions.

The suspension may be designed to have a substantially physiological pH level. The suspension may have a pH level of between about 4.0 and 9.0. In some embodiments, the suspension may have a pH level of between about 7.0 and 8.0. The suspension may have a pH level of between about 7.3 and 7.5. The substantially physiological pH may allow administration of the suspension at the time of preparation. In some embodiments, the suspension may be prepared at a point of care. The methods may comprise suspending the cells in the peptide solution, optionally, agitating the suspension to provide a substantially homogeneous distribution of the cells, and administering the suspension at a point of care. The administration may be topical or parenteral, as described herein.

Biofabrication of Biological Material Grafts with Peptide Hydrogels

Methods of preparing biological material grafts in vitro for administration in vivo are disclosed herein. The methods may include self-assembly of a liquid suspension comprising cells into a peptide scaffold matrix in vitro. The self-assembled higher order structure may be administered to a target site of the subject.

Methods of preparing biological material grafts in vivo are disclosed herein. The methods may comprise administering a liquid suspension comprising the biological material for self-assembly into a higher order structure at a target site.

The methods may include biofabrication of the biological material graft at a point of care, as described in more detail below.

The hydrogels disclosed herein have gelation kinetics which are fast enough to ensure biological material becomes substantially homogeneously incorporated within the matrix. In particular, the gelation kinetics are sufficiently fast to afford an even distribution of encapsulated cells to allow reproducible control over cell density within the matrix. Additionally, the hydrogels disclosed herein have a construct that can be introduced in vivo and remain localized at the point of administration, for example, even without a spatial cavity. The localization of the hydrogel upon administration can limit or inhibit leakage of the cell construct to neighboring tissue.

The methods may comprise suspending the biological material in the preparation, optionally, agitating the suspension to provide a substantially homogeneous or non-homogeneous distribution of the biological material, and administering the suspension at a point of care. In some embodiments, the suspension may be agitated to provide a substantially homogeneous distribution of the biological material. In other embodiments, the suspension may be prepared or agitated to provide a non-homogeneous suspension, for example, comprising clusters or spheroids of the biological material.

Prior to engrafting, the cells may be cultured in vitro. Cell culture protocols generally vary by cell type. The conditions of the cell culture protocol may be selected based on the cell type. In exemplary embodiments, the cells may be autologous, allogeneic cells, or xenogeneic cells. The cultured cells may be suspended in water and/or media. In some embodiments, the methods disclosed herein may comprise collecting or harvesting cells from an organism. For instance, the methods disclosed herein may comprise collecting or harvesting cells from the subject. The methods disclosed herein may comprise collecting or harvesting a tissue graft from an organism, e.g., the subject.

The suspension may include a peptide configured to self-assemble in response to an external stimulus. The suspension and/or peptide may be engineered to express a desired property. For example, the suspension and/or peptide may be designed to express shear-thinning and/or antimicrobial properties.

In some embodiments, a buffer solution may be added to the suspension or a portion of the suspension to induce hydrogelation prior to or concurrently with administration. The hydrogel may form a homogenous or substantially homogenous cell matrix. The cell matrix may be administered to a target site as a solid or gel, optionally with shear-thinning properties as previously described.

The cells may be cultured in the cell graft in vitro for a predetermined period of time prior to administering the cell suspension to the subject. The period of time may range from several minutes, to several hours, to several days. The culture period may be selected based on cell type and target application. In other embodiments, the cells may be administered immediately after suspending or engrafting. The cells may be cultured in situ in the implanted cell graft.

The suspension and/or peptide may be engineered to express a desired property. In certain embodiments, the suspension and/or peptide may be engineered to protect cells from hostile environments. In particular, the suspension and/or peptide may be engineered to protect the cells from environments with a high microbial burden, hostile immune cells, or environmental proteins. The suspension and/or peptide may be engineered to increase cell viability. The suspension and/or peptide may be engineered to control differentiation, control cell fate in situ, control cell fate in vivo, control cell fate ex vivo, or control cell fate in vitro. The suspension and/or peptide may be engineered to increase cell attachment to the matrix or increase cell attachment and/or migration in the environment. The suspension and/or peptide may be engineered to decrease apoptosis, for example, by providing cell attachment and/or biological modulation.

The suspension and/or peptide may be engineered to achieve the results described above by altering the expression of protein motifs or the net charge of the peptides. The hydrogel properties may be engineered by controlling one or more of the expression of extracellular matrix or protein motifs, the presence or absence of fusion proteins, the net charge of the peptides, the presence or absence of cationic particles or peptides, the presence or absence of anionic particles or peptides, buffers, salts, peptide concentration, peptide purity, the presence or absence of peptide counterions, the presence or absence of specialized proteins, and the presence or absence of specialized small or large molecules.

Mixing devices for biofabrication of cell grafts at a point of care are disclosed herein. The devices may include a first chamber for a cell preparation. The cell preparation may comprise cells suspended in water, media, or buffer. The devices may include a second chamber for a peptide preparation, and optionally a third chamber for a buffer, as previously described.

### EXAMPLES

The function and advantages of these and other embodiments can be better understood from the following examples. These examples are intended to be illustrative in nature and are not considered to be limiting the scope of the invention.

### Example 1: Broad-Spectrum Antimicrobial Effect of the Peptide Preparations

It has been shown that cation-rich peptides exhibit antimicrobial properties. Additionally, hydrogels rich in arginine have shown a greater antimicrobial effect toward drug-resistant bacteria (S. aureus) than those rich in lysine. Testing both arginine-rich and control lysine-rich hydrogels with methicillin-susceptible and methicillin-resistant S. aureus (MSSA and MRSA), it was found that both gels cleared MSSA at all doses tested by 99.9% (FIG. 4).

As shown in FIG. 4, in comparison to tissue culture polystyrene (TCPS) (.) and lysine control gels (o), arginine-rich gels (Q) were able to inhibit bacterial proliferation, even when challenged with increasing numbers of CFUs of MRSA (Graph A of FIG. 4) and MSSA (Graph B of FIG. 4). Both lysine-rich and arginine-rich hydrogels effectively eliminated more than 99.9% of bacteria at low doses of MRSA (<10⁴ colony forming units CFU/dm²). However, only arginine-rich hydrogels were able to effectively eliminate high densities of MRSA (>10⁸ CFU/dm²). The data show improved results with arginine-rich peptides. Additionally, the results suggest that the breadth and specificity of the hydrogel can be engineered by altering the sequence of individual peptides.

The ratio of lysine and arginine was varied, as shown by the exemplary peptide sequences in FIG. 5. The antimicrobial activity of the exemplary hydrogels (PEPXR) was evaluated against both gram-positive and gram-negative bacteria, including E. coli (Graph B of FIG. 5), S. aureus (Graph C of FIG. 5) and P. aeruginosa (Graph D of FIG. 5).

The antibacterial assays showed that the peptide hydrogels are effective at reducing the viability of these bacterial strains to less than 0.01%. In particular, at least 4 arginine residues among a background of lysine residues demonstrated potent activity. Thus, the peptide hydrogels described herein may generally comprise a high content of positively charged arginine (R) amino acids. For instance, exemplary peptide PEP6R having a sequence comprising VKVRVRVRVPPTRVRVRVKV showed potent antimicrobial activity.

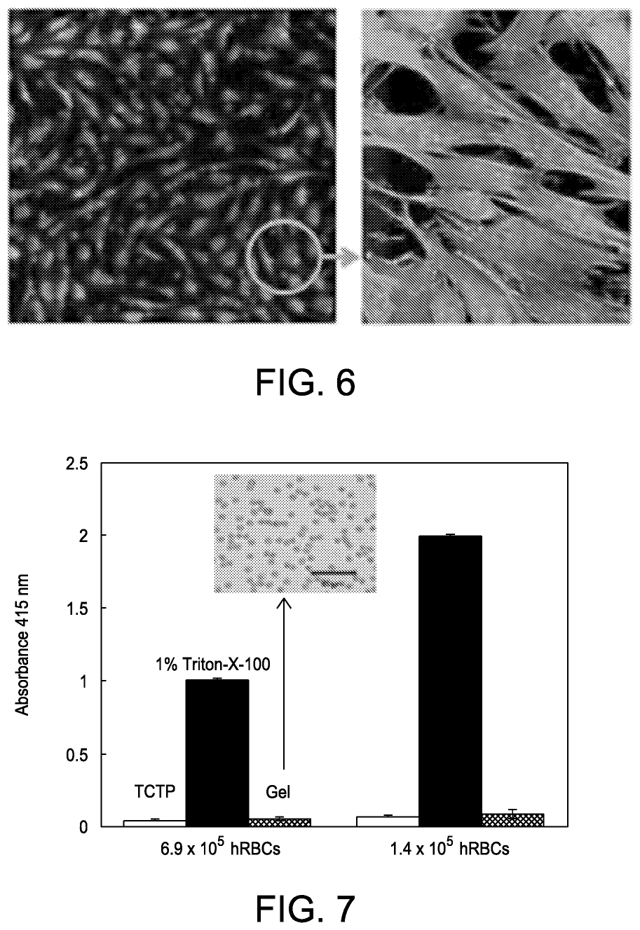

While not wishing to be bound by theory, it is believed the repeat display of positive charges on the hydrogel surface can directly lyse negatively charged bacterial membranes, thereby killing bacteria without needing to add extraneous antibacterial agents. Although high arginine content increases the hydrogel's bacterial membrane lysing potential, it is beneficial to balance the lysine with arginine amino acids to preserve host cells. It has been demonstrated that modifying the ratio of arginine to lysine can tune the hydrogel's biocompatibility while preserving bactericidal effects. For example, seeding with both E. coli and mammalian cells simultaneously onto the hydrogel showed that bacteria were killed while mammalian cells remained >90% viable after 24 hours and capable of proliferation. FIG. 6 is a magnified image of live/dead cells after an assay of co-cultured MRSA and C3H10t1/2 mesenchymal stem cells on the peptide hydrogel. As shown in FIG. 6, MRSA was killed while mammalian cells remained healthy.

Furthermore, the charge density of amino acids and net charge of the tested peptide sequences was shown to be compatible with erythrocytes and macrophages (FIGS. 7-8). Briefly, FIG. 7 shows hemolytic activity of a TCTP control surface, Triton-X control, and a 2 wt % antimicrobial hydrogel after 5 hours of incubation. FIG. 8 shows a laser confocal scanning microscopy (LSCM) image of a live/dead cellular viability assay of 20,000 J774 macrophages cultured on the 2 wt % peptide gel surface after 48 hours.

The peptide hydrogel is believed to directly lyse bacterial membranes indiscriminately. Therefore, the peptide hydrogels are suitable for eliminating and/or inhibiting a wide range of bacterial microorganisms (for example, gram-positive and gram-negative). The mechanism additionally makes it more difficult for the treated microorganisms to gain antibiotic resistance. The peptide hydrogels have demonstrated effective treatment of both gram-positive and gram-negative bacteria, including multi-drug resistant bacteria, such as P. aeruginosa, with gels at 1.5 wt % peptide concentration.

Additionally, the lysine-rich peptide hydrogels have demonstrated biocompatibility in vivo through subcutaneous implantation in mice. Histology was analyzed after 7 days at the hydrogel implantation site. No cellular debris or dead cells were observed. Cytokine array analysis of the hydrogel and surrounding tissue revealed little increase over naïve mice tissues, suggesting the hydrogel has insignificant acute inflammation effects. Arginine-rich peptide hydrogels are expected to show similar biocompatibility in vivo.

### Example 2: Proposed Tests to Show the Prevention of Surgical Site Skin Infections by Intra-Tissue Administration of the Antimicrobial Surgical Wound Matrix

It is proposed that the results from in vitro lab studies can be robustly extended to prevention of surgical site infections. The methodology proposed includes demonstrating the manufacture of the hydrogel, evaluation of efficacy, and verification of biocompatibility. The tests will focus on generating robust feasibility data using a combination of a large animal model (histopathology of dermal wound) and a small animal model (microbiology of abdominal wound infections).

A tissue cytokine array of lysine-rich hydrogel and fibrotic tissue on days 3, 7, and 30 compared with tissue samples from naïve mice showed no significant increase in inflammatory effects.

In a rheological study, gelation was initiated by combining the peptide preparation with a buffer. The gel was allowed to form for 1 hr. Subsequently, 1000% strain was applied for 30 seconds, showing shear-thinning. The gel was recovered over the next hour.

### Research Design: Therapeutic Safety

In vivo safety of the peptide preparations will be examined during wound healing. The objective of this specific aim is to demonstrate that hydrogel administration into incisional dermal wounds in a swine model will not impair tissue healing measured at day 14 by performing histopathological analysis of the healing wound tissue. Safety performance by qualitative histopathology (fibrotic tissue thickness and giant body response around gel tissue interface) will be compared to PBS controls and comparator product at day 14.

All hydrogel formulations and fill finish will be made at 4% (wt/v) under aseptic conditions and diluted to test concentrations of 0.75%, 1.5%, and 2.5% wt/v by adding sterile water. Manufacturing specifications of peptides have been defined and will be confirmed to peptide purity of >95%, endotoxin <1 EU/mg. Peptide hydrogels used in this study will be arginine-rich peptides. Materials will be made ready to use for in vitro and in vivo studies. Aseptic formulation and fill finish (0.2 μm filtering) of the pre-clinical product will be performed.

40-50 kg female domestic pigs will be used as the in vivo wound model due to their morphological similarities to human skin and because wound healing models have been well characterized. A time point of 14 days is selected to determine gel safety performance during incisional wound healing (transition from granulation to remodeling phase) and by-products of gel degradation during wound healing.

One pig with twenty-five 3 cm full thickness incisional wounds (including skin, fascia, and muscle) will be created in a dorsal paraspinal orientation. One ml of test articles will be administered into incisional wounds. The wound will be closed with a 7.0 suture, each loop 0.6 cm apart, and covered with a non-adherent secondary dressing (3M-Tegaderm). Five wounds/group will be implanted with 1 ml of the treatment of either PBS or peptide preparation (PEP6R at 1.5% w/v; PEP6R at 0.5% w/v; PEP6R at 2.5% w/v, and comparator product (Gentamicin Collagen).

Photographs of the wounds will be documented at days 1, 3, 7, and 14 for visual evaluation of pus, redness, swelling, exudate, crusting, inflammation, and scab tissue. At day 14, animals will be euthanized, and tissues and gels will be excised. The samples will be fixed in 10% neutrally buffered formalin and analyzed using standard histology. Tissue samples will be compared to control for tissue morphology and necrosis. Gel samples will be analyzed for any fibrotic tissue and cell infiltration. Fibrotic tissue thickness will be quantified as one indication of inflammation. Cell necrosis will be quantified as one indication of toxicity. Wound healing will be scored by a board-certified wound healing pathologist for bridging, scarring, re-epithelialization, granulation, edema, erythema, gel integrity, and macrophage/giant body cell response (cell/cm²). Left over tissue biopsy samples will be stored for analysis of inflammatory markers.

It is expected the study will determine the material is safe during incisional wound healing. The swine study design allows for multiple comparisons of treatment interactions in a single system, reducing possible variability when assessing differences between treatments.

Additional studies will be performed to on additional time points, infection related characterization of wound healing, wound integrity quantified by tensile testing, wound hemostat capabilities, and local and distant toxicity studies.

The study will provide histopathology and visual scoring reports. The hydrogel is expected to be considered safe and provide no local inflammation, fibrosis, or skin sensitivity observed in the animals. It is expected that the product hydrogel will be determined to be safe and biocompatible through the implantation period.

Histopathology results of peptide preparation will be compared to low (0.75% w/v) and high concentrations (2.5% w/v) of peptide, comparator product, and PBS controls. Less than 10% necrotic cells are expected to be observed within the hydrogels at day 14. Degradation products at week 2 are not expected to have induced large macrophage and giant body cell response (quantified by cell/cm²). The safety of the validation study is expected to be demonstrated through histopathology by showing that the hydrogel does not elicit significant acute or chronic inflammation or toxicity in vivo.

### Research Design: Therapeutic Efficacy

Therapeutic in vivo efficacy of infection prevention will be studied. The study will demonstrate the therapeutic in vivo efficacy of the hydrogel to effectively clear bacteria from infected abdominal incisional wounds with less than 10³ CFU/g wound as quantified by bacterial titers at days 1 until 5.

To demonstrate infection elimination efficacy, abdominal incisional wounds infected with a MRSA inoculation of 10⁸ CFU/ml will be tested for bacterial presence at day 1 and 5 after treatment. The study will demonstrate that the preparations can effectively clear bacteria in infected wounds with less than 10³ CFU/g wound as quantified by bacterial titers and comparing average log 10 CFU/g wound of the treated groups.

A rat abdominal wound model for infections will be used for the study. Treatment groups for the study are: (i) Inoculate (I)+PBS control (efficacy control), (ii) No Inoculate (NI)+PBS control (safety control) (iii) I+peptide preparation treated (efficacy test), (iv) I+comparator product (Gentamicin Collagen) (control for comparator performance). Outcomes will be measured at time points of 1 and 5 days post-injury.

5 animals per group will be tested. Each animal will be tested with one 3.0 cm full-thickness abdominal wall incision (exposing the viscera) in the ventral midline (i.e., through the linea alba), beginning just below the xiphoid process and extending inferiorly. The incision will be closed following the steps: (i) fascia with continuous 5-0 Vicryl (Ethicon Inc, Somerville, NJ) suture, (ii) a single dose of MRSA (ATCC #33591, 108 CFU/ml at a defined volume (i.e., 0.01 mL) will be applied to the wound allowed to sit undisturbed for 15 minutes before treatment (post infection), (iii) 0.1 ml of treatment will be applied, and (iv) dermal layer will be closed with continuous sutures (each incision will contain 20 suture loops with 1 mm distance apart). No dressing will be applied, and no antibiotics will be used unless as treatment group.

Pre and postoperative observations will be documented qualitatively for hydration, crusting, exudate, and manageability. At days 1 and 5, animals from each group will be humanely euthanized. The anterior abdominal wall will be excised with all layers from the costal margins superiorly, out to both extreme flanks laterally, and down to the hindquarters inferiorly (as shown in FIG. 9). This specimen contained the linea alba, which bisected the specimen in the vertical direction (FIG. 9). A 30 mm by 30 mm rectangle of abdominal wall was excised from the central portion of this specimen (FIG. 9), and then cut into two strips, each 30 mm by 15 mm (FIG. 9). One strip will be used for microbiological analysis and the other for histopathological analysis.

For microbiological analysis, the tissue strips will be homogenized and analyzed for number of viable bacteria. Data analysis will consist of numbers of CFU/g tissue for each group and comparing average log 10 CFU/wound of the treated groups to the infection control group. Furthermore, to test full elimination of the bacteria, samples will undergo serial dilutions plated on nutrient rich agar. Resulting colonies will be quantified. For histopathological analysis, the wound tissue strips will be fixed in 10% neutral buffered formalin and processed for standard qualitative histopathological analysis using hematoxylin-eosin and tissue gram staining for localization of bacteria in the wound tissue and around gel, fascia integrity, dermal integrity (bridging, re-epithelialization, granulation, edema, erythema, biocompatibility and giant body cell response).

A study will be performed on quantitative microbiology to determine wound bioburden and qualitative histology. The study will show effective clearing of a MRSA from wounds to less than 10³ CFU/g wound as quantified by bacterial titers comparing average log 10 CFU/g of wound tissue by the peptide experimental group at days 1 and 5. Left over tissue biopsy samples will be stored for analysis of inflammatory markers.

Fifteen rats have been allocated for model development. Preliminary results have shown that the peptide solution clears by at least 99.9% at high dosage MRSA colony (>108 CFU/dm2) within 24 hours in vitro. Therefore, it is anticipated that the peptide preparations will eliminate MRSA from wounds by at least 10³ CFU/g wound by 24 hours and maintain the effect until 5 days.

It is possible that the endotoxins from bacterial lysate will cause inflammation. Such effects will be compared to PBS control groups, and comparator, for a clear interpretation on the side effects of bacterial elimination using qualitative digital photographs and histopathology (giant cells/cm2 tissue). Data will also be gathered on wound tissue integrity and healing capacity, which will further provide validation of the peptide preparations.

MRSA will be used as an example of a clinically relevant MDRO. However, studies on other MDRO's are expected to produce similar results. Further in vitro studies will screen for a large set of clinically relevant gram positive and gram negative bacteria to demonstrate hydrogel antimicrobial activity.

Using a stringent therapeutic infected wound model and high inoculations of 10⁸ CFU/ml, it is expected that the peptide preparations will effectively eliminate pathogens by 4-fold when administered as a hydrogel.

An alternate strategy to reduce the risk of infection is to combine administration of the peptide hydrogel with systemic antibiotics in the animal models and reduce the bacterial load. MRSA at 10⁶ CFU/ml will be added around the edges of a wound after administration of hydrogel to determine efficacy. The results will demonstrate that the peptide hydrogel can prevent the growth of bacteria in surgical wounds.

### Example 3: Antimicrobial Properties of MSC Engrafted Peptide Hydrogels

The study will demonstrate that MSCs encapsulated in antimicrobial peptide matrices will enhance tissue regeneration of full-thickness wounds in db+/db+diabetic mice. The db/db mouse model of diabetes (db+/db+; BKS.Cg-Dock7m+/+Leprdb/J) is a commonly used model of diabetic wound healing that exhibits vulnerability to infections, altered host response, and impaired healing. Tissue morphology between the different treatment groups at day 14 (partial wound closure) and day 28 (complete wound closure) for clean diabetic wounds and at day 28 for pathogen-contaminated wounds, which are expected to exhibit delayed tissue regeneration will be examined. The study will demonstrate that the peptide matrices delivering MSCs lead to accelerated rate of wound closure and improved quality of regenerated tissue as compared to controls.

The study will demonstrate accelerated tissue regeneration in clean diabetic wounds. In this study, it will be demonstrated that the hydrogel matrices can improve healing of non-infected wounds in db+/db+ mice. A total of 30 female 10-week old db+/db+ mice will each receive two full thickness skin wounds, following which they will be randomized into the following treatment groups: 1) PBS (control), 2) 0.5×10⁶ MSCs only (control), 3) collagen scaffold+0.5×10⁶ MSCs (comparator product control), 4) self-assembling peptide hydrogel, and 5) self-assembling peptide hydrogel+0.5×10⁶ MSCs.

Animal surgeries will be performed according to previously established protocols. Briefly, mice will be placed under anesthesia and two 5-mm full thickness excisional wounds will be created on the dorsum of each mouse, on either side of the midline. Donut-shaped silicone splints will be placed around the wounds and affixed using liquid adhesive (Krazy® Glue, Elmer's Products) and interrupted sutures. 100 μL of treatment will be applied to the wounds before covering with Tegaderm™ (3M), a non-adherent wound dressing. For the MSC only control, MSCs suspended in 100 μL of PBS will be injected intradermally at 4 sites in the periphery of the wound. For the comparator product control, MSCs will be pre-seeded onto collagen scaffolds (Integra® wound matrix or other similar porous collagen scaffold) for 30 min at 37° C. prior to placement of the scaffold in the wound bed. Wounds will be photographed at days 0, 3, 7, 14, 21, and 28 in order to measure the wound surface area and quantify the percent wound closure over time by image analysis.

When the wound dressings are removed, wounds will be scored (wound scoring, Draize scoring for skin irritation) and qualitatively assessed for hydration, crusting, exudate, and manageability. Mice will be euthanized at days 14 and 28, and the wounds and surrounding tissue will be excised using 10 mm biopsy punches. 6 wounds per condition will be processed for H&E staining, and histological sections will be evaluated for re-epithelialization, granulation tissue formation, edema, erythema, fibrotic tissue, and giant body cell response (quantified by cell/cm²). The study will establish accelerated rate of wound closure and improved quality of regenerated tissue (increased re-epithelialization and granulation tissue formation) in treated groups compared to controls.

Preliminary in vitro results have shown that the peptide hydrogels are cytocompatible, suggesting that the gels can both support the survival and function of exogenously delivered MSCs, as well as allow invasion and proliferation of endogenous cells for tissue regeneration. It is anticipated that the peptide hydrogels delivering MSCs will show improved healing of diabetic wounds compared to controls, as measured by the rate of wound healing (by image analysis) and histopathology assessments by a board certified pathologist.

The peptide hydrogels can mediate cell attachment (FIG. 6) to support the viability and function of encapsulated MSCs, as well as serve as a scaffolding matrix for infiltration of endogenous cells during the wound healing process. FIG. 6 includes images demonstrating selective toxicity against pathogens. In particular, FIG. 6 shows live cells (green) and dead cells (red) on an assay of co-cultured MRSA and C3H10t1/2 mesenchymal stem cells on the peptide hydrogels. As shown in the right image of FIG. 6, MRSA is killed while mammalian cells remain healthy. Higher magnification shows C3H10t1/2 cells are able to adhere and spread on the hydrogel.

Bioactive peptide hydrogel effects on enhanced cell attachment capacity and wound healing will be examined. In addition, bioactive peptide matrices have the potential to synergize with MSCs through their biological activity to accelerate wound healing.

The peptide hydrogels may accelerate tissue regeneration in pathogen contaminated diabetic wounds. The inherent antimicrobial properties of the peptide hydrogels will be determined to protect therapeutic MSCs encapsulated within the matrix following delivery into infected diabetic wounds, enabling improved wound healing. Wounds will be created in 20 db+/db+ mice as described above. After wound creation and splint application, 105 CFU of S. aureus (ATCC 25923) in 10 μl of PBS will be placed on the wound bed and allowed to sit undisturbed for 15 minutes. Following inoculation, 100 μl of treatment will be applied before covering with Tegaderm wound dressing.

Treatment groups will consist of: 1) PBS (control), 2) 0.5×10⁶ MSCs only (control), 3) collagen scaffold+0.5×10⁶ MSCs (comparator product control), 4) self-assembling peptide hydrogels, and 5) self-assembling peptide hydrogels+0.5×10⁶ MSCs. The rate of wound closure will be monitored at days 3, 7, 14, 21, and 28 by digital photographs, and wounds will be scored following identical methods as described above. Following euthanasia at day 28, wounds will be excised using 10 mm biopsy punches. 8 wounds per treatment group will be processed for H&E staining and subjected to histopathology assessments. The study will establish accelerated rate of wound closure and improved quality of regenerated tissue (increased re-epithelialization and granulation tissue formation) in the groups treated with the peptide hydrogels compared to controls.

Preliminary in vitro results have shown that the peptide hydrogels exhibit potent antimicrobial effects, while simultaneously remaining cytocompatible with mammalian cells. Therefore, it is anticipated that the peptide hydrogels delivering MSCs will improve the healing of infected wounds in diabetic mice as compared to control treatments, as measured by the rate of wound healing (by image analysis) and histopathology assessments by a board certified pathologist. The parallel treatment groups examined as described above will serve as uninfected wound healing controls to compare against the infected wounds studied here.

Bioburden in wounds may be measured using tissue biopsies (vs. swabs) between days 1-7. Healing phenotype may also be assessed. Wound bioburden in the treatment groups will be compared at days 3 and 7 by taking wound biopsies and quantifying the bacterial burden (CFUs/g of tissue). Complementary and ongoing studies will test a larger set of clinically relevant pathogens.

Feasibility of peptide hydrogels will be demonstrated by confirming in vivo viability of transplanted MSCs encapsulated in peptide hydrogels, and improved tissue regeneration in vivo in clean and contaminated diabetic wounds following treatment with MSCs encapsulated in the peptide hydrogels. Following, the efficacy of the peptide hydrogels for promoting tissue regeneration in a diabetic swine model will be determined. The study will consist of topically applied therapeutic cells encapsulated in synthetic matrices.

### Example 4: Microbial Pathogen Elimination by Administration of Peptide Preparations

A study was performed to determine the therapeutic in vivo efficacy of the peptide preparation to eliminate infection from a wound in a mouse model infected with methicillin-resistant S. aureus (MRSA). The study demonstrated superior clearing of MRSA from wounds by days 1 and 3. Administration of the peptide preparation led to significant bioburden reduction in vivo compared to untreated controls and comparator product controls at various inoculation concentrations. First, in vitro bacterial clearance was validated. Then, safety and biocompatibility of the hydrogel were confirmed, and antimicrobial efficacy was demonstrated in vivo.

The peptide hydrogels were successfully manufactured to specifications, with endotoxin levels <0.05 EU/ml. Further work will be performed to demonstrate GMP grade synthesis and product shelf stability.

The peptide hydrogel's potent antimicrobial activity against model organisms was first confirmed in vitro. An in vitro antimicrobial activity assay was performed by seeding test organisms directly on an agar plate, then treating the inoculated spots with antimicrobial gels. The hydrogel demonstrated 6 log CFU reduction of MRSA, exceeding the FDA criteria of ≥4 log reduction for antimicrobial effectiveness. Comparator controls did not effectively control MRSA in the assay. Comparator controls Medihoney® Gel (McKesson Brand, Irving, TX), Anasept® Gel (Anacapa Technologies, Inc., San Dimas, CA), and Silver-Sept® Gel (Anacapa Technologies, Inc., San Dimas, CA) showed <2 log reduction. Consistent with these findings, the peptide hydrogel was also observed to exhibit bactericidal activity.

To evaluate biocompatibility in vivo, mice were injected subcutaneously with the peptide preparation, PBS (procedure control), and product comparators (implant controls) including Stimulen™ collagen gel (Southwest Technologies, Kansas City, MO), Medihoney®, and Silver-Sept®. The administered materials were well tolerated except Medihoney® (2 of 5 mice injected with Medihoney® died after administration).

To characterize different phases of the inflammatory response, day 3 animals were assessed for acute reactions and day 14 animals were evaluated for chronic inflammation. As expected, all materials were associated with similar early inflammatory responses. On day 3, the inflammatory cells around the implants were primarily neutrophils, with some lymphocytes and macrophages. On day 14, the peri-implant inflammation was chronic (with primarily macrophages and lymphocytes). On day 14, neutrophils and fibrosis were observed in some mice. PBS controls confirmed the implantation procedure causes minimal reaction.

The results were within the expected range for a degradable biomaterial. It should be noted that the preparation hydrogels used in the study were not fabricated using GMP grade materials. However, the product comparators all met GMP standards since they were over-the-counter commercial products. It is expected that GMP grade preparation hydrogels will confirm similar or superior safety and biocompatibility than the comparator products tested.

Additionally, the experimental preparation was the only treatment that resulted in a statistically significant reduction in wound bioburden relative to the untreated controls. Preliminary data suggested that the hydrogel could reduce bioburden in vivo in wounds inoculated with MRSA or P. aeruginosa. A study was conducted to confirm the preliminary findings.

Briefly, full-thickness dermal wounds (5 mm diameter) were created bilaterally in CD-1 mice. The wounds were inoculated with MRSA (ATCC #33591) at indicated doses for 15 min. At various timepoints, a minimum of 8 wounds and surrounding tissue were excised, homogenized, and analyzed to quantify bioburden as log 10 CFU/g of tissue (* p<0.05, ** p<0.01, *** p<0.001, **** p<0.0001, ANOVA with corrections for multiple comparisons).

Wound bioburden was examined at 6, 24, and 72 hours following inoculation with MRSA at 10⁴, 10⁵, or 10⁶ CFU/wound and treatment. Wounds were treated with PBS control (untreated), 100 μL of peptide preparation at 0.75% w/v, collagen (Stimulen™), Medihoney®, and Germ Shield™ silver gel (Curad, Northfield, IL).

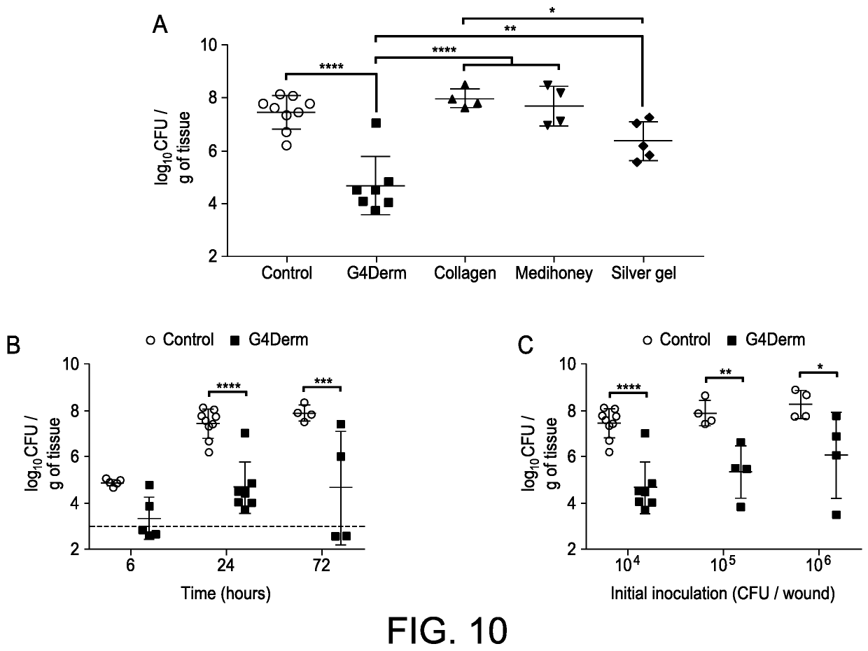

The data is presented in the graphs of FIG. 10. Wound bioburden in the hydrogel (labeled G4Derm) treated condition was significantly lower than in every other condition tested, which included an untreated control and 3 product comparators collagen (Stimulen™), Medihoney®, and Germ Shield™ silver gel. The hydrogel was the only treatment that resulted in a statistically significant reduction in wound bioburden relative to the untreated controls (2.8 log reduction; as shown in the graph (A) of FIG. 10). As expected, regenerative matrix collagen scaffold appeared to increase pathogenic colonization. The bioburden reduction in wounds treated with the preparation hydrogel was observed at all timepoints examined (6, 24, and 72 hours). At 6 and 72 hours, several of the peptide hydrogel treated samples were below the detection limit for this assay (as shown in graph (B) of FIG. 10). Furthermore, the peptide hydrogel was significantly effective at reducing wound bioburden in all the conditions tested, ranging from 10⁴ to 10⁶ CFU/wound (graph (C) of FIG. 10). The outliers are also presented. As expected during monitoring, when the peptide dressing was removed by animal aggression, the bioburden was no longer controlled resulting in outliers.

### Example 5: Peptide Preparations Administered for Promoting Wound Regeneration

A study was performed to determine the efficacy of the peptide hydrogels at promoting wound regeneration. The study demonstrated superior in vivo wound healing by 14 days. In particular, the hydrogel provided superior wound healing relative to controls. Two of the wounds treated with the peptide hydrogel showed complete wound closure. No other treatments exhibited full re-epithelialization.

Briefly, 2 cm full thickness dermal wounds were created in pig animal models. The wound beds were treated with PBS, preparation hydrogel (at 0.75% w/v and 1.5% w/v), and product comparators including collagen (Stimulen™) and Germ Shield™ silver gel. 4-5 wounds were treated with each treatment model. At day 0, 500 μL of treatment was applied to each wound and covered with Tegaderm™ transparent wound dressing (3M, Saint Paul, MN). At day 7, the wounds were treated with an additional 200 μL of treatment and re-covered with Tegaderm™.

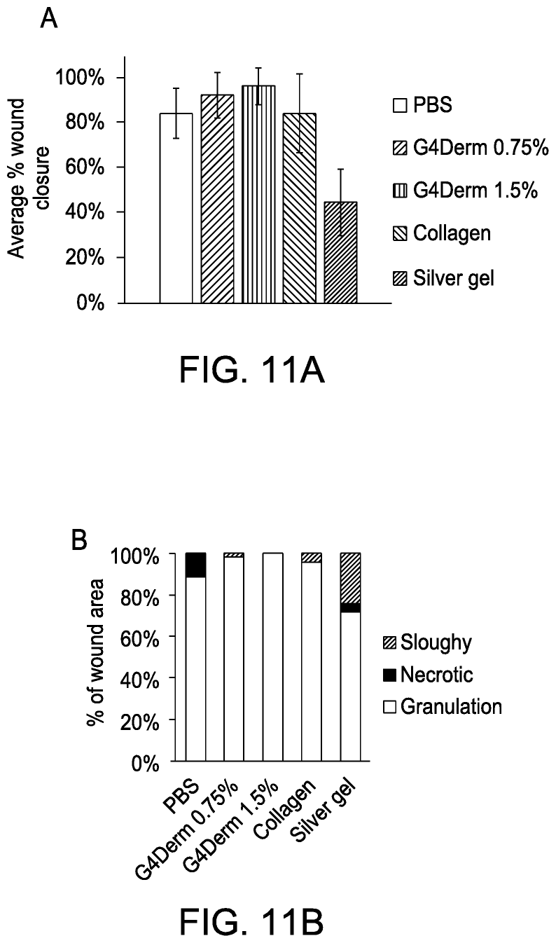

The graph of FIG. 11A shows wound closure at day 14 expressed at percent wound closure (determined by measuring and calculating re-epithelialized area divided by initial wound area). The graph of FIG. 11B shows wound analysis by distinguishing granulation tissue from necrotic and sloughy tissue at day 14. Different tissue types were quantified by image analysis and represented as a percent of the total wound area. The hydrogel treated wounds demonstrated the least necrotic and slough tissue.

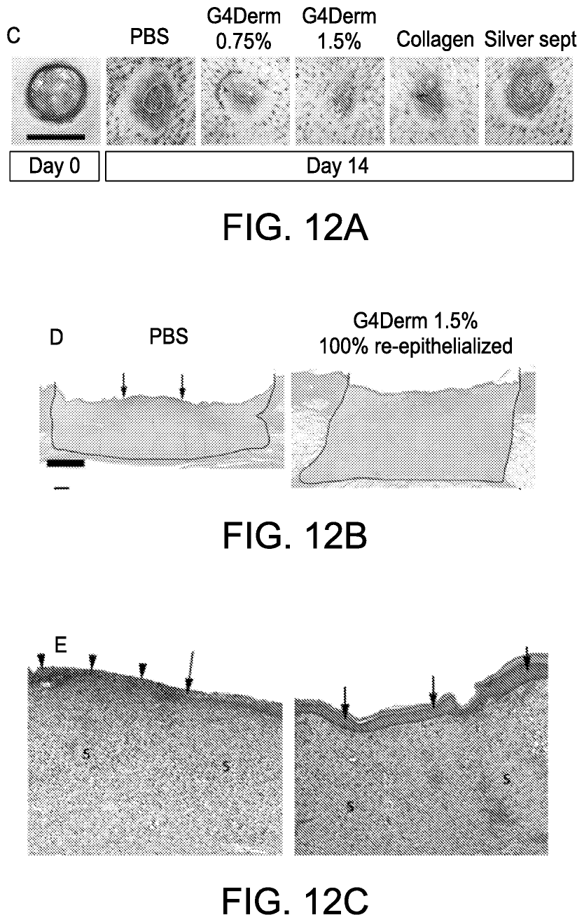

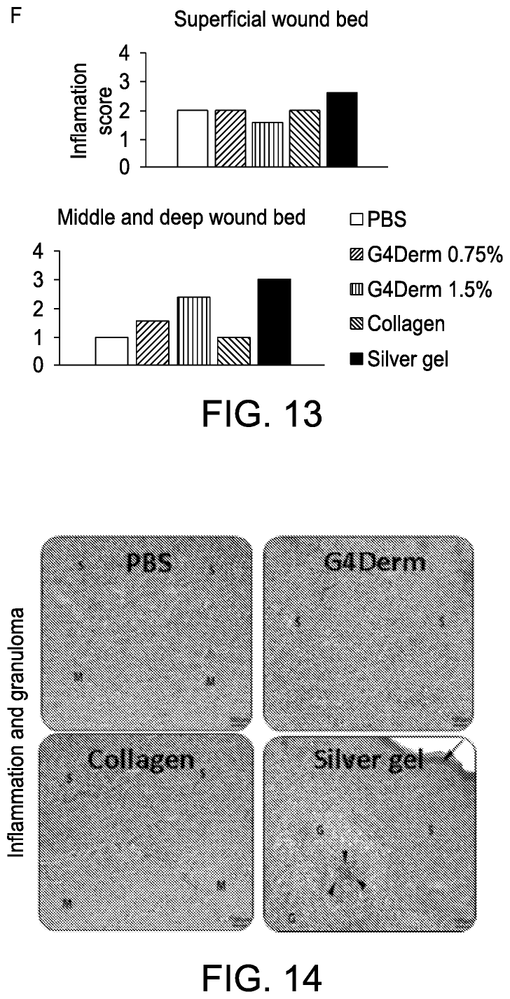

The images of FIG. 12A show wounds at day 0, after creation of an injury, and wounds at day 14 for each treatment method. Blue lines outline the periphery of the ulcerated, non-epithelialized wound area. FIGS. 12B, 12C, and 14 show images of H&E stained tissue sections. FIG. 12B shows a PBS treated wound (left; arrows=edges of epithelial ingrowth) and a hydrogel (1.5%) treated wound (right; fully epithelialized). The solid line marks wound borders. FIG. 12C shows higher magnification views of images in FIG. 12B, showing regions in the center of the wound. The PBS treated wound (left) had partial epithelialization (arrow=edge of epithelial ingrowth) and ulceration in center of the wound (arrowheads). The preparation hydrogel treated wound (right) had epithelium completely covering the surface of the wound bed (scale=200 μm). Tissue sections were scored for inflammation in different regions of the wound bed (0=not present, 1=minimal, 2=mild, 3=moderate, 4=marked, 5=severe). The data is presented in the graph of FIG. 13. Overall, there were no adverse responses due to implantation (under score 3). The images of FIG. 14 show granuloma formation. Granuloma formation was similar (minimal) in all test groups except Germ Shield™ silver gel, which showed an elevated inflammatory response around residual test material (indicated by arrows).

Wound image analysis was performed using a clinical platform by Tissue Analytics, Inc. (Baltimore, MD) to quantify wound closure at days 7 and 14. No difference was seen between collagen matrix and PBS groups, each of which achieved 84% closure at day 14. Surprisingly, antimicrobial comparator Germ Shield™ silver gel delayed wound closure, resulting in 44% wound closure by day 14. The peptide hydrogel treated samples showed the greatest extent of wound closure, with averages of 92% and 96% wound closure in the 0.75% w/v and 1.5% w/v treated samples, respectively (as shown in the data presented in FIGS. 11A-11B and the images of FIG. 12A).

Additionally, different types of wound tissue were characterized by image analysis. As shown in the data presented in FIG. 11B, at day 14 the majority of wound area treated with the hydrogel preparation consisted of healthy granulation tissue, with no signs of necrotic or sloughy tissue present. The wounds treated with Germ Shield™ silver gel had less than 50% granulation tissue, with a large proportion of necrotic tissue.

Histology analysis was performed on wound tissues after 14 days. The tissue sections were evaluated for granulation tissue, re-epithelization, and inflammatory response to implants. The experimental group was the only treatment group in which complete wound closure was observed, with 2 of 5 wounds treated with the peptide hydrogel achieving complete re-epithelialization in 14 days (shown in images of FIGS. 12A and 12C).

Inflammation in different regions of the wound bed was assessed by histology. The data is presented in the graph of FIG. 13. Inflammation was similar in all test groups (scored under 3 for minimal) except Germ Shield™ silver gel treated groups. The Germ Shield™ silver gel treated groups showed the highest level of inflammation with granulomas and microgranulomas surrounding residual material. Some samples showed minimal abscess formation in the superficial and deep wound bed. No evidence of necrosis, edema or seroma formation, or mineralization was found in any treatment group. The observations were consistent with previous biocompatibility findings and indicate that the preparation hydrogel is biocompatible in a tissue injury model.

### Example 6: In Vivo Safety of the Peptide Hydrogels During Incisional would Healing

Safety and biocompatibility of the peptide hydrogel during cutaneous wound healing were evaluated in vivo in a porcine model of full-thickness incisional wounds. Intradermal application of the peptide hydrogel into full-thickness incisional wounds infected with methicillin-resistant Staphylococcus aureus (MRSA) did not elicit toxicity or inflammation. Treatment with the peptide hydrogel did not impair any aspect of wound healing by gross observation and/or histopathology at any of the studied time points, when compared to both untreated control and comparator product control. Administration of the peptide hydrogel demonstrated superior wound healing performance by reducing swelling and scarring when compared to both untreated control and comparator product.

The peptide hydrogels were successfully manufactured to specifications, with endotoxin levels <0.05 EU/ml.

Material safety and biocompatibility were first evaluated macroscopically by gross observation of the pig full-thickness incisional wounds over time. Wounds were photographed at different time-points (days 3, 7, 14, 21, and 25 for dorsal wounds; days 3, 14, 21, and 25 for neck wounds) and visually scored for pus/drainage, swelling, redness, scarring, and closure. Wound scoring was performed in a blinded fashion by an independent operator. Further assessment was performed by histology. Wound tissue was harvested at different time-points (days 3, 7, and 21 for pig dorsal wounds, and days 25 for neck wounds), and processed for histological analysis. Mechanical testing was performed to evaluate wound tensile strength at day 25 post-incision.

To assess safety and biocompatibility of the hydrogel in vivo, 2 cm full-thickness incisional dermal wounds were created in the dorsum of a pig. Incisional wounds were inoculated with 10⁴ colony-forming units (CFU) of MRSA and incubated for 15 minutes. Wounds were then either left untreated (Infection Control), treated with intradermal peptide hydorgel (test article), or treated with intradermal gentamicin collagen sponge as comparator product (GentaColl®, Resorba Medical GmbH, Nurnberg, Germany) for 10 minutes prior to closing with VICRYL® sutures and covering with Tegaderm™ transparent wound dressing. Wound tissues were harvested at days 3, 7, and 21 post-incision. Dressings were changed on day 14.

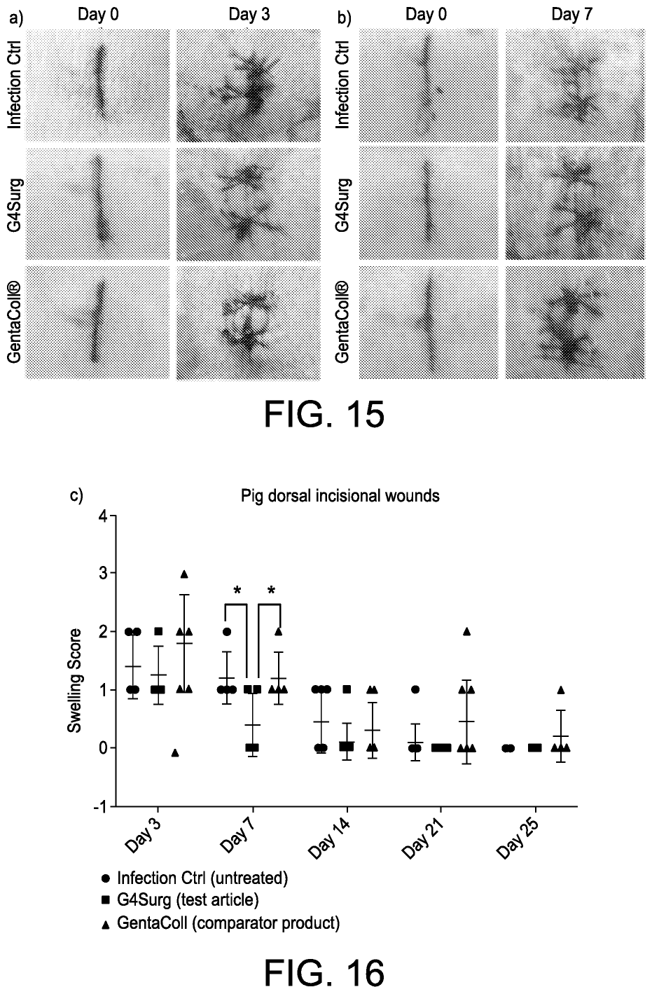

No pus or drainage was evident in any of the pig dorsal wounds at any of the studied time-points. Administration of the peptide hydrogel significantly reduced swelling score (by visual observation) at day 7 post-incision when compared to both untreated infection control and GentaColl®-treated dorsal wounds. Although the same tendency was observed at all other studied time-points, no statistical significance was achieved except at day 7 (as shown in the images of FIG. 15). Wound redness was minimal irrespective of treatment and no differences were observed between treatment groups at any of the studied time-points. As expected, no scarring was evident at day 3 or day 7 post-incision, irrespective of treatment group. However, wound swelling appeared to be more pronounced in GentaColl®-treated wounds at days 3 and 7.

The swelling score data is presented in the graph of FIG. 16. Briefly, FIG. 16 is a graphical representation of wound swelling score at days 3, 7, 14, 21, and 25. Wound swelling was scored as follows: 0=absent/not evident; 1=possibly present/minimal; 2=mild-moderate; 3=severe. The peptide hydrogel intradermal application resulted in a statistically significant reduction of wound swelling score at day 7 compared to both untreated control and comparator product. A slight, non-significant reduction was observed in the peptide hydrogel-treated wounds at all other studied time-points when compared to GentaColl®, and up to day 21 when compared to untreated Infection Control. Data presented as individual points, line shows mean and error bars±SD. Comparisons by two-way ANOVA. * p<0.05.

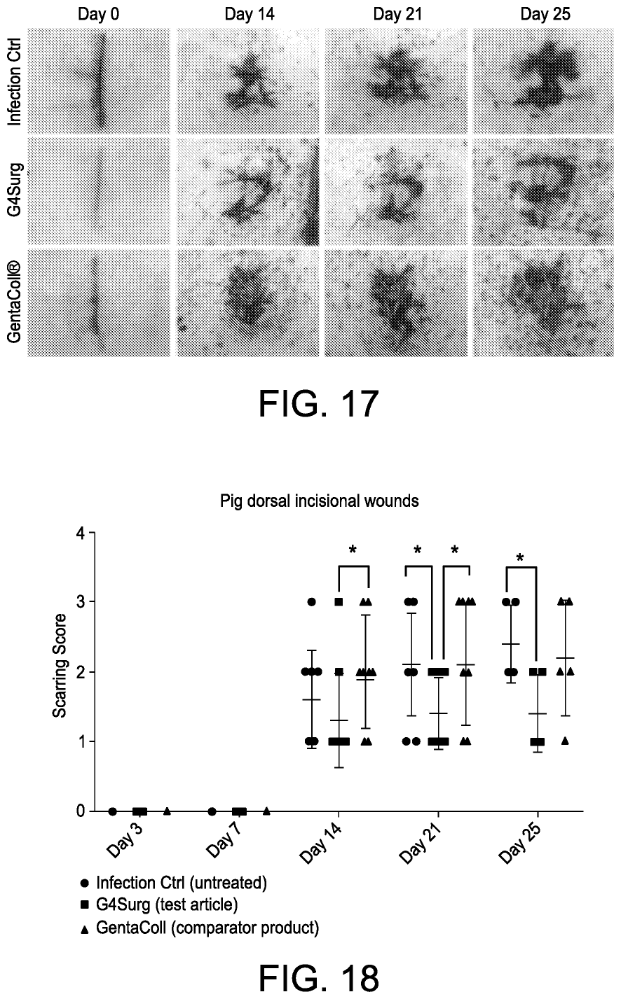

The peptide hydrogel-treated dorsal wounds showed reduced scarring score at day 14 compared to GentaColl®-treated wounds, reduced scarring score at day 21 compared to both infection control and GentaColl®-treated wounds, and reduced scarring score at day 25 compared to infection control wounds (as shown in the images of FIG. 17). At day 7 post-incision, complete wound closure was apparent in all dorsal wounds irrespective of treatment, suggesting that neither of the materials delayed re-epithelialization. The peptide hydrogel-treated wounds appeared to have smaller and thinner scars compared to infection control and GentaColl®-treated wounds.

The scarring score data is presented in the graph of FIG. 18. Briefly, FIG. 18 is a graphical representation of wound scarring score at days 3, 7, 14, 21, and 25. Wound scarring was scored as follows: 0=absent/not evident; 1=present at/around sutures only; 2=present at/around sutures and incision line; 3=present at/around sutures, incision line, and beyond. The peptide hydrogel intradermal application showed a significant reduction in scarring score at day 14 compared to comparator product (GentaColl®), at day 21 compared to both untreated infection control and GentaColl®, and at day 25 compared to infection control. Data presented as individual points, line shows mean and error bars±SD. Comparisons by two-way ANOVA. * p<0.05.

In vivo safety of the peptide hydrogel during incisional wound healing (porcine model) was further assessed by histopathological analysis of the wound tissues at days 3, 7, and 21 post-incision for dorsal incisional wounds. Both materials (peptide hydrogel, test article and GentaColl®, comparator product) were well tolerated and did not show signs of increased inflammation throughout the implantation period, i.e., up to 25 days post-incision. Residual material was present in all GentaColl®-treated wounds at day 3 and day 7 post-incision, resulting in incomplete apposition of the wound edges and a slight, non-significant, reduction in fibrosis at day 7. However, by day 21 no residual GentaColl® was observed. Histological findings revealed that GentaColl® application resulted in a slight, non-significant increase in tissue fibrosis 21 days post-incision. No residual material was observed in any of the peptide hydrogel-treated wounds at any of the studied time points. No signs of tissue necrosis were present in any of the wounds at any of the studied time points irrespective of treatment, suggesting that neither of the materials exhibits toxicity. By day 7 post-incision, all wounds appeared to be closed, irrespective of treatment.

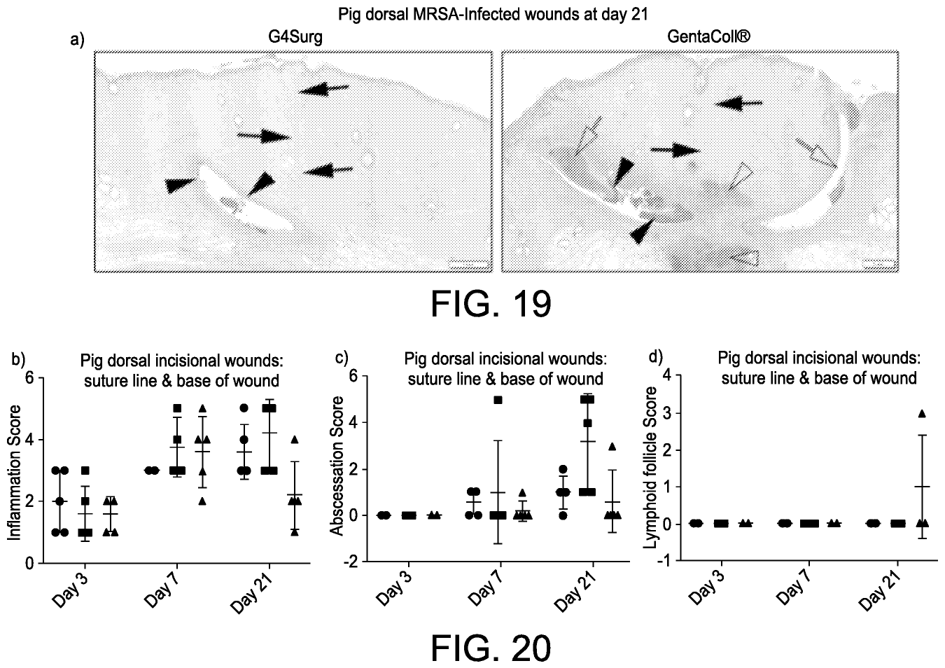

Histological analysis revealed only minimal inflammatory response at the incision line and superficial dermal aspect of the wounds at all studied time-points, irrespective of treatment, suggesting that neither of the materials is eliciting acute or chronic inflammation. FIG. 19 includes H&E images of day 21 pig dorsal wounds. The images show reduced inflammatory cell infiltration at the incision line in peptide hydrogel-treated wounds. The incision line identified by black arrows is barely visible with minimal inflammation and the suture line identified with black arrowheads shows some abscessation. The incision line is evident in the GentaColl®-treated wounds (black arrows). Some abscessation (black arrowheads) is present along the suture lines (open arrows), and lymphoid follicle development is evident (open arrowheads).

Increased overall inflammation and abscess formation were found in the suture line and base of wounds that received the peptide hydrogel compared to GentaColl® at day 21 post-wounding. Abscessation was only present at the suture line and never along the incision line. Lymphoid follicles were evident in the deep aspect of 2 of 5 GentaColl®-treated wounds but none was found in any of the peptide hydrogel-treated wounds. These results are summarized in the graphs of FIG. 20. Briefly, graph (B) of FIG. 20 shows overall inflammation score along the suture line and base of wound. Only minimal inflammation was noted in the incision line and superficial aspect of the pig dorsal wounds, irrespective of treatment. However, in the suture line and deep aspect of wounds, inflammation was more pronounced across treatments. Graph (C) of FIG. 20 shows abscessation score along suture line and deep aspect of wounds. Abscess formation was not found in any of the wounds along the incision line and superficial dermal aspect of wounds. Along the suture line, some abscessation was present in all groups at days 7 and 21 post-incision. At day 21, abscessation at the suture line was increased in peptide hydrogel-treated wounds. Graph (D) of FIG. 20 shows lymphoid follicle score along suture line and base of wounds. Data presented as individual points, line shows mean and error bars±SD. Comparisons by two-way ANOVA. *p<0.05.

No differences in giant body cell or macrophage infiltration were observed between treatment groups. No multinucleated giant cells were found in any of the wounds at any of the studied time-points, irrespective of treatment. Minimal macrophage infiltration (1-5 cells/high power field) was observed in day 7 dorsal wounds, across all treatment groups. In addition, no significant differences were noted between groups in foreign body inflammation score. The peptide hydrogel application resulted in a slight, non-significant increase in foreign body inflammation score in day 7 wounds. However, at day 21 post-incision, wounds that received the peptide hydrogel presented a non-significant reduction (rather than increase) in foreign body inflammation score.

As expected, no fibrosis was observed at day 3 post-incision. At days 7 and 21, minimal to mild fibrosis was observed across treatment groups. A slight, non-significant reduction in fibrosis score was noted in day 7 wounds treated with GentaColl®, likely due to the presence of residual material that prevented complete apposition of the wound edges. However, at day 21 no residual material was found and a non-significant increase (rather than reduction) in fibrosis score was observed in GentaColl®-treated wounds. This is in agreement with the visual observations showing that day 21 wounds treated with GentaColl® presented a higher scarring score when compared to hydrogel-treated wounds. Histological findings also confirmed complete re-epithelialization in all pig dorsal wounds at day 7 post-incision.

Mechanical testing of day 25 pig dorsal wound samples was also performed using a tensile testing system (Instron®, Norwood, MA). The tests were conducted at room temperature, with a load cell of 2 kN and a strain rate of 10 mm/min for 2 hours. No differences in tensile strength were observed between treatment groups. However, day 25 wound tissue samples across all groups presented lower tensile strength compared to intact (unwounded) skin. Similarly, Young's Modulus calculations, and indicator of tissue stiffness, revealed no differences between treatment groups and significant differences between intact skin and all wound treatment groups.

The mechanical testing data confirms that the peptide hydrogel does not have a negative impact in wound healing, since the tissue tensile strength and tissue stiffness were almost the same in untreated infection control, hydrogel-treated, and GentaColl® (comparator product)-treated samples.

Additional full-thickness incisional dermal neck wounds were created and inoculated with MRSA as described above, treated with test article (peptide preparation) or left untreated (Infection Control), and harvested 25 days post-incision. No pus or drainage was evident in any of the pig neck wounds at the recorded time-points. The peptide hydrogel-treated wound showed a slight, non-significant reduction in swelling score at day 3 post-incision when compared to infection control wounds by visual evaluation. Wound redness was absent or minimal across treatment groups and no differences were observed at any of the studied time-points. Hydrogel-treated neck wounds showed reduced scarring score by gross observation at day 14 compared to infection control wounds. Histological data of day 25 neck wounds showed no differences in overall inflammation or foreign body inflammation. A slight, non-significant reduction in fibrosis score was noted in the hydrogel-treated wounds compared to infection control wounds.

The data indicates that the hydrogel is non-toxic and non-inflammatory during incisional wound healing. Moreover, application of the peptide preparation into incisional wounds showed superior tissue regenerative performance compared to comparator product by reducing wound swelling and scarring. The findings demonstrate that the peptide hydrogel is safe and biocompatible for application into dermal full-thickness incisional wounds.

### Example 7: Therapeutic In Vivo Efficacy of the Peptide Hydrogels to Prevent Infection

Therapeutic efficacy of the peptide hydrogel in preventing infection was evaluated in vivo in a rat model of surgical site infection. Intradermal application of the peptide hydrogel into abdominal wounds infected with MRSA resulted in significantly lower infection scores compared to untreated control by visual inspection of the wounds. No statistical significance was achieved, However, a tendency towards reduction in bacterial load was observed.

A rat animal model of surgical site infection was used for the study. A bilateral full-thickness 3 cm vertical abdominal wall (skin and muscle) incision was created in each animal. The inner half layer of abdominal wall (peritoneum, transversus muscle) was sutured, and inoculated with MRSA. Bacterial inoculum concentrations in a defined volume were introduced in the open/unclosed layers of abdominal wall incision and incubated for 15 minutes. The test article was applied and incubated for 10 minutes before totally closing abdominal wall with suture. An additional dose of test article was applied at the closed wound site. Tissue adhesive was used to affix Tegaderm™ to the skin in a radius of approximately 2 cm around the wounds. An elastic bandage was used to provide an additional barrier, and e-collars were placed to prevent chewing on the bandages by the rats. Approximately 24 hours post-incision, animals were euthanized. Wounds were photographed and visually scored, and tissue samples were harvested for microbiological analysis.

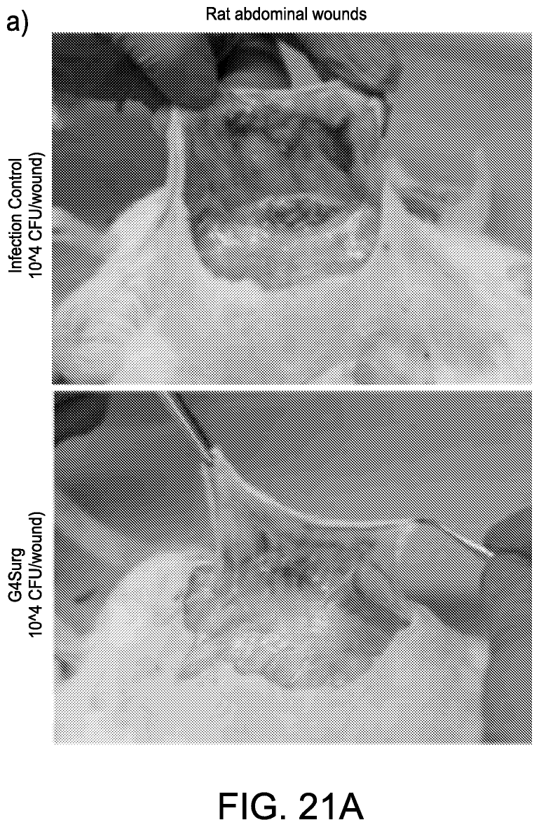

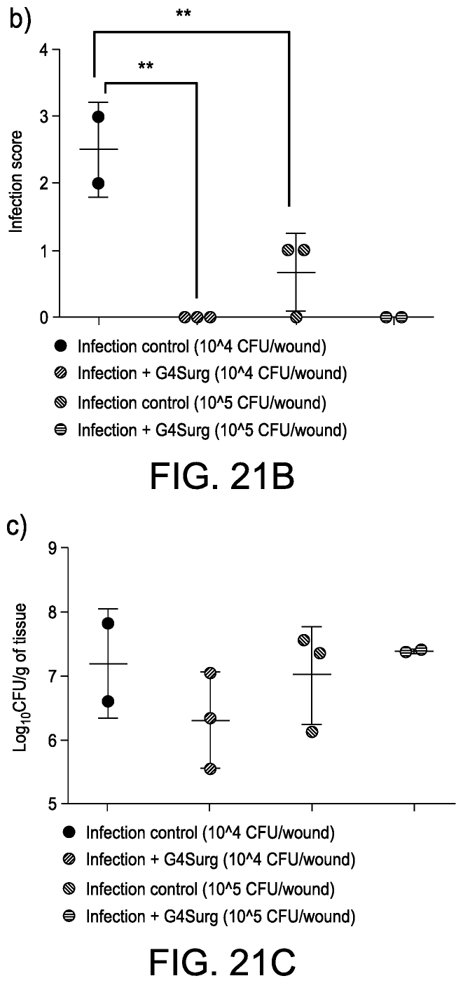

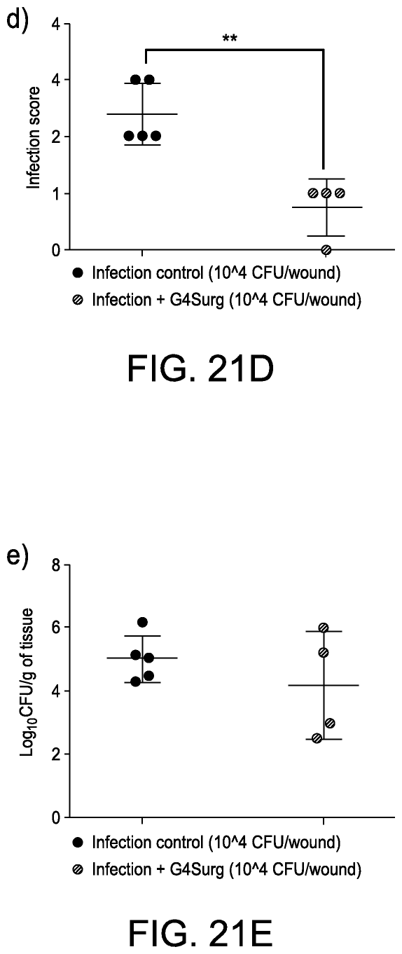

In the first study, two different concentrations of bacterial inoculum (10⁴ CFU and 10⁵ CFU of MRSA) were tested. Application of the peptide hydrogel resulted in a significant lower infection score (by visual evaluation of gross wounds) compared to infection control in rat abdominal wounds that received either 10⁴ CFU or 10⁵ CFU of MRSA, as shown in the images of FIG. 21A. Briefly, FIG. 21A includes photographs of rat abdominal wounds at necropsy (approximately 24 hours post-incision), untreated (top panel) and treated with the peptide hydrogel (bottom panel). Evident signs of infection (pus, swelling, and redness) are evident in untreated infection control wounds.

The data is presented in FIGS. 21B-21E. Wounds were visually scored for infection as follows: 0=infection absent/not evident; 1=minimal-mild infection; 2=moderate infection; 3=severe infection. Infection score in rat abdominal wounds inoculated with two different concentrations (10⁴ or 10⁵ CFU) of MRSA (FIG. 21B), or with a single dose (10⁴ CFU) of inoculum (FIG. 21D). Respective microbiology data presented as log 10 CFU/g of tissue for two different concentrations (10⁴ or 10⁵ CFU) of MRSA (FIG. 21C), or with a single dose (10⁴ CFU) of inoculum (FIG. 21E). Data presented as individual points, line shows mean and error bars±SD. Comparisons by one-way ANOVA, or unpaired student's t-test. ** p<0.01.

There was a tendency for bioburden reduction in the hydrogel-treated wounds, even if no statistical significance was achieved (FIG. 21C). When comparing infection control wounds inoculated with 10⁴ CFU of MRSA to infection control wounds that received 10⁵ CFU, infection score was lower at the higher concentration of inoculum and no significant differences were observed in bacterial counts at the end of the study (day 1 post-incision). In the second study, abdominal wounds were inoculated with a single dose of MRSA, 10⁴ CFU. Visual observation of gross wounds confirmed a significant reduction in infection score by the hydrogel preparation (FIG. 21D), even though microbiology findings did not correlate with visual scores (FIG. 21E).

Additional wounds were created in the dorsum of the rats. A total of four bilateral full-thickness 1.5 cm vertical (skin and muscle) incisions were made. Wounds were inoculated with 10⁴ CFU of MRSA, treated with test article (peptide hydrogel) and comparator product (GentaColl®), sutured, dressed, and harvested at necropsy (approximately 24 hours post-incision).

Rat dorsal wounds treated with the peptide hydrogel or GentaColl® presented significantly reduced infection scores when compared to untreated infection control wounds. Peptide hydrogel-treated and GentaColl®-treated wounds had the same infection score, suggesting similar therapeutic efficacy in preventing infection. GentaColl®-treated wounds showed a significant reduction in bacterial counts. More studies are needed to determine the peptide hydrogel efficacy on reducing wound bioburden.

The pig dorsal wounds inoculated with 10⁴ CFU of MRSA (as described in Example 6) were also processed for microbiological analysis. Pig dorsal wounds treated with the peptide hydrogel also showed a tendency for reduction in bacterial load compared to infection control wounds at all studied time-points, even if no statistical significance was reached. GentaColl® application significantly reduced bioburden in day 3 wounds when compared to infection control wounds. However, no differences were observed at the remaining time-points. By day 21, the bacterial counts were virtually the same in GentaColl®-treated and untreated wounds.

The peptide hydrogel significantly reduced infection score by visual inspection in all studied wound models, even if it failed to significantly reduce CFU/g of tissue by microbiological analysis. The tissue samples used for microbiological analysis included the suture lines, where local signs of infection such as abscessation were present, and the different mechanisms of action, e.g., leaching in case of GentaColl® and non-leaching in case of the peptide hydrogel, have likely contributed to the contradictory effects in the incision line as compared to the suture line. Future studies, designed to distinguish between the effect of treatments and the effect of sutures, are needed to draw proper conclusions. For example, the use of triclosan-coated sutures or surgical staples impregnated in antibiotic instead of regular, uncoated sutures, could help address this issue.

### Example 8: Treatment of Full-Thickness Wound Infected with MRSA

Effectivity of the hydrogel at 0.75% w/v and 1.5% w/v of the peptide were tested in the treatment of full-thickness wounds infected with MRSA.

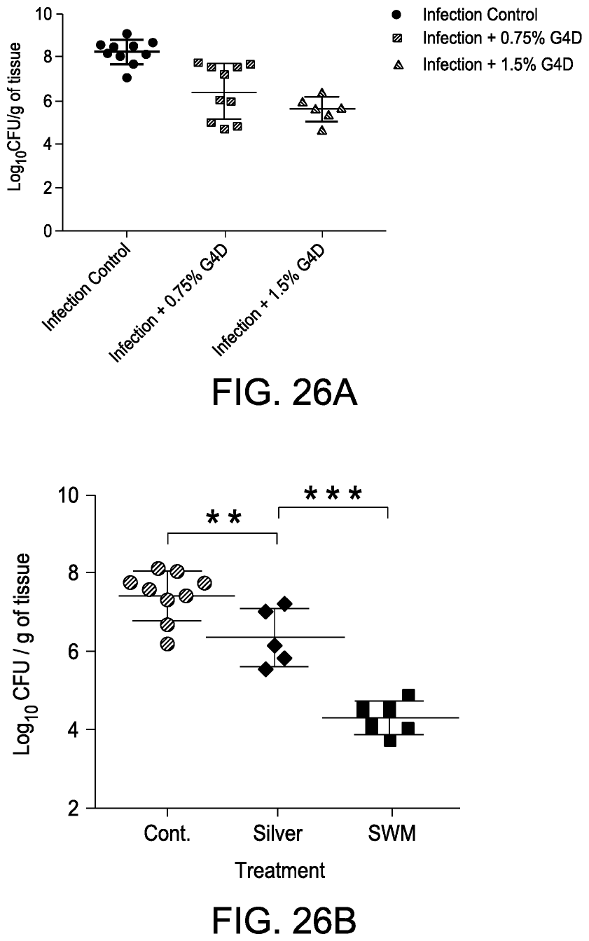

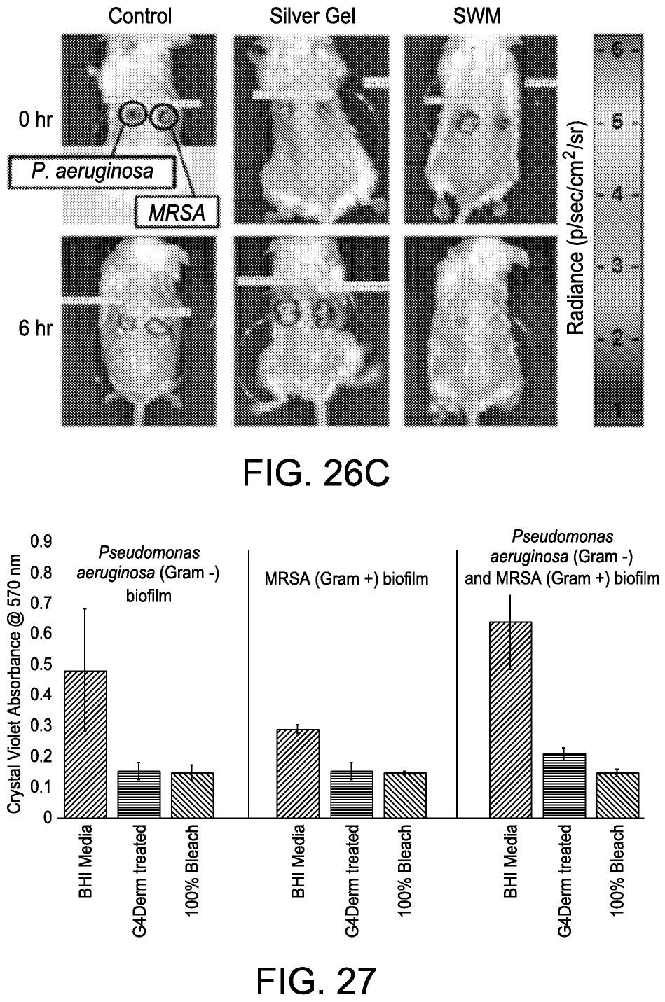

Briefly, full-thickness incisions were created in mice as previously described. The incisions were infected with MRSA microbial colony. The infected wounds were treated with control, silver gel, 0.75% w/v peptide hydrogel (denoted SWM), or 1.5% w/v peptide hydrogel. MRSA proliferation was measured at 24 hours post-infection. The results are presented in the graphs and images of FIGS. 26A-26C.

As shown in the graph of FIG. 26A, treatment with both 0.75% w/v and 1.5% w/v peptide preparations reduced microbial proliferation, as compared to the control. Furthermore, there was no significant difference between treatment with 0.75% w/v peptide preparation and treatment with 1.5% w/v peptide preparation.

As shown in the graph of FIG. 26B, treatment with 0.75% w/v peptide preparation (SWM) reduced microbial proliferation, as compared to both silver gel and the control. A 3.5 log 10 CFU reduction of MRSA was shown within 24 h of a single application of SWM. As shown in the images of FIG. 26C, MRSA and P. aeruginosa were both reduced within 6 hours of a single application of SWM. The reduction was greater than both silver gel and control.

### Example 9: Reduced Bioburden in Full-Thickness Wounds in a Mouse Model

Effectivity of the hydrogel was tested against bioluminescent MRSA and P. aeruginosa. Regeneration is promoted in chronic wounds by eliminating pathogens and regenerating tissue. The preparations disclosed herein exhibit inherent broad-spectrum antimicrobial activity which prevent biofilm formation and eradicate established biofilms, provide cell attachment sites as a tissue scaffold, and have mechanical properties which provide ease of application and conformal void filling.

Briefly, full-thickness incisions were created in mice as previously described. The wounds were inoculated with bacteria. The wounds were treated with the peptide hydrogel, or Germ Shield™ silver gel (Curad, Northfield, IL) after 30 minutes.

Treatment was qualitatively examined. The peptide hydrogel essentially eliminated the bioluminescent bacteria.

In a quantitative assessment, treatment with the hydrogel resulted in 3-log reduction in MRSA after 24 hours compared to untreated controls. Additionally, the peptide hydrogel significantly reduced wound bioburden resulting from all inoculations (10⁴-10⁶ CFU/wound). The results are shown in the graph of FIG. 27.

Accordingly, a single application of the preparation to MRSA-colonized wounds reduces bioburden in 24 hours.

### Example 10: In Vitro Biofilm Treatment Study

Anti-biofilm activity of the hydrogel was investigated. The hydrogel successfully eliminated 100% of P. aeruginosa PA01 and prevented biofilm formation in vitro.

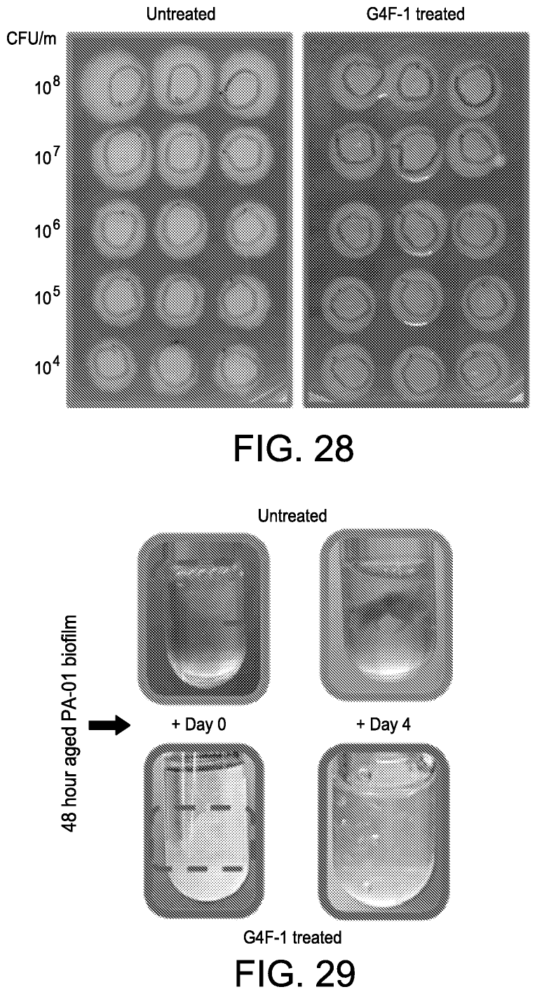

Briefly, planktonic PA01 was spotted on agar plates at concentrations of 10⁴ to 10⁸ CFU/ml. Thirty minutes later, the preparation was added to the plates in amounts of 10 μl/spot. After 24 hours, the PA01 colonies were quantified. Images of the plates are shown in FIG. 28.

The results show that the hydrogel was effective in eliminating 100% of PA01. Thus, administration of the preparation prevented biofilm formation in vitro.

Additionally, 48-hour aged PA01 biofilms were prepared from initial inoculations of 10⁶ CFU/ml. The aged biofilms were treated with the preparation. At day 4 post-treatment the treated biofilms were clear, as compared to untreated controls which matured into brown slime. Images of the biofilms are shown in FIG. 29.

The results show that the hydrogel was effective in inhibiting biofilm growth in vitro.

### Example 11: Ex Vivo Biofilm Treatment Study

Ex vivo studies were performed on pig skin biofilms to test treatment of MRSA and P. aeruginosa PA01.

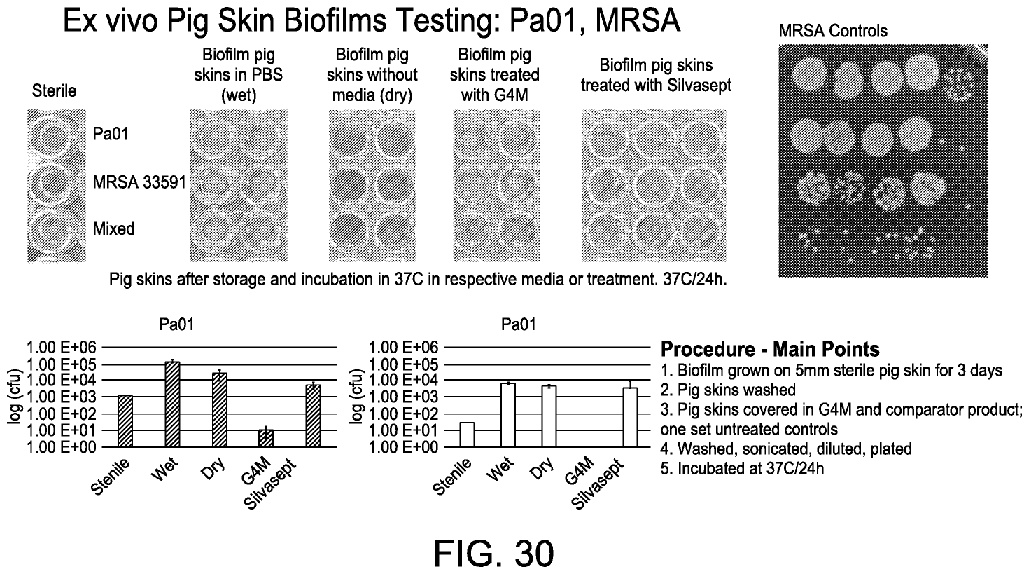

Briefly, biofilms were grown on 5 mm sterile pig skin for 3 days. The pig skins were washed and covered in the hydrogel or comparator product. The treated pig skins were washed, sonicated, diluted, and plated. The treated pig skins then incubated at 37° C. for 24 hours before the colonies were quantified. The results and images are shown in FIG. 30.

As shown in FIG. 30, the peptide hydrogel (G4M) showed a significant decrease in both PA01 and MRSA bacterial colonies after treatment.

### Example 12: Cell Viability Assay

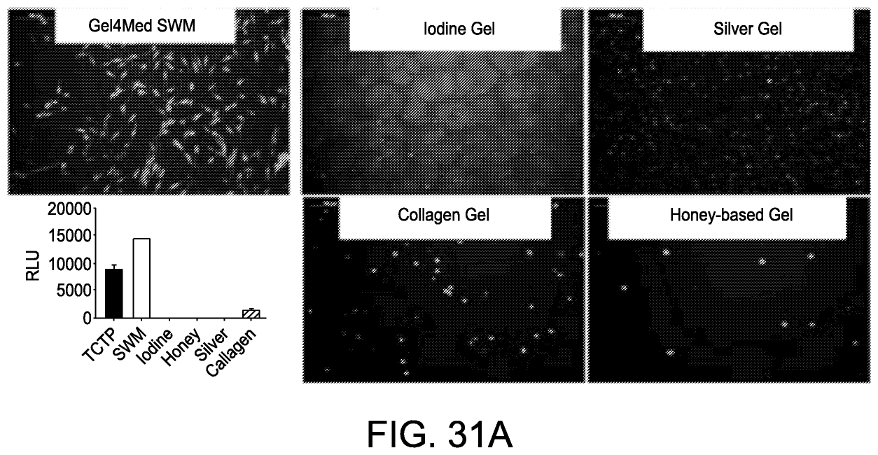

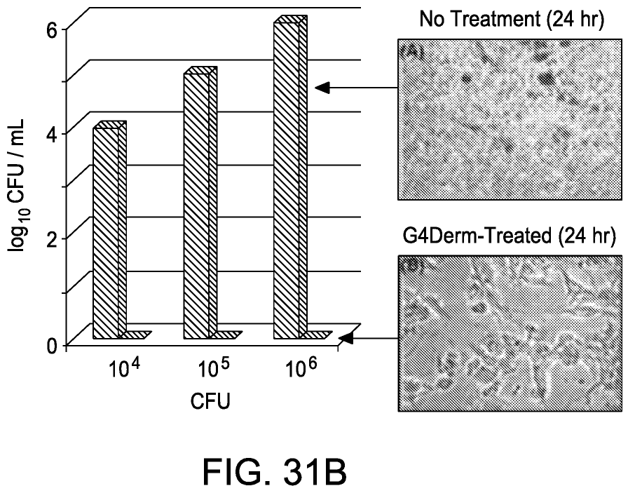

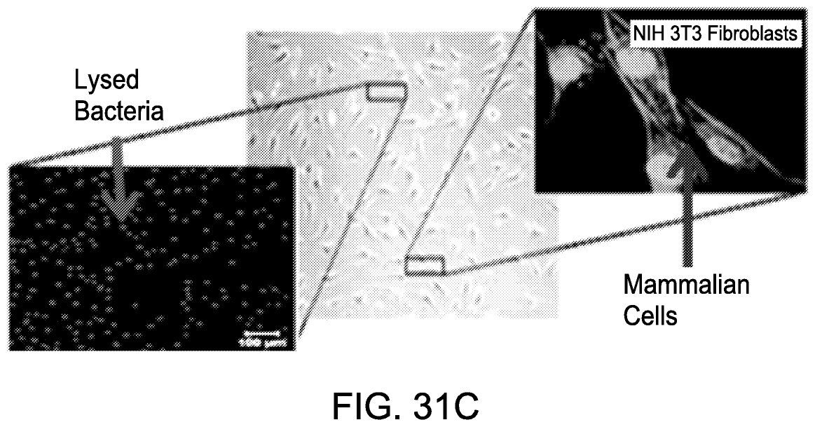

Host cell and tissue compatibility is an important feature to promote wound closure and tissue regeneration. The peptide hydrogels are cytocompatible with a variety of mammalian cell types, including erythrocytes and macrophages. When compared with current categories of antimicrobials and commercially available gels (iodine gel, silver gel, collagen gel, and honey-based gel), the 0.75% w/v peptide hydrogel (SWM) demonstrated significantly superior cytocompatibility with fibroblasts (cell line CRL-12424) seeded onto the gels at 24 hours of culturing across all tested groups (p<0.001) in the form of higher cell viability, cell spreading, and cell attachment (FIG. 31A). In the graph of FIG. 31A, cell viability is expressed as relative fluorescent units (RLU). Additionally, microscopy images indicate cell spreading in the hydrogel, whereas minimal to no cells attached to comparator antimicrobial gels.

Simultaneous seeding of MRSA with mammalian cells (cell line CRL-12424) onto the hydrogel showed that bacteria were killed while mammalian cells remained >90% viable after 24 hours. The results are shown in the graph of FIG. 31B. The viable mammalian cells also showed the ability to proliferate, exhibited fibroblast cell spreading, focal adhesions, and 3-D proliferation. Simultaneous seeding of E. coli with mammalian fibroblasts (cell line NIH-3T3) onto the hydrogel shown in the images of FIG. 31C further shows selective toxicity of the hydrogel against bacteria and rescue of mammalian cells. Thus, the peptide hydrogel exhibits specific toxicity towards pathogens while remaining compatible with mammalian cells.

### Example 13: Antimicrobial Efficacy of the Peptide Hydrogel

Antimicrobial efficacy of the 0.75% w/v peptide hydrogel (SWM) and the 1.5% w/v peptide hydrogel (SWM Extra) were tested. After 24 hours, complete elimination of bacterial colonies was observed, indicating that preparation exhibits excellent antimicrobial efficacy.

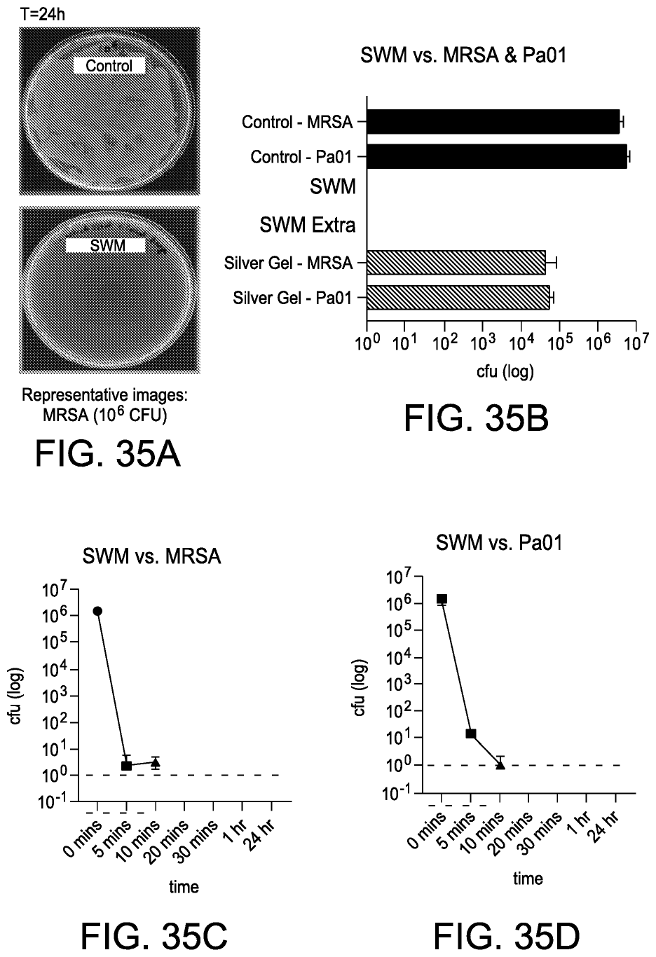

A 10 μl volume of gram-positive MRSA or gram-negative P. aeruginosa PA01 (10⁴-10⁶ CFU) was plated over the preparation and incubated at 37° C. for 24 hours. FIG. 35A is an image of the MRSA culture plated over SWM hydrogel or control. The results are shown in the graphs of FIGS. 35B-35D.

After 24 hours, the plates were imaged, and the resulting colonies were enumerated. As shown in the graph of FIG. 35B, no colonies were observed on the SWM or SWM Extra treated samples. However, colonies were observed in the control and silver gel treated samples. Thus, complete (100%) elimination of bacteria was achieved. Furthermore, as shown in the graphs of FIGS. 35C-35D, very few, if any, colonies were observed on the SWM treated samples after 10 minutes and full clearance was observed within 20 minutes, for both MRSA and P. aeruginosa colonies. Accordingly, rapid elimination of bacteria is achieved with administration of the peptide hydrogel.

### Example 14: Gram-Positive and Gram-Negative Antimicrobial Efficacy of the Peptide Hydrogel

Antimicrobial efficacy of the 0.75% w/v peptide hydrogel (SWM) was tested. After 24 hours, complete elimination of various gram-positive and gram-negative bacterial colonies was observed, indicating that preparation exhibits broad-spectrum antimicrobial efficacy.

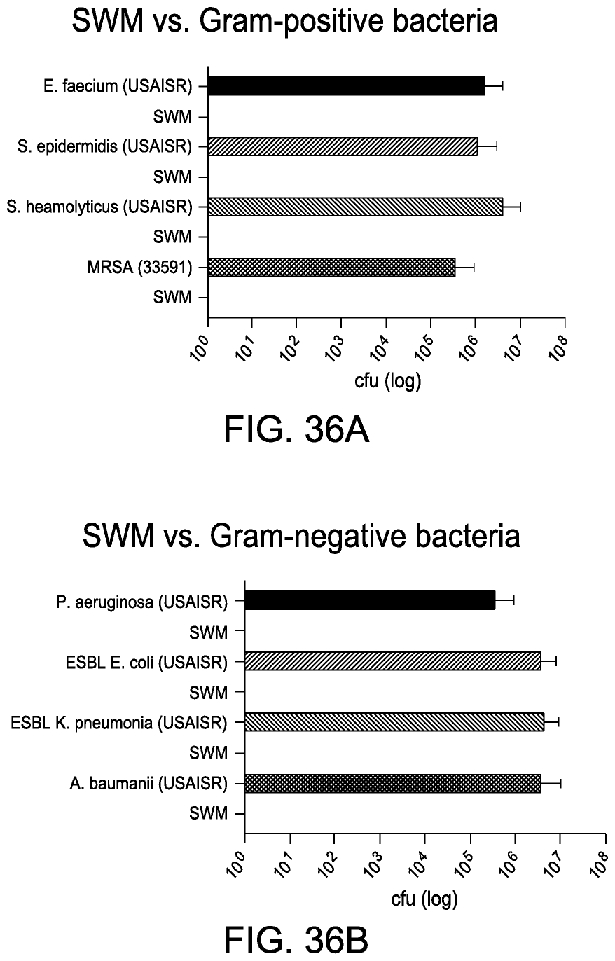

Gram-positive colonies of E. faecium, S. epidermidis, S. heamolyticus, and MRSA were each plated over the preparation and incubated at 37° C. for 24 hours. Gram-negative colonies of P. aeruginosa, ESBL E. coli, ESBL K. pneumonia, and A. baumanii were each plated over the preparation and incubated at 37° C. for 24 hours. The results are shown in the graphs of FIGS. 36A-36B.

After 24 hours the colonies were enumerated. As shown in the graph of FIG. 36A, no colonies were observed on the SWM treated gram-positive bacteria samples. As shown in the graph of FIG. 36B, no colonies were observed on the SWM treated gram-negative bacteria samples. Accordingly, complete (100%) elimination of several tested gram-positive and gram-negative bacterial colonies was achieved. The peptide hydrogel preparations disclosed herein show broad spectrum antimicrobial efficacy.

### Example 15: Antimicrobial Efficacy Against Monomicrobial and Polymicrobial Biofilms

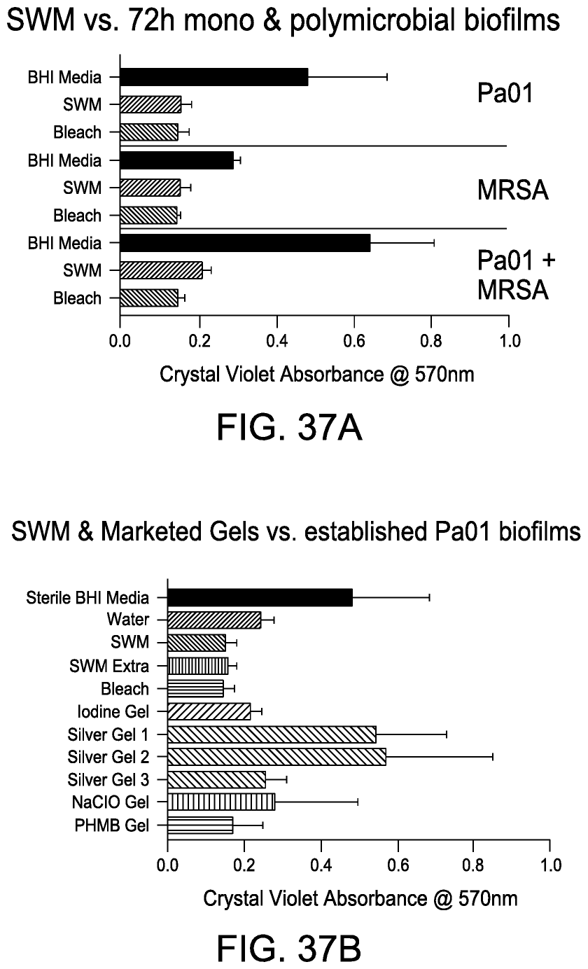

Antimicrobial efficacy of the 0.75% w/v peptide hydrogel (SWM) against monomicrobial and polymicrobial biofilms was tested. The preparation was tested against MRSA, P. aeruginosa (PaO1), and mixed MRSA and P. aeruginosa biofilm colonies. After 72 hours, crystal violet absorbance at 570 nm was measured to quantify the biofilm colony. The results are shown in the graph of FIG. 37A. As shown in the graph of FIG. 37A, the hydrogel preparation exhibits excellent efficacy against monomicrobial and polymicrobial biofilms comparable to treatment with 100% bleach.

Antimicrobial efficacy of the 0.75% w/v peptide hydrogel (SWM) and the 1.5% w/v peptide hydrogel (SWM Extra) against P. aeruginosa (PaO1) was tested and compared to commercially available treatments bleach, iodine gel, three formulations of silver gel, NaClO gel, and polyhexamethylene biguanide (PHMB) gel. Crystal violet absorbance at 570 nm was measured to quantify the biofilm colony. The results are shown in the graph of FIG. 37B. As shown in the graph of FIG. 37B, both formulations of the hydrogel preparation exhibited excellent efficacy against P. aeruginosa biofilms comparable to commercially available antimicrobial treatments.

### Example 16: Prophetic Treatment of Complex Anal Fistulas

The peptide hydrogel preparations disclosed herein may be administered for treatment of anal fistulas. The proposed treatment will be a sphincter sparing non-invasive first-line treatment for treating complex anal fistula. There is a need for an effective treatment for complex anal fistulas to provide successful and predictable wound healing outcomes.

Anal fistula is a small tunnel that connects an abscess, an infected cavity in the anus, to an opening on the skin around the anus. Ninety percent of fistulas are a chronic outcome of cryptoglandular infection and ten percent of fistulas (classified as secondary) are a result of inflammatory bowel disease (IBD), neoplasm, radiation, or trauma. Fistulas may be classified into simple or complex based on their etiology and comorbidities. Generally, complex anal fistulas may be categorized as having a track that crosses >30% to 50% of the external sphincter, multiple tracks with multiple external and internal openings, recurrent episodes or symptoms (e.g., chronic fistulas), or the patient has preexisting incontinence, local irradiation, or Crohn's disease. Thus, complex anal fistulas that may be treated by the methods disclosed herein may be associated with one or more of these etiologies or comorbidities.

The peptide preparations disclosed herein may be employed to treat simple and complex anal fistulas. However, simple or low fistula-in-ano has a more predictable healing outcome. Therefore, the proposed prophetic study will examine treatment of complex anal fistula. The conventional treatment approach to complex anal fistulas is typically more difficult, with higher rates of failure and functional disability. Conventional treatments include topical application of fibrin glue, collagen paste, and collagen plugs to the anal fistula target site. The main reasons for low and variable healing rates of the conventional treatments include device colonization, device expulsion, degradation, biological variability of sourced materials (matrix and Thrombin crosslinkers), seroma formation, difficulty to use, and variability of surgeon's capabilities. Furthermore, none of the conventional treatments address the potential side effect of intersphincteric tract infection and/or sepsis. For example, biofilm producing bacteria are more prevalent in non-healing chronic fistulas emphasizing the role of unaddressed infection in chronicity of complex fistulas.

Successful outcomes for sphincter sparing in-lumen fistula treatment will retain dignity and quality of life in patients with incontinence risk, reduce morbidity, reduce infections, and prevent recurrence. The sphincter sparing in-lumen fistula treatment disclosed herein will overcome the challenges of biological variability and device colonization, currently faced by conventional void-filling products. In particular, the antimicrobial and biofilm treating properties of the peptide hydrogel have previously been discussed.

The proposed complex anal fistula treatment will include administration of the peptide hydrogel to a target site of an anal fistula by injection. Such mode of administration will inhibit or prevent irreversible damage to the sphincter. An alternate proposed complex anal fistula treatment will include sphincter sparing topical application of the peptide hydrogel to a target site of an anal fistula, optionally with a delivery device. In certain instances, the application may be assisted by an imaging device, such as an endoscope. The methods of administration will be simple, safe, painless and preserve continence. Follow-on administrations can be repeated to increase the healing rate, without compromising following conventional surgical procedures.

The preparations administered may be combined with medical treatments, such as cells and anti-inflammatory agents. Localized delivery of these agents is a promising approach in overcoming any toxicity concerns of the medical treatments, while increasing bioavailability at lower dosing of the agents. Furthermore, localized administration of inflammation agents would be possible, as shown by the drug delivery capacity of the peptide hydrogel.

Thus, the proposed treatment of complex anal fistula will: (i) provide a matrix with wound healing capacity, (ii) have excellent void filling properties, (iii) be easy to apply with repeatable application option, (iv) be resistant against colonization and limit use of extended antibiotics (v) be synergistic with medical treatments and surgery (vi) be sphincter sparing, (vii) be cost effective, and (viii) be extendable to drug delivery for local delivery of therapeutics when second line approaches are needed.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

1. A method of treating a microbial contamination associated with a community of microorganisms of a subject in need thereof, comprising:
administering to a target site of the subject a preparation comprising a purified amphiphilic peptide in an aqueous biocompatible solution, the peptide comprising a folding group having a plurality of charged amino acid residues and hydrophobic amino acid residues arranged in a substantially alternating pattern and a turn sequence, the peptide being configured to self-assemble into a hydrogel, in an amount effective to promote deactivation of the microbial contamination.

2. A method of eliminating or inhibiting proliferation of a target microorganism associated with a community of microorganisms at a target site, comprising:
administering to the target site an effective amount of a preparation comprising a purified amphiphilic peptide in an aqueous biocompatible solution, the peptide comprising a folding group having a plurality of charged amino acid residues and hydrophobic amino acid residues arranged in a substantially alternating pattern and a turn sequence, the peptide being configured to self-assemble into a hydrogel, in an amount effective to eliminate or inhibit proliferation of the target microorganism.

3. The method of claim 1, wherein the target site is a local site of the microbial contamination.

4. The method of claim 1, wherein the peptide comprises an effective amount of counterions, to form the hydrogel, the peptide being free of chloride counterions and the counterions selected from acetate and citrate counterions.

5.-6. (canceled)

7. The method of claim 1, wherein the amount is sufficient to sterilize at least 90% of the target microorganism at the target site.

8. The method of claim 1, wherein the target microorganism is a pathogenic microorganism.

9. The method of claim 8, wherein the target microorganism is a species of a genus selected from Bacillus, Bartonella, Bordetella, Borrelia, Brucella, Campylobacter, Chlamydia, Chlamydophila, Clostridioides, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yersinia.

10. The method of claim 1, wherein the target site is a tissue selected from mesenchymal tissue, connective tissue, muscle tissue, nervous tissue, embryonic tissue, dermal tissue, bone tissue, dental tissue, corneal tissue, cutaneous tissue, integumental tissue, soft tissue, and hard tissue, or a biological fluid selected from tears, mucus, urine, menses, blood, wound exudates, and mixtures thereof.

11. (canceled)

12. The method of claim 2, wherein the target site is a surface selected from selected from a household surface, an industrial surface, a food industry surface, and a healthcare surface.

13. The method of claim 12, wherein the target surface is a medical tool surface, a medical implant surface, or a medical device surface.

14. The method of claim 1, comprising administering the preparation to a target site of a subject topically, enterally, or parenterally.

15.-18. (canceled)

19. The method of claim 1, wherein:
the folding group has a sequence comprising Y[XY]_{N}[T][YX]_{M}Y, where X is 1-3 charged amino acids, Y is 1-3 hydrophobic amino acids, T is 2-8 turn sequence amino acids, and N and M are each independently between 2 and 10;
the hydrophobic amino acid residues are independently selected from glycine, alanine, valine, leucine, isoleucine, proline, phenylalanine, methionine, threonine, tryptophan, and combinations thereof;
the charged amino acid residues are independently selected from arginine, lysine, histidine, and combinations thereof; and
the turn sequence amino acids are independently selected from a D-proline, an L-proline, aspartic acid, threonine, asparagine, and combinations thereof.

20.-30. (canceled)

31. The method of claim 1, wherein the peptide is at least 80% purified.

32.-34. (canceled)

35. The method of claim 1, wherein the peptide includes a functional group having between 3 and 30 amino acids, the functional group being engineered to express a bioactive property.

36.-48. (canceled)

49. The method of claim 1, wherein the preparation comprises between 0-1% 0.5% w/v and 8-0% 3.0% w/v of the peptide.

50.-52. (canceled)

53. The method of claim 1, wherein the peptide is anionic or cationic, the cationic peptide having a net charge of from +2 to +11.

54. The method of claim 53, wherein the peptide has a net charge of from +5 to +9.

55. The method of claim 1, wherein the peptide is lyophilized.

56. The method of claim 1, wherein the preparation is sterile.

57.-60. (canceled)

61. The method of claim 1, further comprising debridement of the target tissue prior to administration of the preparation.

62.-63. (canceled)

64. The method of claim 1, wherein the microbial contamination is a microbial colonization or infection.

65. The method of claim 1, wherein the target site is associated with a wound.

66. The method of claim 65, comprising administering the preparation in an amount effective to treat at least one of partial and full thickness wounds (e.g., pressure sores, leg ulcers, diabetic ulcers), first and second degree burns, tunneled/undermined wounds, surgical wounds (e.g., associated with donor sites/grafts, tissue and cell grafts, Post-Moh's surgery, post laser surgery, podiatric, sound dehiscence), trauma wounds (e.g., abrasions, lacerations, burns, skin tears), gastrointestinal wounds (e.g., anal fistulas, diverticulitis, ulcers), and draining wounds.

67. A method of treating biofilm, comprising:
administering to a target site of the biofilm a thermally-stale preparation comprising a purified amphiphilic peptide in an aqueous biocompatible solution, the peptide comprising a folding group having a plurality of charged amino acid residues and hydrophobic amino acid residues arranged in a substantially alternating pattern and a turn sequence, the peptide being configured to self-assemble into a hydrogel, in an amount effective to promote deactivation of a target microbial organism associated with the biofilm.

68. The method of claim 1, further comprising administering to the target site a buffer configured to induce self-assembly of the hydrogel.

69. The method of claim 68, wherein the buffer comprises between about 10 mM and 150 mM sodium chloride and between about 10 mM and 100 mM Bis-tris propane (BTP).

70. The method of claim 2, further comprising administering to the target site a buffer configured to induce self-assembly of the hydrogel.

71. The method of claim 70, wherein the buffer comprises between about 10 mM and 150 mM sodium chloride and between about 10 mM and 100 mM Bis-tris propane (BTP).

72. The method of claim 67, further comprising administering to the target site a buffer configured to induce self-assembly of the hydrogel.

73. The method of claim 72, wherein the buffer comprises between about 10 mM and 150 mM sodium chloride and between about 10 mM and 100 mM Bis-tris propane (BTP).


SEQUENCE LISTING (23 sequences)

SEQ ID NO: 1 - protein, 20 aa, Artificial Sequence
  Description of Artificial Sequence Synthetic peptide
  MOD_RES (10)..(10): D-amino acid
VKVKVKVKVPPTKVKVKVKV

SEQ ID NO: 2 - protein, 20 aa, Artificial Sequence
  Description of Artificial Sequence Synthetic peptide
  MOD_RES (10)..(10): D-amino acid
VKVRVRVRVPPTRVRVRVKV

SEQ ID NO: 3 - protein, 26 aa, Artificial Sequence
  Description of Artificial Sequence Synthetic peptide
  MOD_RES (10)..(10): D-amino acid
VKVRVRVRVPPTRVRVRVKVGRGDSP

SEQ ID NO: 4 - protein, 20 aa, Artificial Sequence
  Description of Artificial Sequence Synthetic peptide
  MOD_RES (10)..(10): D-amino acid
VRVRVKVRVPPTRVEVRVRV

SEQ ID NO: 5 - protein, 20 aa, Artificial Sequence
  Description of Artificial Sequence Synthetic peptide
  MOD_RES (10)..(10): D-amino acid
VRVRVRVKVPPTKVRVRVRV

SEQ ID NO: 6 - protein, 24 aa, Artificial Sequence
  Description of Artificial Sequence Synthetic peptide
  MOD_RES (14)..(14): D-amino acid
RGDSVRVRVRVKVPPTKVRVRVRV

SEQ ID NO: 7 - protein, 36 aa, Artificial Sequence
  Description of Artificial Sequence Synthetic polypeptide
  MOD_RES (10)..(10): D-amino acid
VKVRVRVRVPPTRVRVRVKVGKLTWQELYQLKYKGI

SEQ ID NO: 8 - protein, 31 aa, Artificial Sequence
  Description of Artificial Sequence Synthetic polypeptide
  MOD_RES (10)..(10): D-amino acid
VKVRVRVRVPPTRVRVRVKVRPKPQQFFGLM

SEQ ID NO: 9 - protein, 17 aa, Artificial Sequence
  Description of Artificial Sequence Synthetic peptide
VKVRVRVRVRVRVRVKV

SEQ ID NO: 10 - protein, 20 aa, Artificial Sequence
  Description of Artificial Sequence Synthetic peptide
  MOD_RES (10)..(10): D-amino acid
VKVKVRVKVPPTKVKVRVKV

SEQ ID NO: 11 - protein, 20 aa, Artificial Sequence
  Description of Artificial Sequence Synthetic peptide
VKVRVRVRYNGTRVRVRVKV

SEQ ID NO: 12 - protein, 28 aa, Artificial Sequence
  Description of Artificial Sequence Synthetic peptide
  MOD_RES (18)..(18): D-amino acid
LKKTETQGVKVRVRVRVPPTRVRVRVKV

SEQ ID NO: 13 - protein, 25 aa, Artificial Sequence
  Description of Artificial Sequence Synthetic peptide
  MOD_RES (15)..(15): D-amino acid
SDKPGVLVRVRVRVPPTRVRVRVKV

SEQ ID NO: 14 - protein, 24 aa, Artificial Sequence
  Description of Artificial Sequence Synthetic peptide
  MOD_RES (14)..(14): D-amino acid
RGDSVRVRVRVKVPPTKVRVRVRV

SEQ ID NO: 15 - protein, 20 aa, Artificial Sequence
  Description of Artificial Sequence Synthetic peptide
  MOD_RES (10)..(10): D-amino acid
VKVKVKVKVPPTKVEVKVKV

SEQ ID NO: 16 - protein, 20 aa, Artificial Sequence
  Description of Artificial Sequence Synthetic peptide
  MOD_RES (10)..(10): D-amino acid
VLTKVKTKVPPTKVEVKVLV

SEQ ID NO: 17 - protein, 28 aa, Artificial Sequence
  Description of Artificial Sequence Synthetic peptide
  MOD_RES (10)..(10): D-amino acid
VLTKVKTKVPPTKVEVKVLVGGGGRGDV

SEQ ID NO: 18 - protein, 24 aa, Artificial Sequence
  Description of Artificial Sequence Synthetic peptide
  MOD_RES (14)..(14): D-amino acid
RGDSVKVRVRVRVPPTRVRVRVKV

SEQ ID NO: 19 - protein, 26 aa, Artificial Sequence
  Description of Artificial Sequence Synthetic peptide
  MOD_RES (16)..(16): D-amino acid
IKVAVGVKVRVRVRVPPTRVRVRVKV

SEQ ID NO: 20 - protein, 26 aa, Artificial Sequence
  Description of Artificial Sequence Synthetic peptide
  MOD_RES (16)..(16): D-amino acid
YIGSRGVKVRVRVRVPPTRVRVRVKN

SEQ ID NO: 21 - protein, 18 aa, Artificial Sequence
  Description of Artificial Sequence Synthetic peptide
  MOD_RES (9)..(9): D-amino acid
VLTKVKTVPPTKVEVKVL

SEQ ID NO: 22 - protein, 28 aa, Artificial Sequence
  Description of Artificial Sequence Synthetic peptide
  MOD_RES (10)..(10): D-amino acid
VEVSVSVEVPPTEVSVEVEVGGGGRGDV

SEQ ID NO: 23 - protein, 20 aa, Artificial Sequence
  Description of Artificial Sequence Synthetic peptide
  MOD_RES (10)..(10): D-amino acid
VEVSVSVEVPPTEVSVEVEV